(19) United States
(12) Patent Application Publication
(10) Pub. No.: US 2024/0398564 A1
(43) Pub. Date: Dec. 5, 2024

(54) SYSTEMS AND METHODS FOR FORCE REDUCTION IN DELIVERY SYSTEMS

(71) Applicant: EDWARDS LIFESCIENCES CORPORATION, Irvine, CA (US)

(72) Inventors: Jesse Robert Edwards, Silverado, CA (US); Taylor Jacob Scheinblum, Newport Beach, CA (US); Nikolai Brent Poulsen, Tustin, CA (US); Hieu Minh Luong, Westminster, CA (US); David Robert Landon, Huntington Beach, CA (US); Cooper Ryan Rickerson, Huntington Beach, CA (US); Jessie Ambriz Cornejo, Corona, CA (US); Hiroshi Okabe, Tustin, CA (US)

(21) Appl. No.: 18/798,753

(22) Filed: Aug. 8, 2024

Related U.S. Application Data
(63) Continuation of application No. PCT/US2023/012428, filed on Feb. 6, 2023.
(60) Provisional application No. 63/308,137, filed on Feb. 9, 2022.

(51) Int. Cl. A61F 2/24

(52) U.S. Cl. CPC A61F 2/2436; A61F 2/2418; A61F 2220/0016

(57) ABSTRACT

Examples of the present disclosure may be directed to reducing a tension force or a compression force in one or more shafts of the delivery system. An elongate catheter may include the one or more shafts. The tension force or compression force may result from residual tension or compression in a shaft resulting from movement of the shaft. The tension force or compression force may alternatively or in combination result from interacting forces across a plurality of the shafts. The tension force or compression force may hinder the operation of the delivery system and in instances may damage the delivery system. Systems, devices, and methods for reducing the tension force or compression force are disclosed herein.

### CROSS-REFERENCE TO RELATED APPLICATIONS

This application is a continuation of International Application No. PCT/US2023/012428, filed Feb. 6, 2023, which designates the United States and was published in English by the International Bureau on Aug. 17, 2023 as WO2023/154250, which claims the benefit of U.S. Provisional Application No. 63/308,137, filed Feb. 9, 2022, the entire contents of each of which are hereby incorporated by reference.

### BACKGROUND

### Field

Certain examples disclosed herein relate generally to prostheses for implantation within a lumen or body cavity and delivery systems for a prosthesis. In particular, the prostheses and delivery systems relate in some examples to replacement heart valves, such as replacement mitral heart valves or replacement tricuspid heart valves.

### Description of the Related Art

Human heart valves, which include the aortic, pulmonary, mitral and tricuspid valves, function essentially as one-way valves operating in synchronization with the pumping heart. The valves allow blood to flow downstream, but block blood from flowing upstream. Diseased heart valves exhibit impairments such as narrowing of the valve or regurgitation, which inhibit the valves' ability to control blood flow. Such impairments reduce the heart's blood-pumping efficiency and can be a debilitating and life-threatening condition. For example, valve insufficiency can lead to conditions such as heart hypertrophy and dilation of the ventricle. Thus, extensive efforts have been made to develop methods and apparatuses to repair or replace impaired heart valves.

Prostheses exist to correct problems associated with impaired heart valves. For example, mechanical and tissue-based heart valve prostheses can be used to replace impaired native heart valves. More recently, substantial effort has been dedicated to developing replacement heart valves, particularly tissue-based replacement heart valves that can be delivered with less trauma to the patient than through open heart surgery. Replacement valves are being designed to be delivered through minimally invasive procedures and even percutaneous procedures. Such replacement valves often include a tissue-based valve body that is connected to an expandable frame that is then delivered to the native valve's annulus.

Development of prostheses including but not limited to replacement heart valves that can be compacted for delivery and then controllably expanded for controlled placement has proven to be particularly challenging. An additional challenge relates to the ability of such prostheses to be secured relative to intralumenal tissue, e.g., tissue within any body lumen or cavity, in an atraumatic manner.

Delivering a prosthesis to a desired location in the human body, for example delivering a replacement heart valve to the mitral valve, can also be challenging. Obtaining access to perform procedures in the heart or in other anatomical locations may require delivery of devices percutaneously through tortuous vasculature or through open or semi-open surgical procedures. The ability to control the deployment of the prosthesis at the desired location can also be challenging.

### SUMMARY

Examples of the present disclosure are directed to a delivery system, such as but not limited to a delivery system for an implant. The implant may comprise a prosthesis such as but not limited to a replacement heart valve. Further examples are directed to methods of use to deliver and/or controllably deploy an implant, such as but not limited to a replacement heart valve, to a desired location within the body. In some examples, a replacement heart valve and methods for delivering a replacement heart valve to a native heart valve, such as a mitral valve, an aortic valve, or a tricuspid valve, are provided.

Examples of the present disclosure may be directed to reducing a tension force or a compression force in one or more shafts of the delivery system. An elongate catheter may include the one or more shafts. The tension force or compression force may result from residual tension or compression in a shaft resulting from movement of the shaft. The tension force or compression force may alternatively or in combination result from interacting forces across a plurality of the shafts. The tension force or compression force may hinder the operation of the delivery system and in instances may damage the delivery system. Reduction of the tension force or compression force may be desired.

The reduction of the tension force or the compression force may be produced automatically. A force reduction mechanism may be utilized to automatically reduce a tension force or a compression force in at least one of the one or more shafts. The force reduction mechanism may be utilized to allow the shaft to slide proximally or distally to reduce the tension force or the compression force in the shaft.

In examples, the force reduction mechanism may automatically produce a “back drive” or “back off” of the shaft. For example, a proximal movement of the shaft may produce a tension force in the shaft. The force reduction mechanism may allow the shaft to automatically move distally to reduce the tension force present in the shaft from the proximal movement. Similarly, a distal movement of the shaft may produce a compression force in the shaft. The force reduction mechanism may allow the shaft to automatically move proximally to reduce the compression force present in the shaft. Various examples of force reduction and of force reduction mechanisms are disclosed herein.

In examples, dynamic adapter assemblies may be provided that may be configured to reduce the force within a shaft. Indicators may be provided that may be configured to indicate a high force upon a shaft or an overloaded adapter. Force reduction mechanisms may be disclosed that may be mechanical devices. The mechanical devices may be unpowered. Force reduction mechanisms may be electric in examples and may include electric drives.

The force reduction mechanisms may comprise fail safe mechanisms that may reduce the possibility of damage or failure of a shaft of a delivery system.

In examples, a delivery system for an implant is provided. The delivery system may include an elongate catheter including an implant retention area for retaining the implant, the elongate catheter including one or more shafts. The delivery system may include a control mechanism for moving the one or more shafts. The delivery system may include a force reduction mechanism for automatically reducing a tension force or a compression force in at least one of the one or more shafts upon a respective threshold tension force or threshold compression force being met in the at least one of the one or more shafts.

In examples, a delivery system for an implant is provided. The delivery system may include an elongate catheter including an implant retention area for retaining an implant, the elongate catheter including one or more shafts. The delivery system may include a control mechanism for moving the one or more shafts. The delivery system may include an indicator indicating a tension force or a compression force in at least one of the one or more shafts.

In examples, a method is provided. The method may include utilizing a delivery system to deploy an implant within a patient's body. The delivery system may include an elongate catheter including an implant retention area retaining the implant, the elongate catheter including one or more shafts, a control mechanism for moving the one or more shafts, and a force reduction mechanism configured to automatically reduce a tension force or a compression force in at least one of the one or more shafts.

In examples, a method is provided. The method may include utilizing a delivery system to deploy an implant within a patient's body. The delivery system may include an elongate catheter including an implant retention area for retaining the implant, the elongate catheter including one or more shafts, a control mechanism for moving the one or more shafts, and an indicator indicating a tension force or a compression force in at least one of the one or more shafts.

In examples, a delivery system for an implant is provided. The delivery system may include an elongate catheter including an implant retention area for retaining the implant, the elongate catheter including one or more shafts. The delivery system may include a control mechanism for moving the one or more shafts, the control mechanism including an actuator knob having a first portion and a second portion for rotating relative to the first portion to automatically limit a tension force or a compression force in at least one of the one or more shafts transmitted by the actuator knob to the at least one of the one or more shafts.

In examples, a method is provided. The method may include utilizing a delivery system to deploy an implant within a patient's body. The delivery system may include an elongate catheter including an implant retention area for retaining the implant, the elongate catheter including one or more shafts, and a control mechanism for moving the one or more shafts, the control mechanism including an actuator knob having a first portion and a second portion that is configured to rotate relative to the first portion to automatically limit a tension force or a compression force in at least one of the one or more shafts transmitted by the actuator knob to the at least one of the one or more shafts.

In examples, a delivery system for an implant is provided. The delivery system may include an elongate catheter including an implant retention area for retaining the implant, the elongate catheter including at least one shaft. The delivery system may include an actuator knob on a handle for advancing or retracting the at least one shaft, the actuator knob engaged with the at least one shaft. The delivery system may include a force reduction mechanism for allowing the at least one shaft to disengage from the actuator knob for reducing a tension force in the at least one shaft. The force reduction mechanism may cause the at least one shaft to disengage from the actuator knob upon reaching a threshold tension force for allowing the at least one shaft to slide longitudinally relative to the actuator knob. The force reduction mechanism may reduce damage to the at least one shaft.

### BRIEF DESCRIPTION OF THE DRAWINGS

FIG. 1 shows an example of a delivery system for an implant (such as a dual-frame heart valve prosthesis).

FIG. 2A shows a perspective view of a frame of a dual-frame valve prosthesis that may be delivered using a delivery system described herein.

FIG. 2B shows a perspective view of an inner frame of the dual-frame valve prosthesis of FIG. 2A.

FIG. 2C shows a perspective view of an outer frame of the dual-frame valve prosthesis of FIG. 2A.

FIG. 2D shows a perspective view of a fully-assembled dual-frame valve prosthesis including a skirt assembly and padding.

FIG. 3A shows a perspective view of an example of an outer sheath subassembly of a delivery device of the delivery system of FIG. 1.

FIG. 3B illustrates a side-cross-sectional view of a capsule subassembly of the outer sheath subassembly of FIG. 3A.

FIG. 3C shows a perspective view of a capsule stent, or distal hypotube, of the outer sheath subassembly of FIG. 3A.

FIG. 3D schematically illustrates how a portion of a liner extending along a length of the outer sheath subassembly can have built-in slack to facilitate flexible bending of the outer sheath subassembly.

FIG. 4A shows a perspective view of a rail subassembly of the delivery device of the delivery system of FIG. 1.

FIG. 4B shows a side cross-sectional view of the rail subassembly of FIG. 4A.

FIG. 4C schematically illustrates how an outer compression coil and pull wire can have a longer length than an inner compression coil and pull wire of the rail subassembly.

FIG. 5A shows a perspective view of a mid-shaft subassembly of the delivery device of the delivery system of FIG. 1.

FIG. 5B illustrates a side cross-sectional view of the mid-shaft subassembly of FIG. 5A.

FIG. 6A shows a perspective view of a release subassembly of the delivery device of the delivery system of FIG. 1.

FIG. 6B shows a side cross-sectional view of the release subassembly of FIG. 6A.

FIG. 6C shows a close-up side view of a distal end portion of the release subassembly.

FIG. 6D shows a side cross-sectional view of the distal end portion of the release subassembly.

FIG. 6E shows a bottom view of the distal end of the release subassembly.

FIG. 7A shows a perspective view of a manifold subassembly of the delivery device of the delivery system of FIG. 1.

FIG. 7B shows a side cross-sectional view of the manifold subassembly of FIG. 7A.

FIG. 7C shows a close-up view of a distal end portion of the manifold subassembly.

FIG. 7D shows a bottom view of the distal end portion of the manifold subassembly.

FIG. 7E shows a flat cut pattern of a distal end portion of the manifold subassembly.

FIGS. 8A and 8B show distal end portions of the release and manifold subassemblies in a locked configuration and unlocked configuration, respectively.

FIG. 8C illustrates tethering and untethering of a suture using the release and manifold subassemblies.

FIG. 8D shows suture loops tethered to the eyelets of a valve prosthesis while also tethered to the manifold subassembly of the delivery device.

FIG. 9A shows a perspective view of a handle of the delivery device of FIG. 1.

FIG. 9B shows a side cross-sectional view of the handle of the delivery device.

FIG. 10A shows how the handle of the delivery device interfaces with an example of a stabilizer assembly of the delivery system of FIG. 1.

FIG. 10B shows a perspective view of the stabilizer assembly without the delivery device attached.

FIG. 10C shows a top view of the stabilizer assembly of FIG. 10A.

FIG. 11 shows a schematic representation of a transfemoral and transseptal delivery approach.

FIG. 12 shows a schematic representation of a valve prosthesis positioned within a native mitral valve (shown without a skirt assembly to facilitate visualization of interface with native heart valve structures).

FIGS. 13A-13F show various steps of deployment and recapture of the valve prosthesis using the delivery device described herein.

FIG. 14 shows a perspective view of a handle of a delivery device.

FIG. 15 shows a cross sectional view of the handle of the delivery device shown in FIG. 14 along line A-A.

FIG. 16 shows a top view of the handle of the delivery device shown in FIG. 14.

FIG. 17 shows a perspective view of a distal portion of a handle of a delivery device.

FIG. 18 shows a perspective cross sectional view of the distal portion of the handle of the delivery device shown in FIG. 17 along line B-B.

FIG. 19 shows a side cross sectional view of the distal portion of the handle of the delivery device shown in FIG. 17 along line B-B.

FIG. 20 shows a perspective view of a handle of a delivery device.

FIG. 21 shows a cross sectional view of the handle of the delivery device shown in FIG. 20 along line C-C.

FIG. 22 shows a cross sectional view of a handle of a delivery device.

FIG. 23 shows a cross sectional view of the handle of the delivery device shown in FIG. 22.

FIG. 24 shows a perspective view of a handle of a delivery device.

FIG. 25 shows a cross sectional view of a handle of a delivery device.

FIG. 26 shows a cross sectional view of the handle of the delivery device shown in FIG. 25.

FIG. 27 shows a cross sectional view of a handle of a delivery device.

FIG. 28 shows a cross sectional view of the handle of the delivery device shown in FIG. 27.

FIG. 29 shows a cross sectional view of a handle of a delivery device.

FIG. 30 shows a cross sectional view of the handle of the delivery device shown in FIG. 29.

FIG. 31 shows a cross sectional view of a handle of a delivery device.

FIG. 32 shows a cross sectional view of the handle of the delivery device shown in FIG. 31.

FIG. 33 shows a cross sectional view of a handle of a delivery device.

FIG. 34 shows a cross sectional view of a handle of a delivery device.

FIG. 35 shows a side view of a handle of a delivery device.

FIG. 36 shows a perspective view of an adapter.

FIG. 37 shows a cross sectional view of a handle of a delivery device.

FIG. 38 shows a perspective cross sectional view of a handle of a delivery device.

FIG. 39 shows a perspective cross sectional view of a portion of a handle of a delivery device.

FIG. 40 shows an end cross sectional view of the portion of the handle of the delivery device shown in FIG. 39.

FIG. 41 shows a perspective cross sectional view of a portion of a handle of a delivery device.

FIG. 42 shows an end cross sectional view of the portion of the handle of the delivery device shown in FIG. 41.

FIG. 43 illustrates a perspective view of an actuator knob.

FIG. 44 illustrates a side view of an inner portion of the actuator knob shown in FIG. 43.

FIG. 45 illustrates a side cross sectional view of an outer portion of the actuator knob shown in FIG. 43.

FIG. 46 illustrates a side cross sectional view of the actuator knob shown in FIG. 43.

FIG. 47 illustrates a side cross sectional view of the actuator knob shown in FIG. 43 positioned upon a handle.

FIG. 48 illustrates a perspective view of an actuator knob.

FIG. 49 illustrates a perspective view of an inner portion of the actuator knob shown in FIG. 48.

FIG. 50 illustrates a perspective view of an outer portion of the actuator knob shown in FIG. 48.

FIG. 51 illustrates a side cross sectional view of the actuator knob shown in FIG. 48.

FIG. 52 illustrates a cross sectional view of the actuator knob shown in FIG. 48 at a view perpendicular to the view shown in FIG. 51.

FIG. 53 illustrates a perspective view of an actuator knob.

FIG. 54 illustrates a side cross sectional view of the actuator knob shown in FIG. 53.

FIG. 55 illustrates a perspective view of the actuator knob shown in FIG. 53 with an outer portion shown in transparency.

FIG. 56 illustrates a cross sectional view of the actuator knob shown in FIG. 53.

FIG. 57 illustrates a perspective view of an actuator knob.

FIG. 58 illustrates a side cross sectional view of the actuator knob shown in FIG. 57.

FIG. 59 illustrates a perspective view of the actuator knob shown in FIG. 57 with portions shown in transparency.

### DETAILED DESCRIPTION

The present specification and drawings provide aspects and features of the disclosure in the context of several examples of implants such as replacement heart valves, and delivery systems and methods that are configured for use in the vasculature of a patient, such as for replacement of natural heart valves in a patient. These examples may be discussed in connection with replacing specific valves such as the patient's aortic, tricuspid, or mitral valve. However, it is to be understood that the features and concepts discussed herein can be applied to products other than heart valve implants. For example, the controlled positioning, deployment, and securing features described herein can be applied to other medical implants, for example other types of expandable prostheses, for use elsewhere in the body, such as within an artery, a vein, or other body cavities or locations. In addition, particular features of a valve, delivery system, etc. should not be taken as limiting, and features of any one example discussed herein can be combined with features of other examples as desired and when appropriate. While certain of the examples described herein are described in connection with a transfemoral delivery approach, it should be understood that these examples can be used for other delivery approaches such as, for example, transapical or transjugular approaches. Moreover, it should be understood that certain of the features described in connection with some examples can be incorporated with other examples, including those which are described in connection with different delivery approaches.

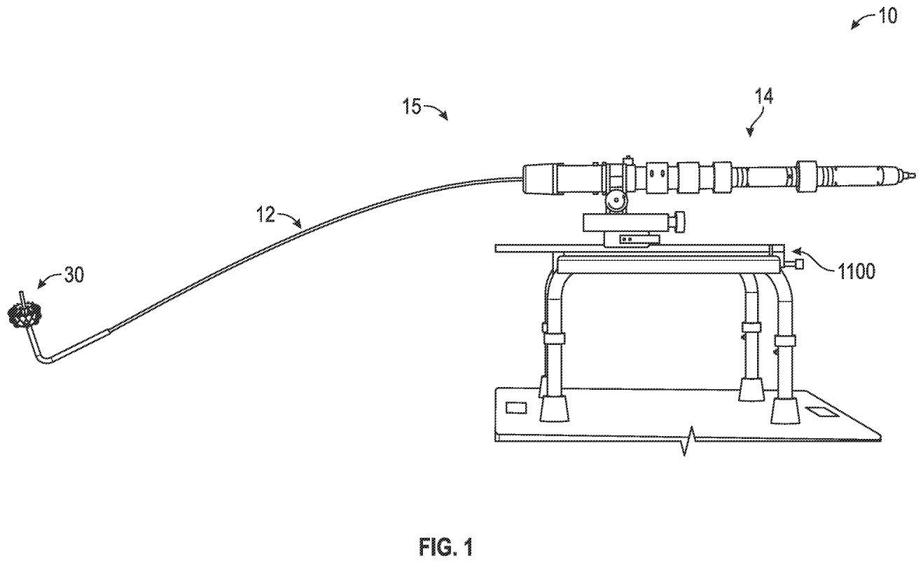

FIG. 1 illustrates an example of a delivery system 10. The delivery system 10 can be used to deploy an implant such as a prosthesis. The prosthesis, for example, may comprise a replacement heart valve to be deployed to a location within a body of a subject (e.g., human or veterinary subject). Replacement heart valves can be delivered to a subject's heart mitral or tricuspid valve annulus or other heart valve location in various manners, such as by open surgery, minimally-invasive surgery, and percutaneous or transcatheter delivery through the subject's vasculature. Example transfemoral approaches are described further in U.S. Pat. Publ. No. 2015/0238315, published Aug. 27, 2015, the entirety of which is hereby incorporated by reference in its entirety. While the delivery system 10 is described in connection with a percutaneous delivery approach, and more specifically a transfemoral delivery approach, it should be understood that features of delivery system 10 can be applied to other delivery approaches, including delivery systems for a transapical delivery approach.

The delivery system 10 can be used to deploy a prosthesis, such as a replacement heart valve as described elsewhere in this specification, to a location within the body of a subject. The delivery system 10 can include multiple components, devices, or subassemblies. As shown in FIG. 1, the delivery system 10 can include an elongate catheter or delivery device 15, and a stabilizer assembly 1100, and other components as desired. The delivery device 15 may include an elongate shaft or shaft assembly 12 and a housing in the form of a handle 14. The shaft assembly 12 may include one or more shafts. A plurality of shafts may be provided according to examples herein, although in examples a single shaft may be utilized.

The elongate catheter or delivery device 15 can be pre-attached to an implant (e.g., a valve prosthesis or replacement heart valve) 30 and the delivery device 15 may be configured to facilitate delivery and implantation of the implant 30 to and at a desired target location (e.g., a mitral or tricuspid heart valve annulus, among other locations). The implant 30 may be pre-attached within a distal end portion of the shaft assembly 12 and removably tethered to one or more retention components of the shaft assembly 12 during manufacturing or assembly. The pre-loaded delivery device 15 may then be packaged, sterilized, and shipped for use by one or more clinicians. In accordance with several examples, the delivery device 15 is ready for use upon removal from its packaging and may not require loading of the implant by a clinician. In examples, the delivery device 15 may be flushed and loaded prior to use.

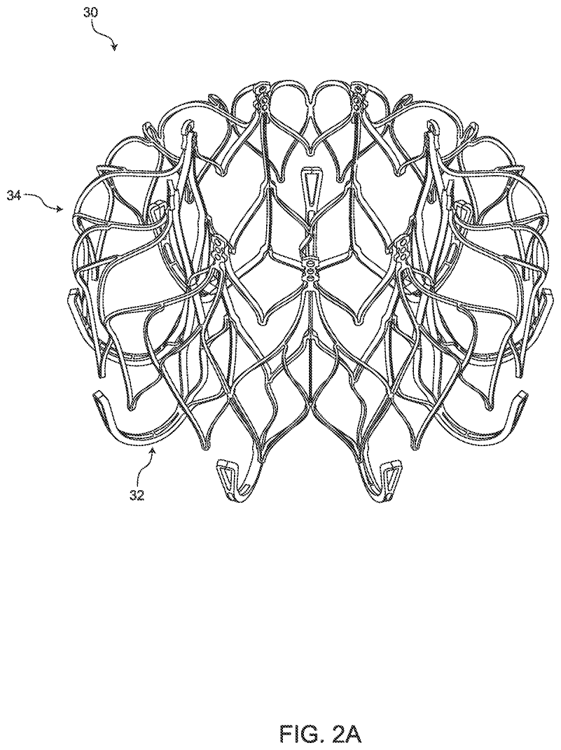

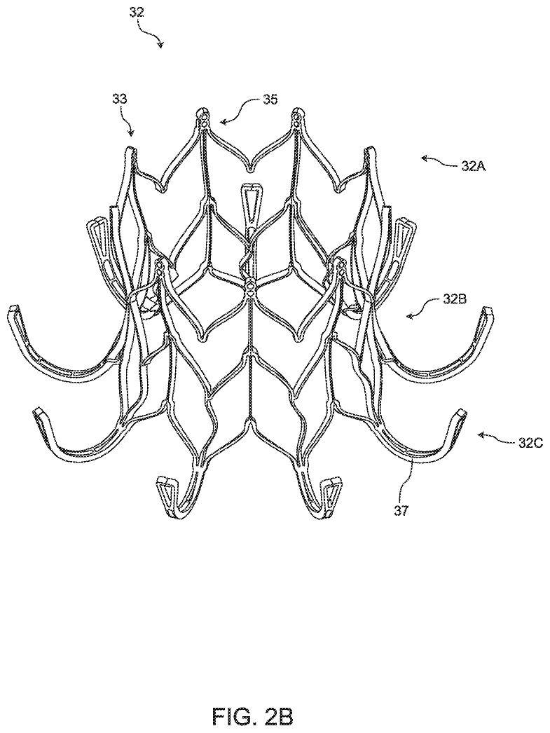

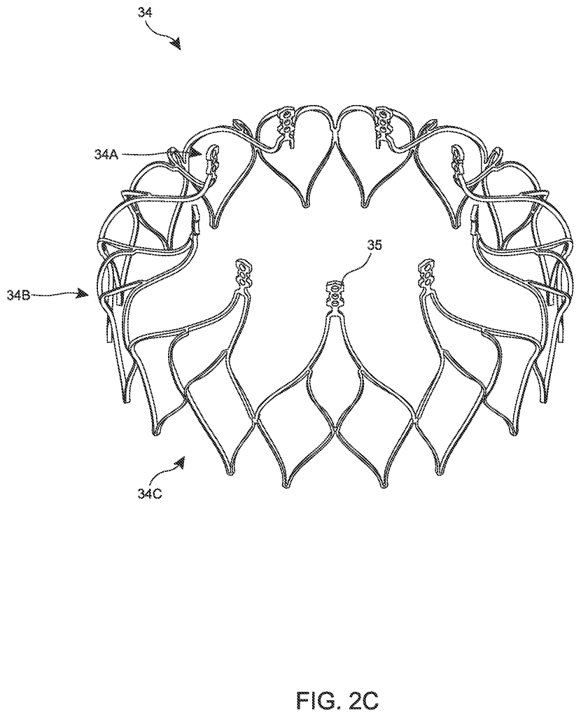

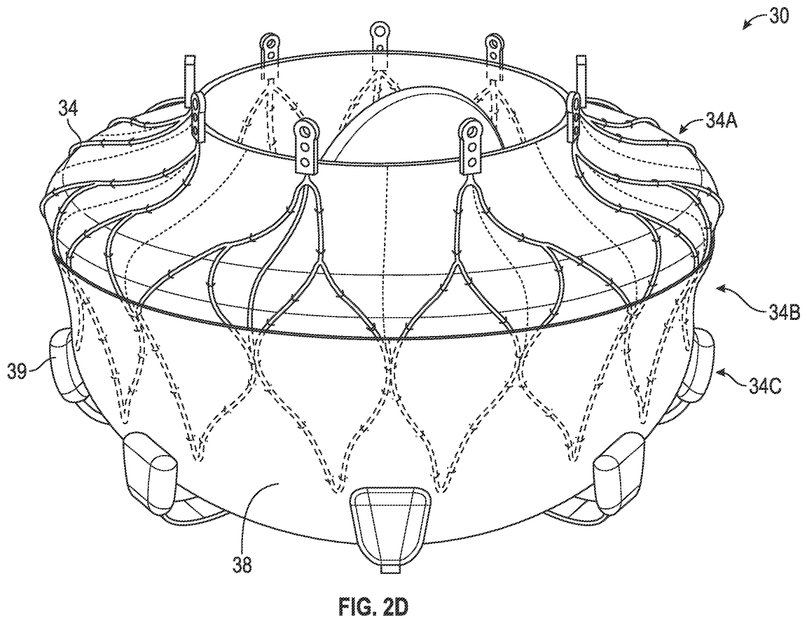

FIGS. 2A-2D show an example of an implant (e.g., a valve prosthesis or replacement heart valve) 30 that can be pre-loaded into and delivered by the delivery device 15. The implant 30 may include a dual frame assembly including an inner frame 32 and an outer frame 34 that are aligned and coupled together during manufacture.

FIG. 2B illustrates an example of the inner frame 32. The inner frame 32 can include a proximal, or inflow, portion 32A, a middle, or intermediate, portion 32B, and a distal, or outflow, portion 32C. The inner frame 32 can be shaped to exhibit a generally hourglass shape in an expanded configuration, in which the middle portion 32B has a smaller cross-sectional width than the cross-sectional width of the proximal portion 32A and the distal portion 32C. The proximal portion 32A may include tabs 33 and/or eyelets 35 to facilitate engagement with other structures or materials (e.g., the outer frame 34, a skirt or fabric assembly, a prosthetic valve assembly, and/or tethers or retention sutures of the elongate catheter or delivery device 15). The distal portion 32C may include outwardly and upwardly-extending anchors 37 to facilitate anchoring at a desired target location (e.g., a native heart annulus). The inner frame 32 may have a chevron cell structure as shown in FIG. 2B. However, other cell structures may be used. The inner frame 32 may include a prosthetic valve assembly coupled thereto comprising a plurality of prosthetic valve leaflets (not shown).

FIG. 2C illustrates an example of the outer frame 34. The outer frame 34 may also include a proximal, or inflow, portion 34A, a middle, or intermediate, portion 34B, and a distal, or outlet, portion 34C. Similar to the proximal portion 32A of the inner frame 32, the proximal portion 34A of the outer frame 34 may also include one or more eyelets 35 to facilitate coupling to one or more structures or materials (e.g., the inner frame 32, a skirt or fabric assembly, and/or to tethers or retention sutures of the elongate catheter or delivery device 15). For case of understanding, in FIGS. 2A-2C, the implant 30 is shown with only the bare metal frame structures illustrated.

FIG. 2D illustrates an example of a fully-assembled implant (e.g., valve prosthesis or replacement heart valve) 30 including a skirt assembly 38 coupled to the frames 32, 34 and including padding 39 surrounding the anchors 37. The implant 30 can take any number of different forms or designs.

Additional details and example designs for an implant (e.g., prosthesis or replacement heart valve) are described in U.S. Pat. Nos. 8,403,983, 8,414,644, 8,652,203 and U.S. Patent Publication Nos. 2011/0313515, 2012/0215303, 2014/0277390, 2014/0277422, 2014/0277427, 2018/0021129, 2018/0055629 and 2019/0262129 (e.g., hourglass shape of inner frame). The entirety of these patents and publications are hereby incorporated by reference and made a part of this specification. Further details and examples of a replacement heart valve or prosthesis and its method of implantation are described in U.S. Publication Nos. 2015/0328000, 2016/0317301, 2019/0008640, and 2019/0262129, the entirety of each of which is hereby incorporated by reference and made a part of this specification.

Referring briefly back to FIG. 1, the elongate catheter or delivery device 15 can include an elongate shaft or shaft assembly 12 comprising a proximal end and a distal end, with a handle 14 coupled to the proximal end of the shaft assembly 12. The elongate catheter or delivery device 15 can be used to hold the implant (e.g., prosthesis, replacement heart valve) for advancement of the same through the vasculature to a treatment location. In some examples, the elongate shaft or shaft assembly 12 can hold at least a portion of an expandable implant (e.g., prosthesis, replacement heart valve) in a compressed state for advancement of the implant within the body. The elongate shaft or shaft assembly 12 may then be used to allow controlled expansion of the implant at a desired implantation location (e.g. treatment location). In some examples, the shaft assembly 12 may be used to allow for sequential controlled expansion of the implant as discussed in detail below.

The elongate shaft or shaft assembly 12 of the delivery device 15 can include one or more shafts. In examples, a plurality of shafts may be provided. The plurality of shafts may include one or more subassemblies or shafts, such as an outer sheath shaft or subassembly 20, a rail shaft or subassembly 21, a mid shaft or mid shaft subassembly 22, a release shaft or release subassembly 23, a manifold shaft or subassembly 24, and/or a nose cone shaft or subassembly, as will be described in more detail below. In some examples, the shaft assembly 12 of the elongate catheter or delivery device 15 may not have all of the subassemblies or shafts disclosed herein. The delivery device 15 may include multiple layers of concentric shafts, subassemblies, or lumens. The various lumen or shaft subassemblies will be described starting from an outermost layer. In some examples, the shafts or subassemblies disclosed below may be in a different radial order than is discussed.

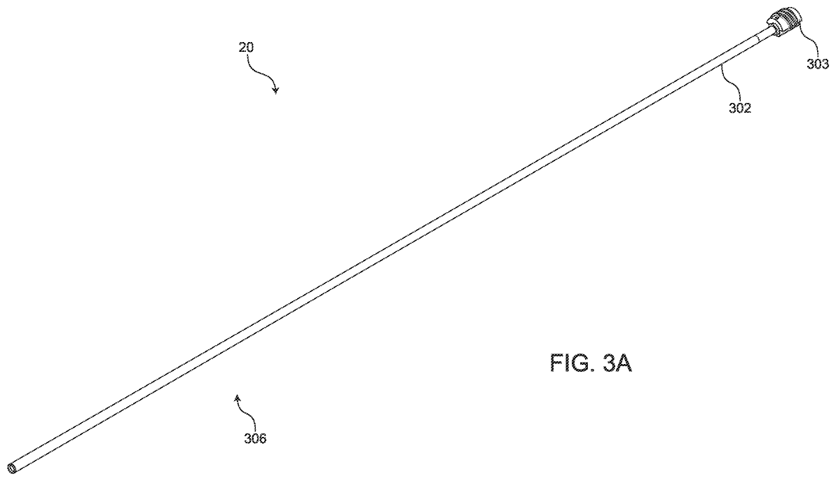

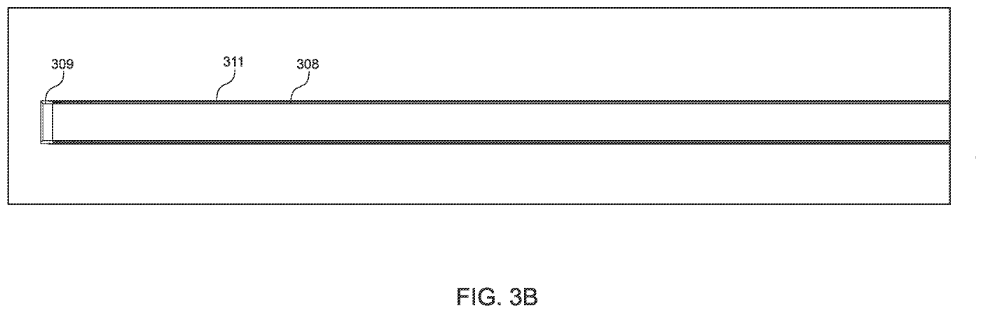

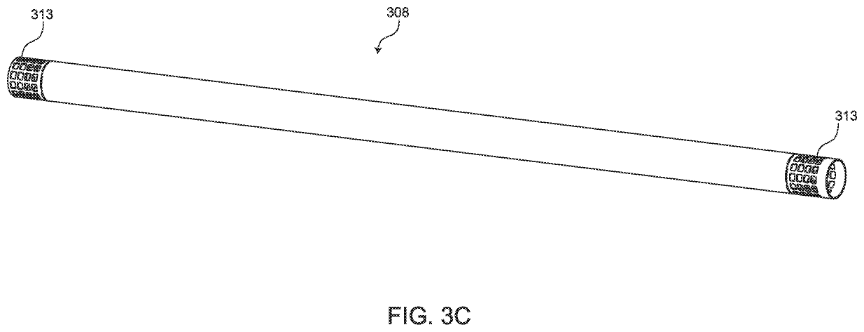

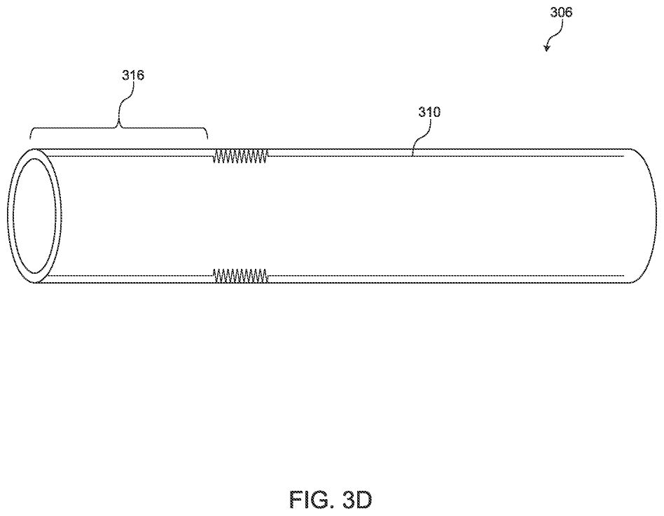

FIG. 3A shows a perspective view of an example of the outer sheath shaft or subassembly 20 of the elongate catheter or delivery device 15 of the delivery system 10. The outer sheath shaft or subassembly 20 forms a radially outer covering, or sheath, to cover and surround an implant retention area for retaining the implant, and prevent at least a portion of the implant (e.g., replacement heart valve or valve prosthesis) 30 from radially expanding until ready for implantation. Specifically, the outer sheath subassembly 20 can prevent a distal end portion of the implant 30 from radially expanding.

The outer sheath shaft or subassembly 20 can include an outer proximal shaft 302 having a proximal end portion operably coupled (e.g., via threaded outer sheath adapter 303 at a proximal portion of the outer sheath shaft or subassembly 20) to a capsule actuator or knob 905 (which may be a distal-most actuator or knob, as shown in FIGS. 9A and 9B) of the handle 14 such that rotation of the capsule knob 905 causes proximal and distal movement in the form of translation of the outer sheath subassembly 20 (e.g., clockwise and counter-clockwise rotation). A capsule subassembly 306 can be attached to a distal end of the outer proximal shaft 302. The components of the outer sheath shaft or subassembly 20 can form an outer-most lumen for the other shafts or subassemblies to pass through.

The outer proximal shaft 302 may be a tube formed of a plastic, but could also be formed of a metal hypotube or other material. The outer proximal shaft 302 may include an outer jacket or liner made of fluorinated ethylene propylene (FEP) material, polytetrafluoroethylene (PTFE) material, ePTFE material, or other polymeric material so as to make the outer surface of the outer proximal shaft 302 smooth and hemostatic. The outer proximal shaft 302 may include a connector (e.g., flexible reflow member) at its distal end to facilitate connection or coupling to the capsule subassembly 306. At least a portion of the outer proximal shaft 302 may comprise a laser cut hypotube with a flexible pattern, such as a universally flexible pattern. An interrupted spiral pattern or an interrupted coil may be utilized.

FIG. 3B shows a side cross-section view of the capsule subassembly 306. The capsule subassembly 306 may include a distal hypotube, or capsule stent, 308, an inner liner inside of the hypotube 308, a distal capsule tip 309, and one or more outer liners or jackets 311 surrounding the hypotube 308. The one or more outer liners or jackets 311 may comprise PEBAX or other suitable polymer or thermoplastic elastomer material, such as polytetrafluoroethylene (PTFE) or expanded polytetrafluoroethylene (ePTFE). The inner liner may comprise PTFE, which may be pre-compressed before application to the inside of the hypotube 308. The distal capsule tip 309 may comprise an atraumatic tip adapted to act as a funnel to facilitate recapture (e.g., crimping) of a valve prosthesis or other implant. The distal capsule tip 309 may be comprised of polyetheretherketone (PEEK) or other thermoplastic, polymeric, or metallic material. The distal capsule tip 309 may be loaded with radiopaque material (e.g., 5-40% barium sulfate loading) to facilitate detection (e.g., made fluorogenic) under radiographic imaging (e.g., fluoroscopy). The distal capsule tip 309 may fit within an open distal end of the hypotube 308.

FIG. 3C shows a perspective view of the distal hypotube, or capsule stent 308. The capsule stent 308 can be formed from one or more materials, such as PTFE, ePTFE, polyether block amide (Pebax®), polyetherimide (Ultem®), PEEK, urethane, Nitinol, stainless steel, and/or any other biocompatible material. The capsule stent 308 is preferably flexible while still maintaining a sufficient degree of radial strength to maintain an implant (e.g., replacement valve) 30 within the capsule stent 308 without substantial radial deformation, which could increase friction between the capsule stent 308 and an implant contained therein. The capsule stent 308 also preferably has sufficient column strength to resist buckling, and sufficient tear resistance to reduce or eliminate the possibility of the implant tearing and/or damaging the capsule stent 308. The proximal end and/or distal end of the distal hypotube, or capsule stent 308 may include multiple laser cut windows 313 adapted to make the proximal and/or distal end fluorogenic and/or echogenic to facilitate visualization under certain imaging modalities (e.g., noninvasive ultrasound imaging or invasive fluoroscopic imaging). In several implementations, a separate radiopaque element or member is not added to the hypotube 308 to facilitate imaging because of the presence of the laser cut windows 313. The laser cut windows 313 may also promote adhesion of the outer jacket 311 to the capsule stent 308 and to the inner liner(s) by allowing glue or other adhesive to flow through the laser cut windows 313. One or more layers of connection members made of PEBAX or other suitable material may surround the laser cut windows 313 to facilitate coupling of the hypotube, or capsule stent 308 to the distal capsule tip 309.

The hypotube 308 may be formed of a plastic or metallic material. In some implementations, the hypotube 308 can be a metal hypotube. If metallic, the metallic material of the hypotube 308 may comprise cobalt chrome, stainless steel, titanium or metal alloy, such as nickel-titanium alloy material. The coil construction or cut patterns of the proximal outer shaft 302 and/or the hypotube 308 can allow the proximal shaft 302 to follow the rail shaft or subassembly 21 in any desired direction. A cut pattern of the proximal outer shaft 302 and/or the hypotube 308 may be modified (e.g., cut per revolution, pitch, spine distance) to control tension resistance, compression resistance, flexibility, and torque resistance. For example, cuts per revolution may range between 1.5 and 5.5, pitch may range between 0.005″ and 0.15″, and spine distance may range between 0.015″ and 0.125″. The hypotube 308 may advantageously provide both tension and compression. The one or more outer liners or jackets 311 may allow the capsule subassembly 306 to be more flexible. The capsule hypotube 308 can bend in multiple directions. In some implementations, a distal terminus of the outer liner or jacket 311 may be positioned proximal of the distal terminus of the hypotube 308.

The capsule subassembly 306 may have a similar diameter as the outer proximal shaft 302 or a different diameter. In some examples, the capsule subassembly 306 has a uniform or substantially uniform diameter along its length. In some examples, the capsule subassembly 306 can be 28 French or less in size (e.g., 27 French). In some examples, the capsule subassembly 306 may include a larger diameter distal portion and a smaller diameter proximal portion. The capsule subassembly 306 can be configured to retain the implant (e.g., valve prosthesis) 30 in the compressed position within the capsule subassembly 306 (e.g., within an implant retention area 316 marked in FIG. 3D occupying a distal-most 5 cm or inches of the capsule subassembly 306.) Additional structural and operation details of a capsule subassembly, such as those described in connection with capsules in U.S. Publication No. 2019/0008640 and U.S. Publication No. 2019/0008639, which are hereby incorporated by reference herein, may be incorporated into the capsule subassembly 306.

The outer sheath shaft or subassembly 20 is configured to be individually movable or slidable with respect to the other shafts or assemblies by operation of a control mechanism. The control mechanism may include an actuator in the form of one or more control knobs. The actuators may comprise an actuator knob or capsule knob 905 (marked in FIG. 9A). The capsule knob 905 may be rotated to move or slide the outer sheath shaft or subassembly 20. Further, the outer sheath subassembly 20 can slide distally and proximally relative to the rail subassembly 21 together with the mid shaft subassembly 22, manifold subassembly 24, release subassembly 23, and/or nose cone subassembly.

FIG. 3D schematically illustrates how at least a portion of a length of one or more components of the capsule subassembly 306 (e.g., inner liner 310) can include excess material such that the capsule subassembly 306 includes built-in slack along a portion of its length (e.g., a portion of the length proximal to the implant retention area 316) to facilitate flexible bending of the capsule subassembly 306 (e.g., to navigate tight turns within a heart or vasculature surrounding the heart).

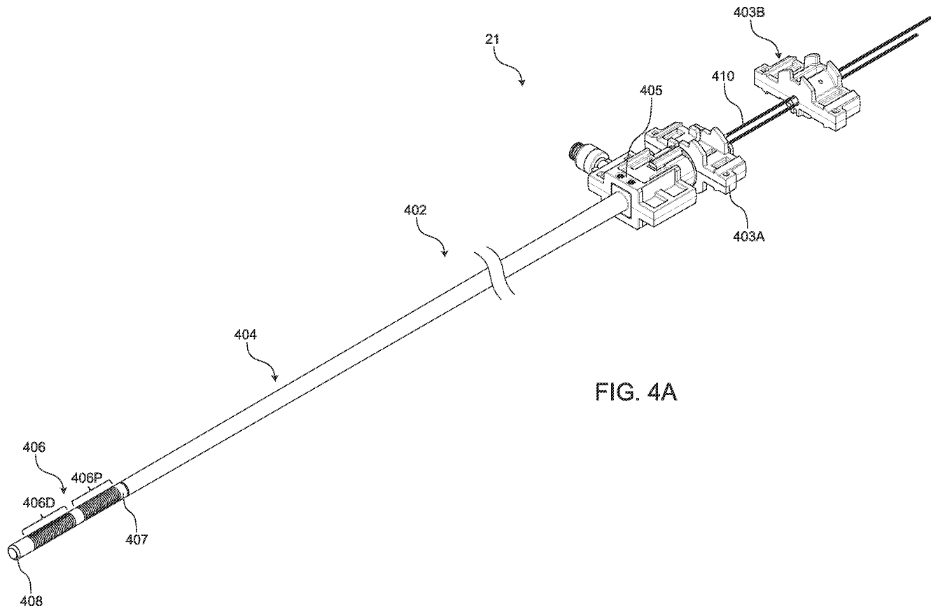

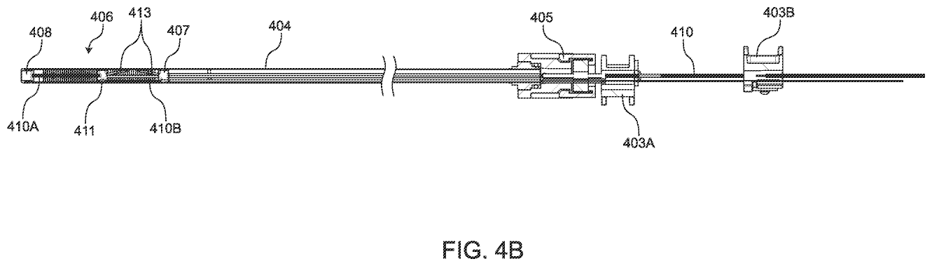

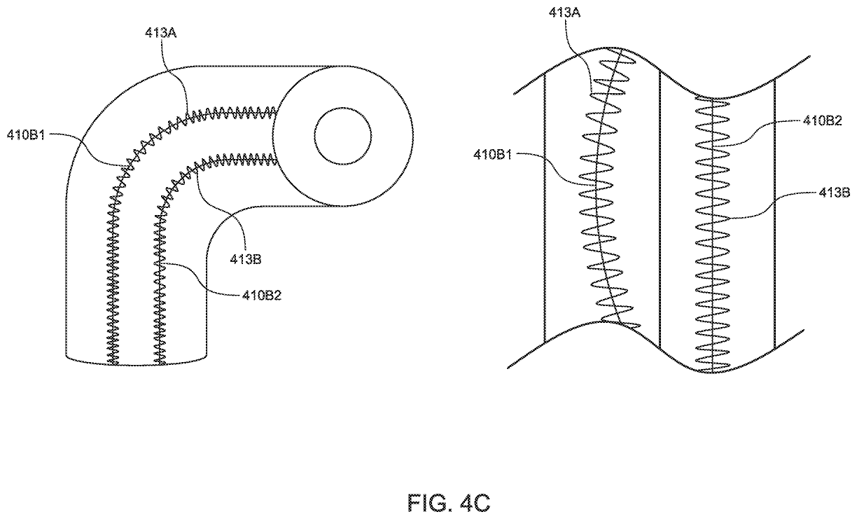

FIG. 4A shows a perspective view of a rail shaft or subassembly 21 of the elongate catheter or delivery device 15 of the delivery system 10 of FIG. 1. FIG. 4A shows approximately the same view as FIG. 3A, but with the outer sheath subassembly 20 removed, thereby exposing the rail subassembly 21.

FIG. 4B further shows a cross-section of the proximal and distal end portions of the rail subassembly 21 to view the pull wires that facilitate steering of the rail subassembly 21. The rail subassembly 21 can include a rail shaft 402 (or rail) generally attached (and operably coupled) at its proximal end to the handle 14. The rail shaft 402 can be made up of a rail proximal shaft 404 directly attached to the handle 14 at a proximal end and a rail hypotube 406 attached to the distal end of the rail proximal shaft 404 (e.g., via a connector, ring-like structure, or insert 407). The rail subassembly 21 is operably coupled to the handle 14 via primary flex adapter 403A at a proximal portion of the rail subassembly 21 (which controls medial-lateral trajectory of the distal end portion of the rail subassembly 21 via one or more distal pull wires 410A (marked in FIG. 4B)), via secondary flex adapter 403B at a proximal portion of the rail subassembly 21 (which controls anterior-posterior trajectory of the distal end portion of the rail subassembly 21 via one or more proximal pull wires 410B), and via rail adapter 405 at a proximal portion of the rail subassembly 21 (which includes a side needleless injection port to facilitate flushing and de-airing functions). The rail proximal shaft 404 may include an interrupted spiral cut pattern along a large portion of its length to facilitate compression. The rail hypotube 406 can further include an atraumatic rail tip 408 at its distal tip. The atraumatic rail tip 408 may not comprise slits and is configured to extend up to 1 inch beyond the distal terminus of the rail hypotube 406 and is configured not to dig into the outer shaft subassembly 20 to avoid friction and fatigue and to prolong use. These components of the rail subassembly 21 can form a lumen for the other inner subassemblies to pass through.

FIG. 4B shows a side cross-section view of the rail shaft or subassembly 21 of FIG. 4A. As shown in FIG. 4B, attached to an inner surface of the rail hypotube 406 are one or more pull wires 410 which can be used apply forces to the rail hypotube 406 and steer the rail subassembly 21. The pull wires 410 can extend distally from the primary and secondary actuator or flex knobs 915A, B (illustrated in FIGS. 9A and 9B) in the handle 14 to the rail hypotube 406. In some examples, pull wires 410 can be attached at different longitudinal locations on the rail hypotube 406, thus providing for multiple bending locations in the rail hypotube 406, allowing for multidimensional steering. For example, the rail hypotube 406 may provide a primary bend or flex along a medial/lateral trajectory and a secondary bend or flex along an anterior/posterior trajectory.

The rail hypotube 406 can include a number of circumferential slots (e.g., laser cut into the hypotube) to facilitate bending and flexibility. The rail hypotube 406 can generally be broken into a number of different sections. At the most proximal end is an uncut (or unslotted) hypotube section corresponding to the location of insert 407. Moving distally, the next section is the proximal slotted hypotube section 406P. This section includes a number of circumferential slots cut into the rail hypotube 406. Generally, two slots are cut around each circumferential location forming almost half of the circumference. Accordingly, two backbones are formed between the slots extending up the length of the rail hypotube 406. This is the section that can be guided by the proximal pull wire(s) 410B. Moving further distally is the location where the proximal pull wires 410 connect, and thus slots can be avoided. This section is just distal of the proximally slotted section 406P and may correspond to the location of insert, or pull wire connector, 411.

Distally following the proximal pull wire connection area is the distal slotted hypotube section 406D. This section is similar to the proximal slotted hypotube section 406P, but may have significantly more slots cut out in an equivalent length. Thus, the distal slotted hypotube section 406D may provide easier bending and an increased bend angle than the proximal slotted hypotube section 406P. In some examples, the proximal slotted section 406P can be configured to experience a bend of approximately 90 degrees with a half inch radius whereas the distal slotted section 406D can bend at approximately 180 degrees with a half inch radius. Further, as shown in FIGS. 4A and 4B, the spines of the distally slotted hypotube section 406D are circumferentially offset from the spines of the proximally slotted hypotube section 406P. Accordingly, the two sections will achieve different bend patterns, allowing for three-dimensional steering of the rail subassembly 21. In some examples, the spines can be offset 30, 45, or 90 degrees, though the particular offset is not limiting. At the distal-most end of the distal slotted hypotube section 406D is the distal pull wire connection area which is again a non-slotted section of the rail hypotube 406.

In some examples, one distal pull wire 410A can extend to a distal section (e.g., to rail tip 408) of the rail hypotube 406 and two proximal pull wires 410B can extend to a proximal section of the rail hypotube 406; however, other numbers of pull wires can be used, and the particular amount of pull wires is not limiting. For example, two distal pull wires 410A can extend to a distal location and a single proximal pull wire 410B can extend to a proximal location. In some examples, ring-like structures or inserts attached inside the rail hypotube 406, known as pull wire connectors, can be used as attachment locations for the proximal pull wires 410B, such as insert 411. In some examples, the pull wires 410 can directly connect to an inner surface of the rail hypotube 406.

The distal pull wire(s) 410A can be connected (either on its own or through rail tip connector 408) generally at the distal end of the rail hypotube 406. The proximal pull wire(s) 410B can connect (either on their own or through the insert 411) at a location approximately one quarter, one third, or one half of the length up the rail hypotube 406 from the proximal end. In some examples, the distal pull wire(s) 410A can pass through a small diameter pull wire lumen (e.g., tube, hypotube, cylinder) attached on the inside of the rail hypotube 406. This can prevent the pull wires 410 from pulling on the rail hypotube 406 at a location proximal to the distal connection. Further, the lumen can comprise compression coils to strengthen the proximal portion of the rail hypotube 406 and prevent unwanted bending. Thus, in some examples the lumen is only located on a proximal portion (e.g., proximal half) of the rail hypotube 406. In some examples, multiple lumens, such as spaced longitudinally apart or adjacent, can be used per distal pull wire 410A. In some examples, a single lumen is used per distal wire 410A. In some examples, the lumen can extend into the distal portion (e.g., distal half) of the rail hypotube 406. In some examples, the lumen is attached on an outer surface of the rail hypotube 406. In some examples, the lumen is not used. In some examples, one or more compression coils 413 extend from the insert 407 to the insert 411. The compression coils 413 may be configured to bypass load in length between a distal primary flex point and a proximal secondary flex point. The compression coils 413 facilitate independent flex planes so that both planes of flex do not activate when one plane of flex is desired to flex. The compression coils 413 may allow for the proximally slotted hypotube section 406P to retain rigidity for specific bending of the distally slotted hypotube section 406D. The compression coils 413 may isolate force so only the primary flex is flexed.

For the pair of proximal pull wires 410B, the wires can be spaced approximately 180° from one another to allow for steering in both directions. Similarly, if a pair of distal pull wires 410A is used, the wires can be spaced approximately 180° from one another to allow for steering in both directions. In some examples, the pair of distal pull wires 410A and the pair of proximal pull wires 410B can be spaced approximately 90° from each other. Opposing wires could be used to provide anti-flex mechanism. In some examples, the pair of distal pull wires 410A and the pair of proximal pull wires 410B can be spaced approximately 0° from each other. However, other locations for the pull wires can be used as well, and the particular location of the pull wires is not limiting. In some examples, the distal pull wire 410A can pass through a lumen attached within the lumen of the rail hypotube 406. This can prevent an axial force on the distal pull wire 410A from creating a bend in a proximal section of the rail hypotube 406. The rail subassembly 21 is disposed so as to be slidable over the radially inner subassemblies. As the rail hypotube 406 is bent, it presses against the other subassemblies to bend them as well, and thus the other subassemblies of the delivery device 15 can be configured to steer along with the rail subassembly 21 as a cooperating single unit, thus providing for full steerability of the distal end of the delivery device 15. Additional structural and operation details of a rail subassembly, such as those described in connection with rail assemblies in U.S. Publication No. 2019/0008640 and U.S. Publication No. 2019/0008639, which are hereby incorporated by reference herein, may be incorporated into the rail subassembly 21.

FIG. 4C schematically illustrates how an outer compression coil 413A and proximal pull wire 410B1 can have a longer length than an inner compression coil 413B and proximal pull wire 410B2 of the rail subassembly 21 so that they don't occupy the same space and to facilitate case of bending in one direction and reduce lumen obstruction during bending.

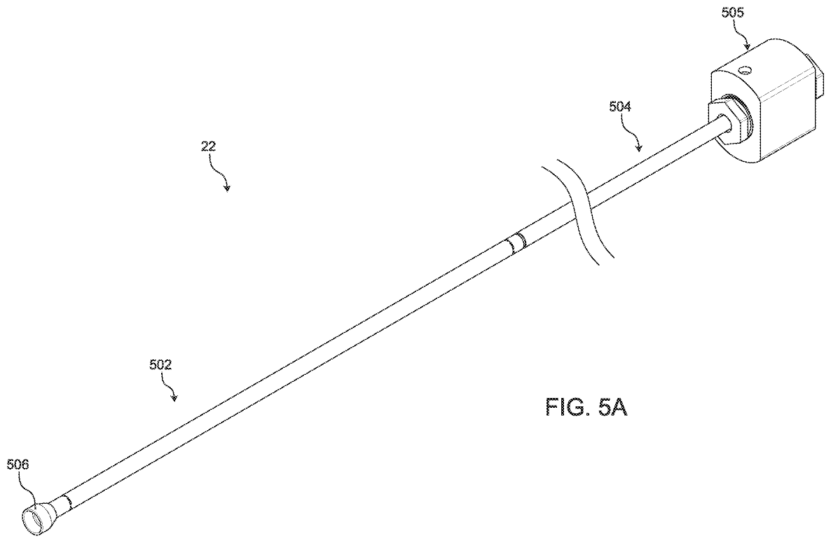

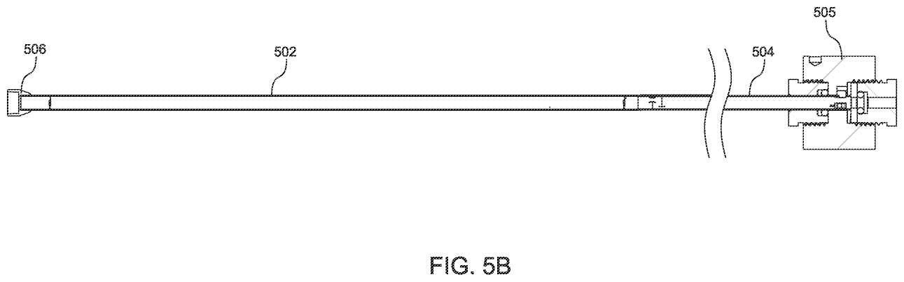

Moving radially inwardly, the next subassembly is the mid shaft or mid shaft subassembly 22. FIG. 5A shows a perspective view of the mid-shaft subassembly 22 of the delivery device 15 of the delivery system 10. The mid-shaft subassembly 22 can include a distal mid-shaft hypotube 502 generally attached at its proximal end to a proximal shaft 504, which in turn can be attached at its proximal end to the handle 14 (e.g., via mid-shaft adapter 505 at a proximal portion of the mid-shaft subassembly 22), and a distal pusher 506 located at the distal end of the mid-shaft hypotube 502. These components of the mid-shaft subassembly 22 can form a lumen for other inner subassemblies to pass through.

The mid-shaft subassembly 22 can be located within a lumen of the rail subassembly 21. The mid-shaft hypotube 502 can be formed of metallic alloy (e.g., cobalt chrome, nickel-chromium-cobalt alloy, nickel-cobalt base alloy, nickel-titanium alloy, stainless steel, and titanium). The mid-shaft hypotube 502 may comprise an interrupted spiral cut pattern. FIG. 5A shows a similar view as FIG. 4A, but with the rail subassembly 21 removed, thereby exposing the mid-shaft subassembly 22.

Similar to the other subassemblies, the mid-shaft hypotube 502 and/or mid-shaft proximal tube 504 can comprise a tube, such as a hypodermic tube or hypotube (not shown). The tubes can be made from one of any number of different materials including Nitinol, stainless steel, and medical grade plastics. The tubes can be a single piece tube or multiple pieces connected together. Using a tube made of multiple pieces can allow the tube to provide different characteristics along different sections of the tube, such as rigidity and flexibility. The mid-shaft hypotube 502 can be a metal hypotube. The mid-shaft hypotube 502 can have a number of slots/apertures cut into the hypotube. In some examples, the cut pattern can be the same throughout. In some examples, the mid shaft hypotube 502 can have different sections having different cut patterns. The mid-shaft hypotube 502 can be covered or encapsulated with a layer of ePTFE, PTFE, or other material so that the outer surface of the mid-shaft hypotube 502 is generally smooth. At least a portion of a length of the mid-shaft proximal tube 504 may be covered with a heat shrink tubing or wrap.

The pusher 506 may be configured for radially retaining a portion of the implant (e.g., prosthesis) 30 in a compacted configuration, such as a proximal end of the implant 30. For example, the pusher 506 may be a ring or covering that is configured to radially cover the proximal end portion (e.g., suture eyelets portion) of the implant 30. The pusher 506 can also be considered to be part of the implant retention area 316, and may be at the proximal end of the implant retention area 316. The pusher 506 may comprise a frustoconical or cup shape that is riveted or fastened on its opposite sides to the distal end of the mid-shaft hypotube 502. The pusher 506 may be formed of PEEK material, ferrous material, platinum iridium, or other fluorogenic material to facilitate radiographic imaging. The mid-shaft subassembly 22 may be disposed so as to be fixed with respect to the handle. In some examples, the mid-shaft subassembly 22 may be individually slidable with respect to the other subassemblies. The mid-shaft adapter 505 may operably couple to an actuator or depth knob 920 (shown in FIG. 9A). The depth knob 920 may be utilized to effect ventricular/atrial movement of shafts of the elongate catheter within a heart. Additional structural and operational details of a mid-shaft subassembly 22, such as those described in connection with mid assemblies in U.S. Publication No. 2019/0008640 and U.S. Publication No. 2019/0008639, which are hereby incorporated by reference herein, may be incorporated into the mid-shaft subassembly 22.

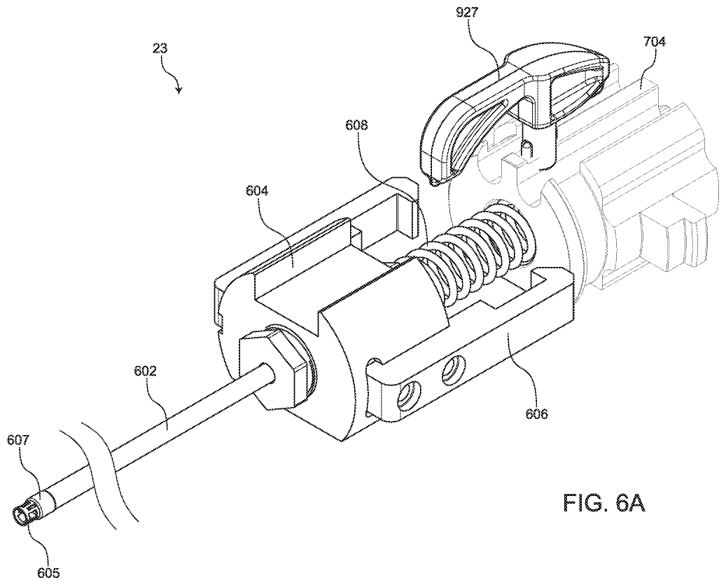

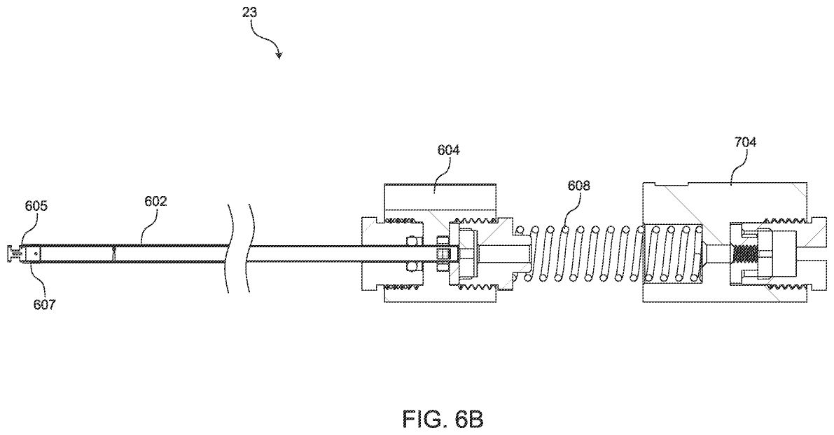

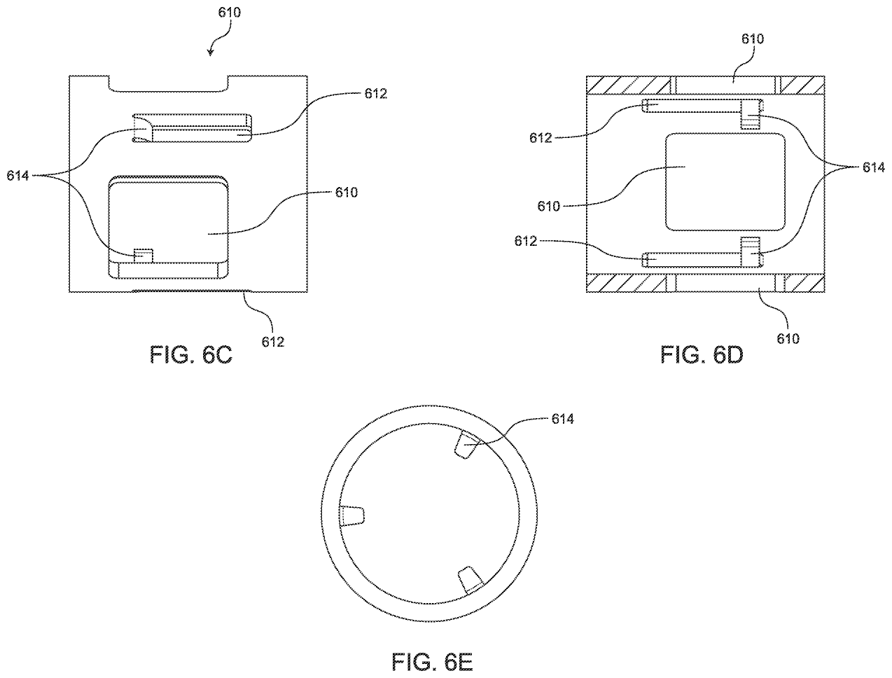

Moving radially inward from the mid-shaft subassembly 22, FIG. 6A shows a perspective view of a release shaft or subassembly 23 of the delivery device 15 of the delivery system 10. FIG. 6B shows a side cross-section view of the release subassembly 23 of FIG. 6A. The release subassembly 23 operates in conjunction with the manifold subassembly 24 to facilitate retention and release of the implant or prosthesis 30. The release subassembly 23 extends through a central lumen of the mid-shaft subassembly 22. The release subassembly 23 includes a release shaft 602 that includes a lumen. The manifold subassembly 24 extends through the lumen of the release subassembly 23. The mid-shaft subassembly 22 acts to prevent the implant 30 from retreating when the capsule subassembly 306 is pulled back, and the manifold subassembly 24 prevents distal valve movement.

The distal portion of the release shaft 602 may include laser cut portions having various spine patterns. For example, a distal-most portion (e.g., ˜1 cm) of the release shaft 602 may include a dual spine laser cut pattern and a portion proximal of the distal-most portion (e.g., ˜5 cm proximal of the distal-most portion) may include a universal laser cut spine pattern. The dual spine pattern portion may only travel through the primary distal flex portion of the rail hypotube 406 and the universal spine pattern portion may travel through both the primary and secondary flex portions of the rail hypotube 406. At least a portion of a length of the release shaft 602 may be surrounded by a heat-shrink wrap or liner. The proximal end of the release shaft 602 is operably coupled to the handle 14 (e.g., via release adapter 604 at a proximal portion of the release shaft 602). The release subassembly 23 also includes a distal release tip 605 coupled to a distal end of the release shaft 602 via a coupler 607, which may be formed of PEBAX or other thermoplastic elastomer material. The distal release tip 605 may be welded to the distal end of the release shaft 602. The release adapter 604 includes release snaps 606 on opposite lateral sides. The release snaps 606 engage with a distal portion of the manifold adapter 704 after release of the tethers or sutures so as to prevent movement of the manifold subassembly 24 and release subassembly 23 with respect to each other, which could cause the windows 610 (marked in FIG. 6C) of the distal release tip 605 to close and inadvertently retain one of the sutures or tethers. Thus, the release snaps 606 convert the release/manifold mechanism from a normally-closed configuration to an open configuration and allows the manifold subassembly 24 and release subassembly 23 to track proximally together. The release subassembly 23 further includes a release spring 608 that extends between the release adapter 604 and a location within a manifold adapter 704 of the manifold subassembly 24.

FIGS. 6C, 6D, and 6E show a close-up side view, side cross-section view, and bottom view, respectively, of the distal release tip 605. The distal release tip 605 cooperates in conjunction with a distal end portion of the manifold subassembly 24 to facilitate prevention of premature release of the implant 30 and to facilitate release (e.g., untethering) of the implant 30 when ready for final implantation. The distal release tip 605 includes three windows 610 spaced apart around a circumference of the distal release tip 605 and three slots 612, with each slot 612 positioned between two adjacent windows 610. The three windows 610 may be equally spaced apart circumferentially and the slots 612 may be positioned equally circumferentially between adjacent windows 610. A distal end of each of the slots 612 includes an inwardly-protruding retention member 614 (e.g., tab, protrusion, lock, anchor). The inwardly-protruding tabs 614 are adapted to be aligned with and extend within corresponding slots of the manifold subassembly 24 so as to control axial movement and to prevent rotation of the release assembly 23 with respect to the manifold subassembly 24, as will be described in more detail below.

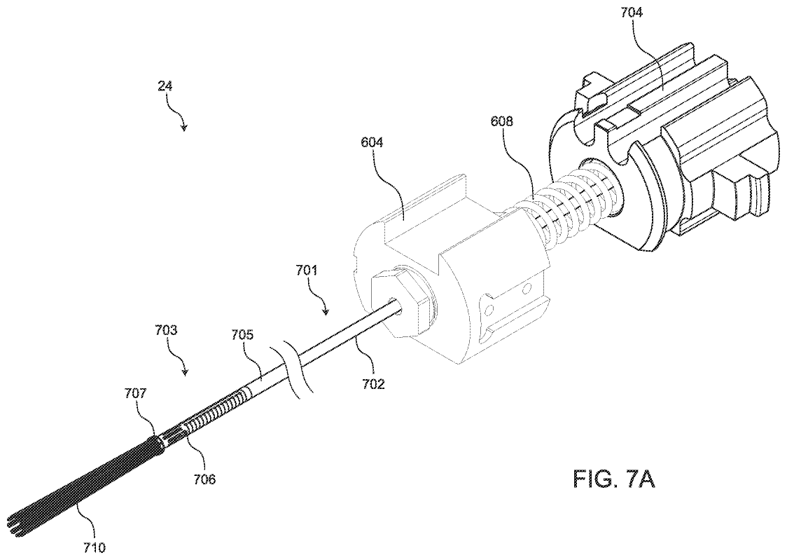

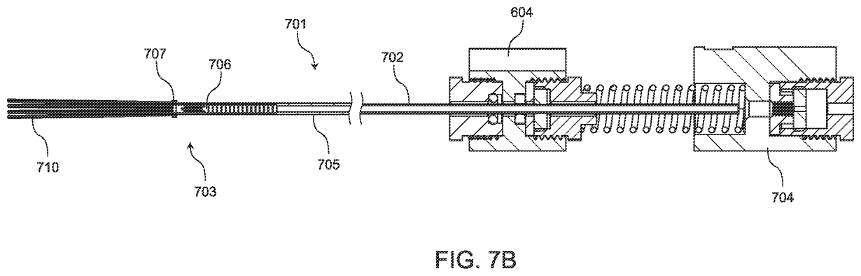

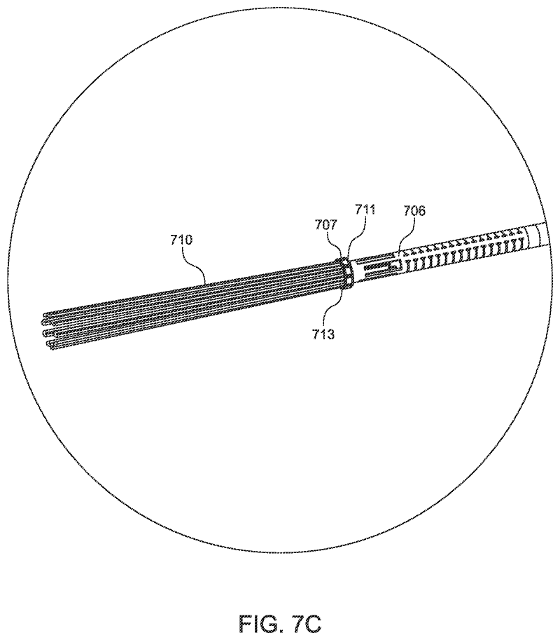

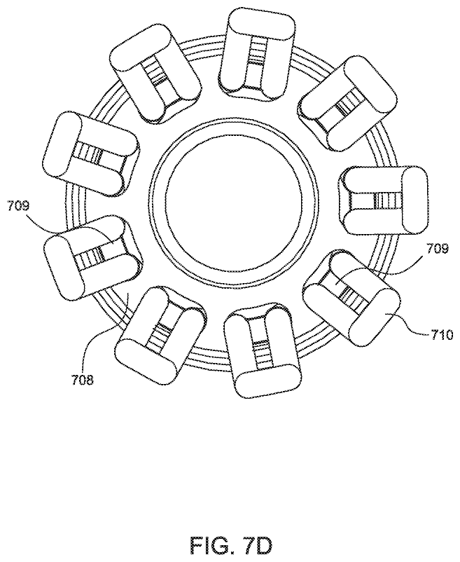

Moving radially inward, FIG. 7A shows a perspective view of the manifold shaft or subassembly 24 of the elongate catheter or delivery device 15. FIG. 7B shows a side cross-section view of the manifold subassembly 24 of FIG. 7A. The manifold subassembly 24 extends through and along the lumen of the release subassembly 23. The manifold subassembly 24 includes a proximal subassembly 701 and a distal subassembly 703. The proximal subassembly 701 includes a proximal shaft 702 having a proximal end that extends into the handle 14 of the delivery device 15 and is operably coupled to the handle 14 via a manifold adapter 704 at a proximal portion of the manifold shaft or subassembly 24. The proximal shaft 702 may be coupled to the distal subassembly 703 by a manifold cable 705. The manifold cable 705 may comprise a multi-layer cable comprised of two, three, four, five or more layers. In some implementations, the manifold cable 705 comprises a tri-layer cable in which two outer layers function for tension and act together to prevent unwrapping of the outer layers and an inner layer comprises a single-filar coil that provides compression and prevents collapse. In some implementations, each layer is wound in an opposite direction as the adjacent layer (e.g., clockwise, counter-clockwise, clockwise or counter-clockwise, clockwise, counter-clockwise). The wire size, wire tension, pitch, number of filars in each layer, material, and material properties may vary. An inner coil may comprise one to ten filars closely wound with a 0 to 0.005″ gap. The middle and outer coils may each comprise one to ten filars and be closely wound with a 0 to 0.010″ gap. The manifold cable 705 may be formed of one or more materials, including, for example, nitinol, ferrous material such as stainless steel, and/or cobalt chrome material. The temper (e.g., strength) of the wires may range from 100 KSI to 420 KSI. The cross-section of the wires may be flat or round. The tri-layer cable may be configured to prevent diameter change during stretching. In other implementations, the proximal shaft 702 extends all the way to and is bonded with a proximal end of the distal subassembly 703.

FIG. 7C shows a close-up view of the distal subassembly 703 of the manifold subassembly 24. FIG. 7D shows a bottom view of the distal subassembly 703 of the manifold subassembly 24. As shown, the distal subassembly 703 includes a proximal tether retention component 706 and a distal tether retention component 707. The distal tether retention component 707 may be coupled (e.g., permanently bonded, welded) to a distal end of the proximal tether retention component 706. As shown best in FIG. 7D, the distal tether retention component 707 may comprise a cog that includes outwardly-extending tether cleats 708 circumferentially spaced around the cog. Openings or gaps 709 exist between adjacent tether cleats 708 to receive portions of the tether or suture 710. The distal tether retention component 707 may include proximal and distal seal members 711, 713 (e.g., retention rings) that are sealed (e.g., welded, glued or otherwise adhered) to opposite upper and lower sides of the distal tether retention component 707 during manufacture to seal off the openings or gaps 709 between the tether cleats 708 so as to prevent the tether or suture 710 from being removed or uncoupled from the distal tether component 707. In accordance with several examples, the tether 710 is intended to be permanently coupled to (i.e., non-removable from) the distal tether retention component 707. The number of tether cleats 708 may correspond to the number of eyelets on the implant 30 (e.g., upper eyelets of the outer frame 34). The number of tether cleats 708 is nine in the illustrated example; however, other numbers of tether cleats 708 may be used.

The tether or suture 710 may be a continuous piece of tether or suture that forms offset proximal loops and distal loops along its continuous length upon assembly during manufacturing. The proximal loops are wrapped around the tether cleats 708 and the distal loops are fed through a respective eyelet on a proximal end of the implant or prosthesis 30 (e.g., upper eyelet of an outer frame 34) and then removably coupled to the delivery device 15 (e.g., the proximal tether retention component 706 of the manifold subassembly 24).

During assembly, the continuous tether or suture 710 may be coupled to the distal tether retention component 707 according to the following example implementation. One end of the continuous tether or suture 710 may start at a location spaced distal to the distal tether retention component 707. With the one end remaining there, the tether 710 is then wrapped around a first tether cleat 708 and then fed back through an opening or gap 709 on the other side of the first tether cleat 708 to form a first proximal loop and then brought back to a location spaced distal to the distal tether component 707 to start formation of a first distal loop. The process is repeated for each of the tether cleats 708 until all of the proximal and distal loops are formed and the second end of the continuous tether 710 is brought near the first end of the continuous tether 710 and the two ends are knotted together and bonded to form a single continuous strand. The tether assembly process may be facilitated by an assembly component that can be placed at an appropriate spacing distance distal of the distal tether retention component 707 and that includes pegs around which portions of the continuous tether 710 can be wrapped to form the distal loops at uniformly-spaced distances from the distal tether component 707. The proximal loops may be prevented from unhooking from the tether cleats 708 by the proximal and distal seal members 711, 713.

FIG. 7E shows a flat cut pattern of the proximal tether retention component 706 of the distal subassembly 703. As shown, the proximal portion of the proximal tether retention component 706 comprises a dual spine laser cut pattern. The dual spine laser cut pattern of the proximal tether retention component 706 may match a dual spine laser cut pattern of the rail subassembly 21 and the release subassembly 23. The distal end portion of the proximal tether retention component 706 comprises three circumferentially spaced slots 714 and three openings or windows 715. The slots 714 are configured to align circumferentially with the slots 612 of the distal release tip 605 and the openings or windows 715 are configured to align circumferentially with the windows 610 of the distal release tip 605. Other numbers of slots 714 and openings 715 (e.g., two, four, five, six, seven, eight, nine) may also be used in other examples. Each opening 715 includes a tab, finger, or peg, 716 extending a certain distance into a respective opening 715 from a distal edge of the respective opening 715. A length of each tab 716 is sufficient such that one or more distal tether loops can be looped over a top (or proximal end) of a respective tab 716 and pushed distally so as to retain the one or more distal tether loops. As shown, the three tabs 716 each have a different length in order to facilitate the initial tether assembly process. Each tab 716 may receive one or more distal tether loops. In one implementation where there are nine distal tether loops, each tab 716 may retain three distal tether loops. The slots 714 may be equally circumferentially spaced around a longitudinal axis of the proximal tether retention component 706 and may be sized and spaced so as to align with corresponding slots 612 of the release subassembly 23 so as to receive a respective inwardly-protruding retention member 614.

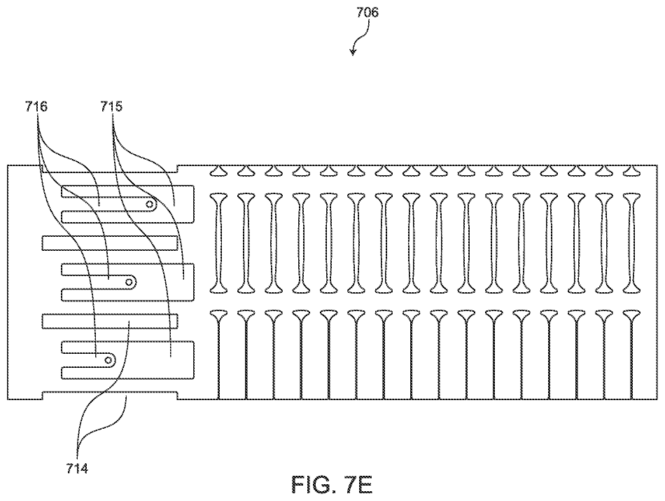

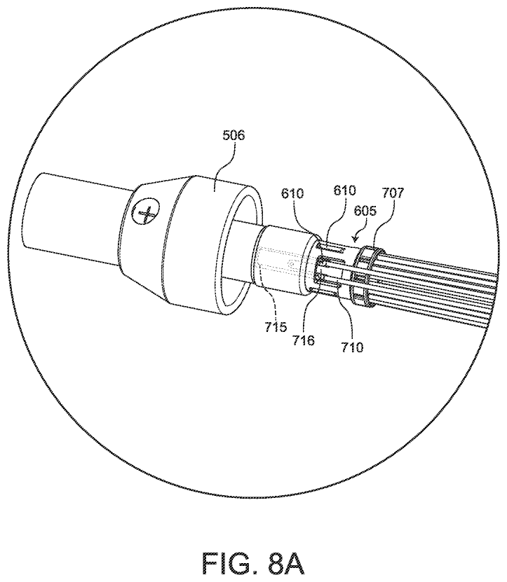

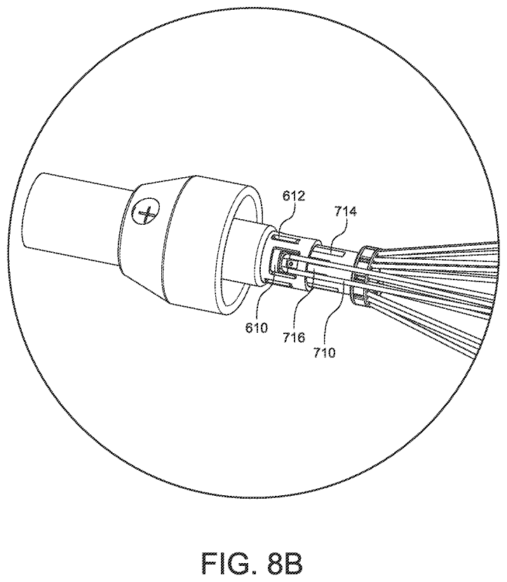

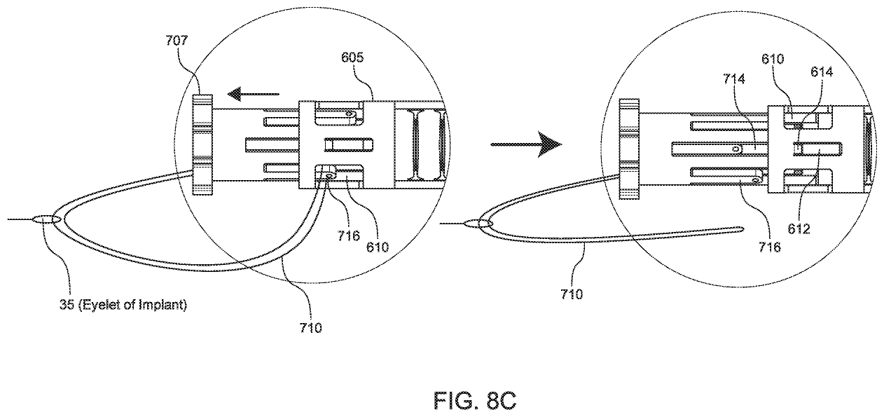

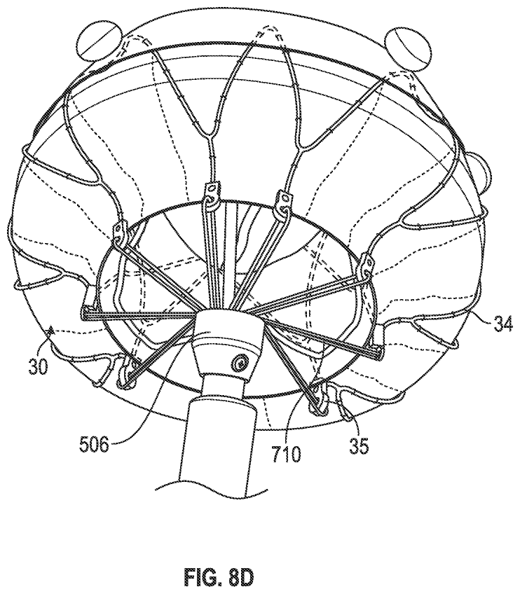

FIGS. 8A and 8B show distal end portions of the release and manifold shafts or subassemblies in a locked configuration and unlocked configuration, respectively. The locked configuration shown in FIG. 8A is the default configuration after assembly. The release and manifold subassemblies are intended to remain in the locked configuration until a clinician has determined that the implant 30 is in a final desired implantation location. In the locked configuration, the proximal ends of the tabs 716 are positioned proximal of the proximal edge of the release windows 610 such that the distal tether loop(s) wrapped around the tabs 716 cannot be unhooked from the tabs 716, which could cause premature release of the tether 710. For simplicity, only one distal tether loop is shown wrapped around one of the tabs 716; however, two, three, or more tether loops may be hooked onto, or wrapped around, each of the tabs 716. The spring 608 shown in FIG. 6A (which is biased in a compressed configuration) keeps the release adapter 604 and the manifold adapter 704 apart and forces the release subassembly 23 distal in compression, thereby keeping the release subassembly 23 and the manifold subassembly 24 in the locked configuration shown in FIG. 8A. As discussed in connection with FIGS. 9A and 9B, a safety member (e.g., pin) 927 also prevents the manifold shaft or subassembly 24 from moving distally out of the locked configuration until ready.

Once the clinician has determined that the implant 30 is in a final desired implantation position and all verification processes have been performed and confirmed, the safety member 927 is removed and the spring 608 is placed even more in compression. As the actuator knob or release knob 925 is rotated distally, the spring 608 is compressed further and pushes the manifold shaft or subassembly 24 distally out of the release subassembly 23 into the unlocked configuration shown in FIG. 8B. As shown in FIG. 8B, the manifold subassembly 24 has been pushed distally enough that the proximal end of at least one of the tabs 716 is within the release window 610 such that a distal tether loop of the tether 710 can be unhooked from the tab 716, especially upon continued distal advancement of the manifold subassembly 24.

FIG. 8C illustrates how one of the tether or suture loops transitions from being tethered to being untethered, or released, as the release and manifold subassemblies effect transition between a locked configuration and an unlocked configuration. Also as shown in FIG. 8C, the corresponding slots 612 and 714 are aligned so as to prevent rotation of the manifold subassembly 24 with respect to the release subassembly 23 (due to inwardly-protruding retention members 614), thereby retaining alignment of the tabs 716 within the windows 610 of the release subassembly 23.

FIG. 8D shows an implant 30 fully tethered between cyclets on a proximal end of the implant (e.g., upper cyclet of an outer frame 34 of a valve prosthesis 30) and the manifold subassembly 24 of the delivery device 15. As shown, there are nine tether loops or portions connected to nine eyelets; however, the number may vary as desired and/or required. The suture or tether retention mechanism described herein advantageously does not require the tethers or sutures 710 to extend through and along a long portion of the length of the delivery device 15.

FIG. 9A shows a perspective view of the housing or handle 14 of the delivery device 15. FIG. 9B shows a side cross-section view of the handle 14. The handle 14 includes a control mechanism for moving the one or more shafts of the elongate catheter. The control mechanism may include multiple actuators, such as rotatable knobs, that can manipulate different components (e.g., cause movement of respective shafts or subassemblies of the shaft assembly 12) of the delivery system 10. The distal end of the handle 14 includes an actuator in the form of a capsule knob 905. Rotation of the capsule knob 905 in one direction causes proximal movement of the outer sheath subassembly 20 in an axial direction so as to unsheathe and deploy a distal portion (e.g., ventricular portion) of the implant 30 from the capsule subassembly 306. Rotation of the capsule knob 905 in the opposite direction causes distal movement of the outer sheath subassembly 20 (including the capsule subassembly 306) so as to recapture, retrieve, or resheath, the implant 30 within the capsule subassembly 306. The outer sheath subassembly 20 may be individually translated with respect to the other subassemblies in the delivery device 15. The distal end of the implant 30 can be released first, while the proximal end of the implant 30 can remain radially compressed within the pusher 506 of the mid-shaft subassembly 22.

Moving proximally, the handle 14 includes a stabilizer mounting area 910 adapted to interface with a clamp of a stabilizer assembly 1100 configured to control the medial/lateral position of the delivery device 15. Moving further proximally are the actuators in the form of primary flex rail knob 915A and the secondary flex rail knob 915B. Rotation of the primary flex rail knob 915A causes flexing of the primary flex portion, or distal slotted hypotube section 406D of the rail hypotube 406 to effect changes in medial/lateral trajectory. Rotation of the secondary flex rail knob 915B causes flexing of the primary flex portion, or proximal slotted hypotube section 406P of the rail hypotube 406 to effect changes in anterior/posterior trajectory. However, the number of flex rail knobs 915A, B can vary depending on the number of pull wires used.

Proximal to the secondary flex rail knob 915B is the depth knob 920 that controls movement of the outer sheath subassembly 20, the mid-shaft subassembly 22, the release subassembly 23 and the manifold subassembly 24 relative to the rail subassembly 21. The depth knob 920 may also move the other subassemblies together as well relative to the rail subassembly 21 in some configurations.

Further proximal is the actuator in the form of the actuator knob or release knob 925. The release knob 925 may be rotated proximally to put tension on the manifold subassembly 24 during loading or during recapture, or retrieval, of the implant 30. The release knob 925 may be rotated distally to deploy the proximal portion (e.g., atrial portion) of the implant 30 (after the capsule subassembly 306 has been retracted) to deploy the distal portion (e.g., ventricular portion) of the implant 30. Distal movement of the release knob 925 takes tension off the manifold subassembly 24. As discussed above, the safety stop member 927 prevents the release knob 925 from moving distally enough to allow release of the implant 30 until the safety stop member 927 is removed from the handle 14. Once the safety stop member 927 has been removed, continued distal movement of the release knob 925 causes the manifold subassembly 24 to move distally relative to the release subassembly 23 to facilitate release of the tether 710 from the manifold subassembly 23 (e.g., the distal tether loops are allowed to be pushed off of the tabs 716 of the proximal tether retention member 706 of the manifold subassembly 23 by the windows 610 of the release assembly 23). The proximal-most knob is the nose cone knob 930, rotation of which causes proximal and distal movement of the nose cone subassembly.

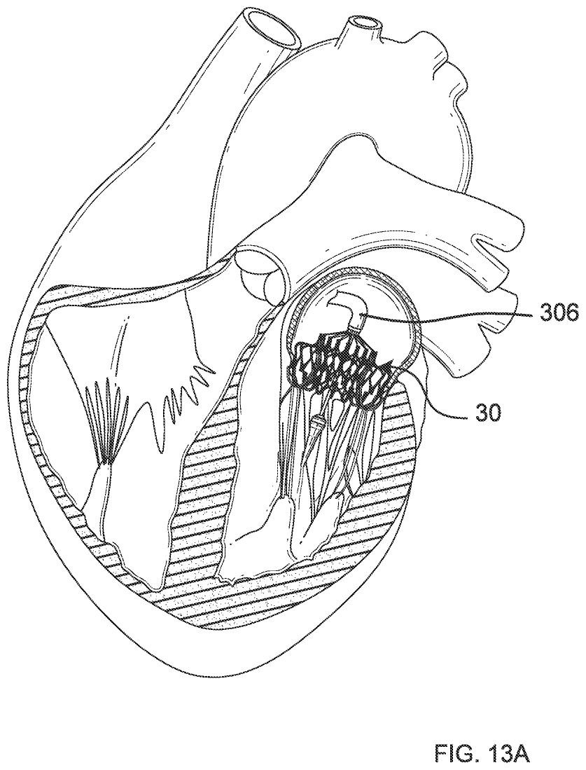

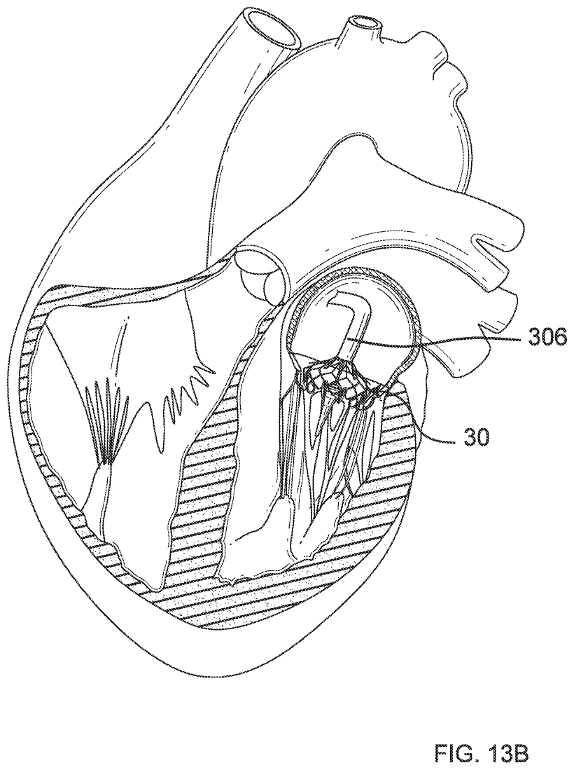

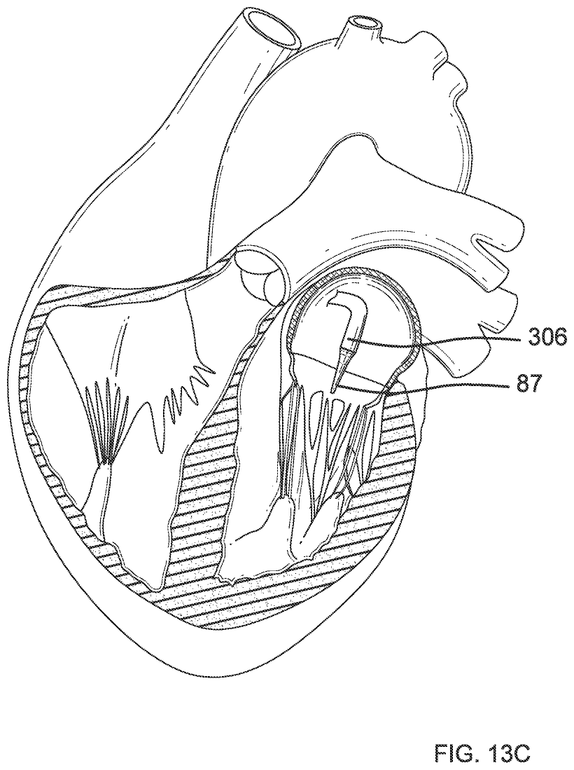

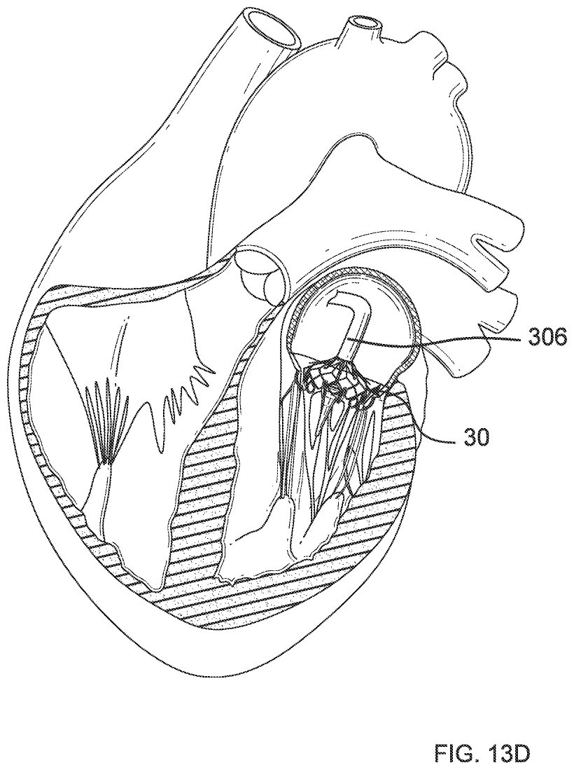

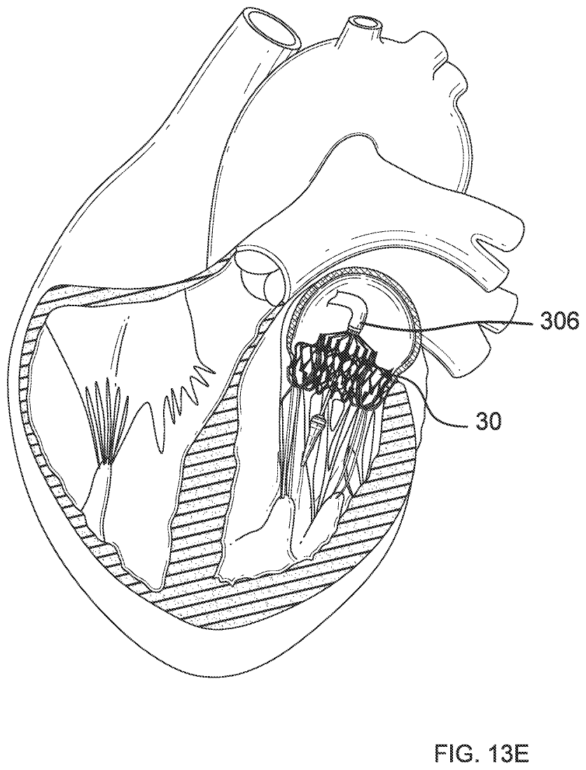

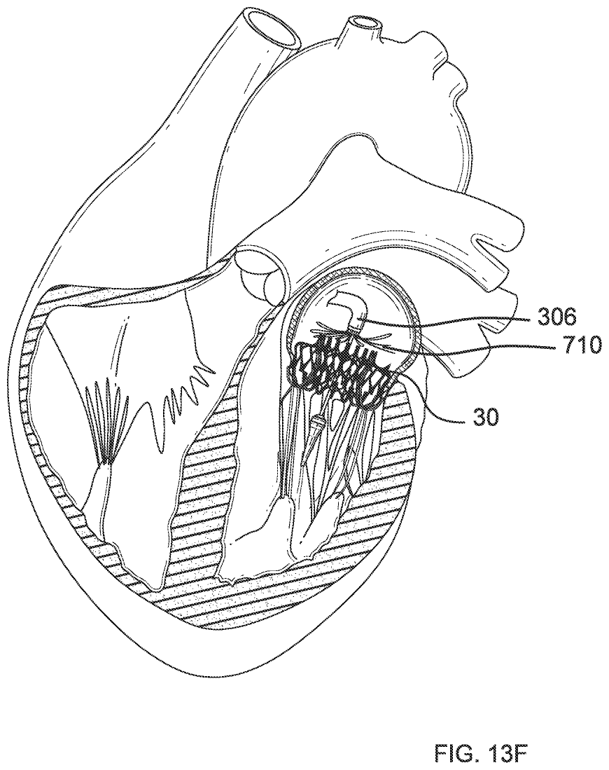

The nose cone subassembly is the most radially-inward subassembly and may include a nose cone shaft having a distal end connected to a nose cone 87 (labeled in FIG. 13C). For example, the knob 930 can be a portion of the nose cone subassembly that extends from a proximal end of the handle 14. Thus, a user can pull or push on the knob 930 or rotate the knob 930 to translate the nose cone shaft distally or proximally individually with respect to the other shafts. This can be advantageous for proximally translating the nose cone 87 into the outer sheath assembly 20/capsule subassembly 306, thus facilitating withdraw of the delivery device 15 from the patient. The nose cone 87 can have a tapered tip. The nose cone 87 can be made of thermoplastic or elastomer or material such as PEBAX® for atraumatic entry and to minimize injury to venous vasculature. The nose cone 87 can also be radiopaque to provide for visibility under fluoroscopy. The nose cone assembly is preferably located within a lumen of the manifold subassembly 24. The nose cone assembly can include a lumen for a guide wire to pass therethrough. Additional structural and operation details of a handle and a nose cone assembly, such as those described in connection with handles and nose cone assemblies in U.S. Publication No. 2019/0008640 and U.S. Publication No. 2019/0008639, which are hereby incorporated by reference herein, may be incorporated into the handle 14 and nose cone subassembly herein.

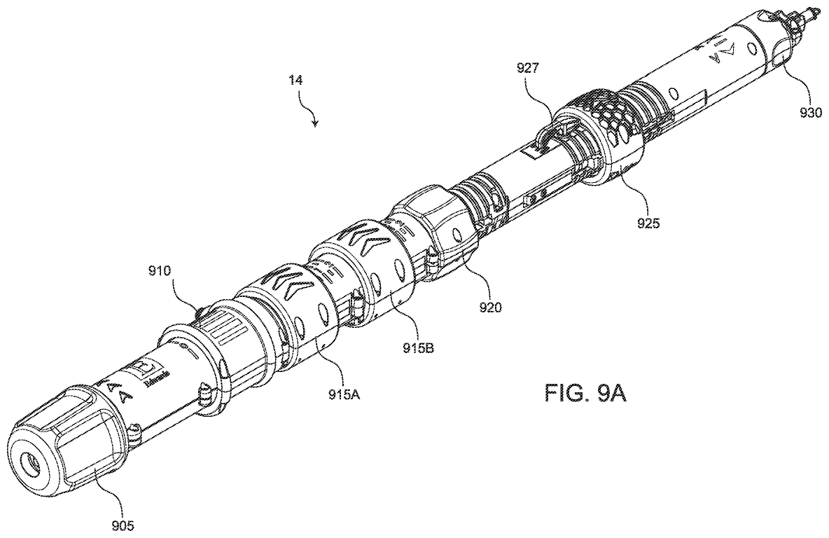

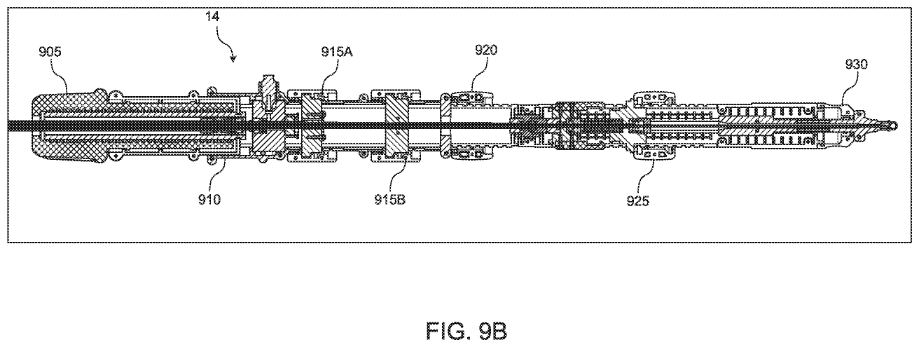

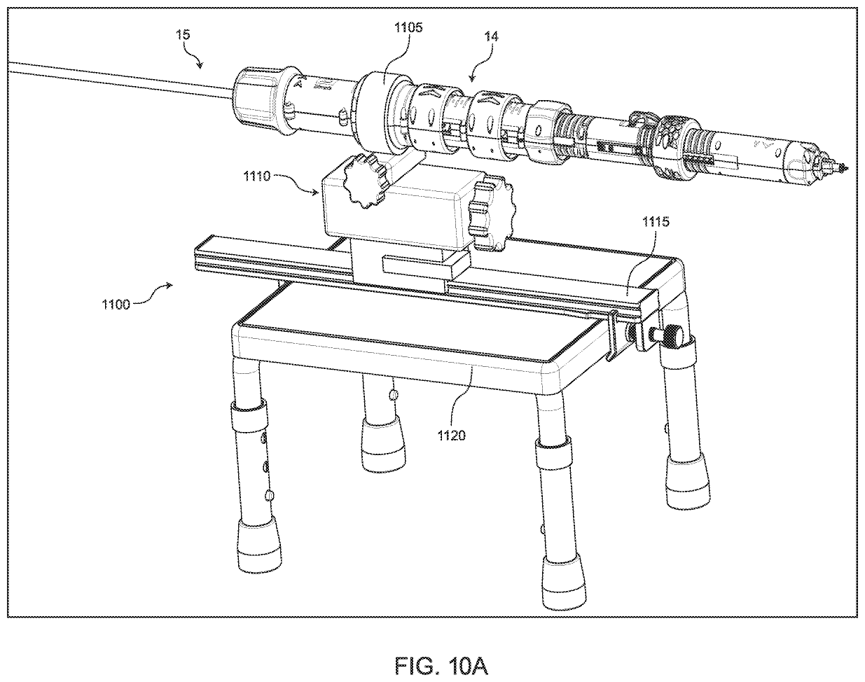

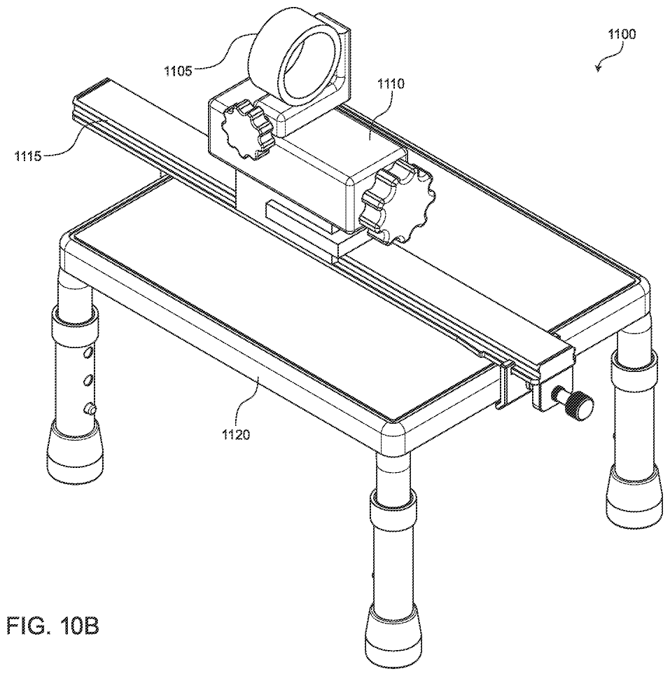

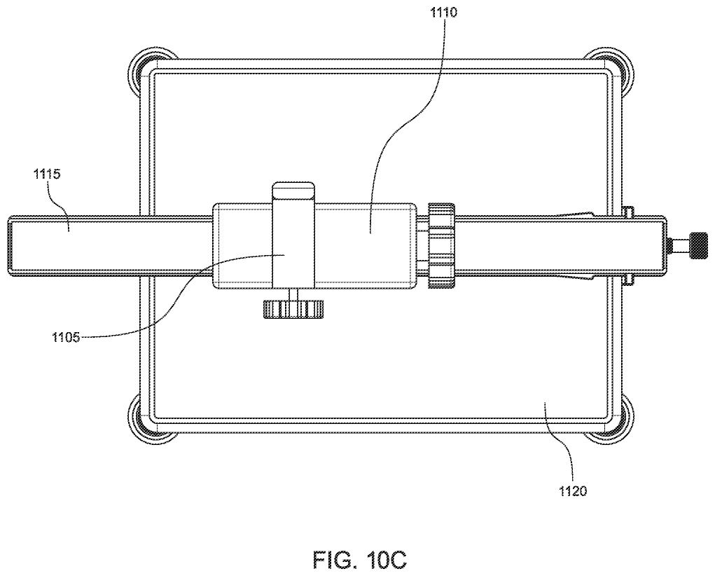

FIG. 10A illustrates how the handle 14 of the elongate catheter or delivery device 15 interfaces with an example of the stabilizer assembly 1100 of the delivery system 10. FIG. 10B shows a perspective view of the stabilizer assembly 1100 without the delivery device 15 attached. FIG. 10C shows a top view of the stabilizer assembly 1100 of FIG. 10A. The stabilizer assembly 1100 includes a clamp 1105, a guide assembly 1110, a rail 1115, and a base 1120. The clamp 1105 is configured to couple to the stabilizer mounting area 910 of the handle 14 of the delivery device 15. The guide assembly 1110 is configured to cause changes in the medial/lateral position of the delivery device 15 by movement along the rail 1115. The rail 1115 may be mounted on and secured to the base 1120. Additional details regarding the stabilizer assembly 1100 may be found in US Pat. Publ. No. 2020/0108225 published on Jan. 10, 2020, the entire contents of which are incorporated by reference herein.

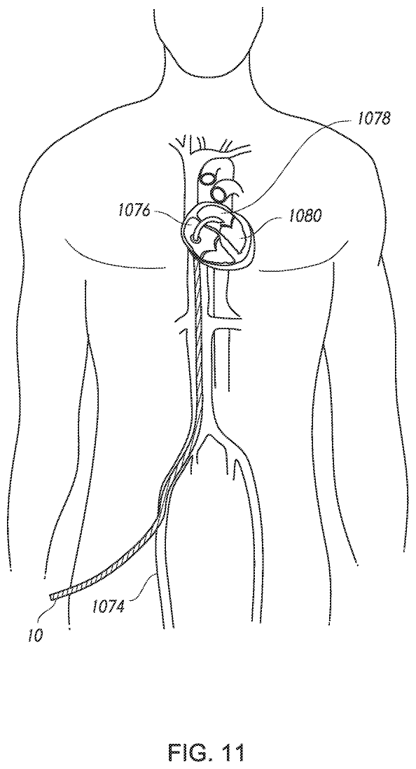

FIG. 11 illustrates a schematic representation of a transseptal delivery approach. As shown in FIG. 11, in one example the delivery system 10 can be placed in the ipsilateral femoral vein 1074 and advanced toward the right atrium 1076. A transseptal puncture using known techniques can then be performed to obtain access to the left atrium 1078. The delivery system 10 can then be advanced into the left atrium 1078 and then to the left ventricle 1080. FIG. 11 shows the delivery system 10 extending from the ipsilateral femoral vein 1074 to the left atrium 1078. In example of the disclosure, a guide wire is not necessary to position the delivery system 10 in the proper position, although in other examples, one or more guide wires may be used.

Accordingly, it can be advantageous for a user to be able to steer the delivery system 10 through the complex areas of the heart in order to position a replacement mitral valve in line with the native mitral valve. This task can be performed with or without the use of a guide wire with the above disclosed system. The distal end of the delivery system 10 can be advanced into the left atrium 1078. A user can then manipulate the rail subassembly 21 to target the distal end of the delivery system 10 to the appropriate area. A user can then continue to pass the bent delivery system 10 through the transseptal puncture and into the left atrium 1078. A user can then further manipulate the delivery system 10 to create an even greater bend in the rail subassembly 21. Further, a user can torque the entire delivery system 10 to further manipulate and control the position of the delivery system 10. In the fully bent configuration, a user can then place the replacement valve in the proper location. This can advantageously allow delivery of a replacement valve to an in-situ implantation site, such as a native mitral valve, via a wider variety of approaches, such as a transseptal approach.

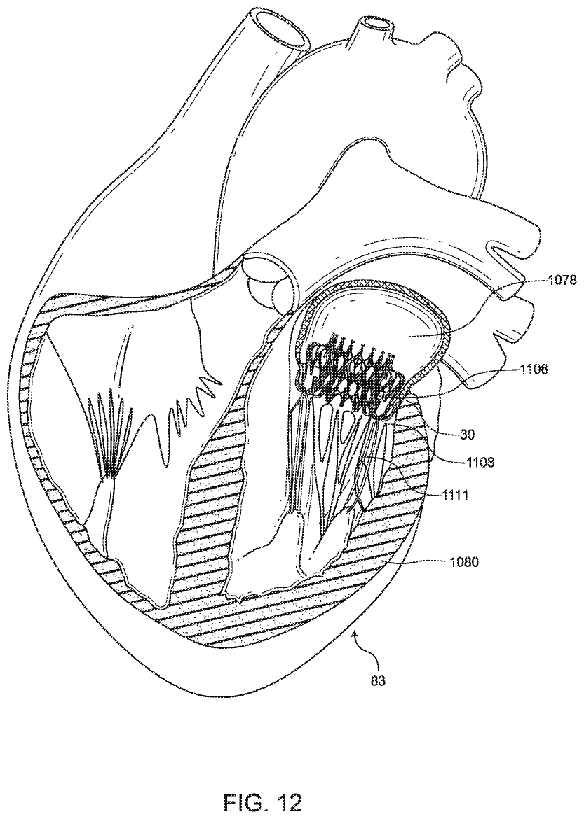

FIG. 12 illustrates a schematic representation of a portion of an example of a replacement heart valve (implant 30) positioned within a native mitral valve of a heart 83. Further details regarding how the implant 30 may be positioned at the native mitral valve are described in U.S. Pat. Pub No. 2015/032800 published on Nov. 19, 2005, the entirety of which is hereby incorporated by reference, including but not limited to FIGS. 13A-15 and paragraphs [0036]- [0045]. A portion of the native mitral valve is shown schematically and represents typical anatomy, including a left atrium 1078 positioned above an annulus 1106 and a left ventricle 1080 positioned below the annulus 1106. The left atrium 1078 and left ventricle 1080 communicate with one another through the annulus 1106. Also shown schematically in FIG. 12 is a native mitral leaflet 1108 having chordae tendineae 1111 that connect a downstream end of the mitral leaflet 1108 to the papillary muscle of the left ventricle 1080. The portion of the implant 30 disposed upstream of the annulus 1106 (toward the left atrium 1078) can be referred to as being positioned supra-annularly. The portion generally within the annulus 1106 is referred to as positioned intra-annularly. The portion downstream of the annulus 1106 is referred to as being positioned sub-annularly (toward the left ventricle 1080).

As illustrated in FIG. 12, the implant 30 can be positioned so that the ends or tips of the distal anchors 37 are on a ventricular side of the mitral annulus 1106. The distal anchors 37 can be positioned such that the ends or tips of the distal anchors 37 are on a ventricular side of the native leaflets beyond a location where chordac tendineae 1111 connect to free ends of the native leaflets. The distal anchors 37 may extend between at least some of the chordae tendineae 1111 and, in some situations can contact or engage a ventricular side of the annulus 1106. It is also contemplated that in some situations, the distal anchors 37 may not contact the annulus 1106, though the distal anchors 37 may still contact the native leaflet 1108. In some situations, the distal anchors 37 can contact tissue of the left ventricle 1080 beyond the annulus 1106 and/or a ventricular side of the leaflets 1108.

FIGS. 13A-13F illustrate various steps of deployment and recapture of the implant (e.g., replacement heart valve) 30 using the elongate catheter or delivery device 15 described herein. The capsule subassembly 306 advantageously facilitates recapture of the implant 30 after an initial deployment. FIG. 13A illustrates an initial deployment of the implant 30 from the elongate catheter delivery device 15. For example, the initial deployment may be within a mitral valve annulus following a transfemoral and/or transseptal approach. Note that the implant 30 remains tethered to the elongate catheter or delivery device 15 upon initial full deployment of the implant 30 to a fully expanded configuration. In some instances, a clinician may decide after performing various tests (e.g., using various imaging modalities and measurements) that the initial deployment location is not ideal. For example, the ideal position may be more superior or inferior of the initial deployment location. In order to prevent damage to the implant 30 and to the heart, the implant 30 may be recaptured prior to movement of the implant 30 to a new implantation location. Recapturing of the implant 30 may be performed by advancing the capsule subassembly 306 of the outer sheath subassembly 20 distally over the implant 30 to cause the implant 30 to transition to a compressed configuration.

FIGS. 13B and 13C show various stages of recapturing of the implant 30. As shown in FIG. 13B, the capsule subassembly 306 has been advanced distally to capture the proximal portion of the implant 30. FIG. 13C shows full recapture of the implant 30, with the capsule subassembly 306 being fully advanced distally until contact with a nose cone 87 of the nose cone subassembly.

After movement of the distal end of the delivery device 15 to a new location, the capsule subassembly 306 of the outer sheath subassembly 20 can again be retracted proximally to unsheathe the distal portion of the implant 30 (e.g., at a new implantation location within a mitral valve annulus or tricuspid valve annulus), as shown in FIG. 13D. The manifold and release subassemblies 23, 24 may then be advanced distally to deploy the proximal portion of the implant 30 out of the pusher 506 of the mid-shaft subassembly 22, as shown in FIG. 13E. After confirmation that the fully-deployed implant 30 is in an ideal and proper final implantation location, the tether 710 may be caused to be released from the manifold subassembly 24 as shown in FIG. 13F and the delivery device 15 can be retracted and removed from the heart and then from the subject altogether.

During a deployment procedure, it may be advantageous to reduce a tension force or a compression force in at least one of the shafts of the elongate catheter or delivery device 15. For example, during a deployment procedure, one of the shafts (such as the shaft of the outer sheath subassembly 20) may be retracted to provide a tension force in the shaft. Similarly, one of the shafts may be advanced to provide a compression force in the shaft (such as the shaft of the outer sheath subassembly 20). Actions that may produce a tension force in a shaft such as the outer sheath subassembly 20 may comprise a retraction of the capsule subassembly 306 during an implant release procedure. Actions that may produce a compression force in a shaft such as the outer sheath subassembly 20 may comprise a distal advancement of the capsule subassembly 306 during an implant recapture procedure.

Similarly, a tension or compression force in one of the shafts may produce a tension or compression force in another of the shafts. A force from one shaft may place load on one or more other shafts of the elongate catheter or delivery device 15. For example, a tension force in the outer sheath subassembly 20 may produce a tension force in another subassembly such as the manifold subassembly 24. Too great of a tension force in the manifold subassembly 24 may impede the ability of the manifold subassembly 24 to operate properly or in instances may damage the manifold subassembly 24. It thus may be beneficial to reduce a tension force or a compression force in at least one of the shafts to reduce the force in that shaft, and also to reduce the tension force or compression force in another shaft of the elongate catheter. Such reduction may reduce damage to a shaft.

Methods of reducing a tension force or a compression force in a shaft may include manually operating a control mechanism (such as a handle knob as disclosed herein) to reduce the tension force or compression force in the shaft. The control mechanism may control all or fewer of the total number of shafts of the elongate catheter (e.g., one or more of the shafts). For example, referring to FIG. 9A, the capsule knob 905 may be rotated manually to retract the capsule subassembly 306, which may produce a tension force in the capsule subassembly 306. To reduce such a tension force, a user may rotate the capsule knob 905 in an opposite direction to “back drive” or “back off” the retraction of the capsule subassembly 306 by an amount to reduce the residual tension force within the shaft. The user may reduce the tension force such that the tension in the capsule subassembly 306 may be zero or neutral with respect to the other shafts of the delivery device 15 or elongate catheter. A similar operation of “back drive” or “back off” may occur when the capsule subassembly 306 is advanced in a recapture procedure. The user may “back drive” or “back off” the capsule subassembly 306 by rotating the capsule knob 905 in an opposite direction to reduce the amount of residual compression force within the shaft.

Producing a manual “back drive” or “back off,” however, may be laborious for the user. Further, the user may forget to produce the manual “back drive” or “back off,” which may leave residual tension or compression force within a shaft and may produce damage or inoperability of one or more shafts of the elongate catheter. For example, a tension force in the outer sheath subassembly 20 may render it difficult to operate the rail subassembly 21 and may produce undue tension in the manifold subassembly 24, which may damage the tethers or sutures 710 coupled to the implant, the manifold cable 705, or another component of the manifold subassembly 24. Various other adverse effects may result.

As such, a force reduction mechanism may be provided that may be configured to automatically reduce a tension force or a compression force in at least one of the one or more shafts of the elongate catheter. The force reduction mechanism may be for automatically reducing a tension force or a compression force in at least one of the one or more shafts upon a respective threshold tension force or threshold compression force being met in the at least one of the one or more shafts. The force reduction mechanisms disclosed herein may be configured to reduce a tension or compression force to lower the tension or compression force, entirely eliminate a tension or compression force, and/or equalize a tension or compression force across multiple shafts of the elongate catheter. Forces across a plurality of shafts may be equalized or neutralized in examples.

The force reduction mechanisms disclosed herein may be configured to reduce the tension or compression force in a shaft that is being moved by a control mechanism, or in another shaft of the elongate catheter that is not moved by the control mechanism yet experiences a tension or compression force. The tension or compression force of the shaft of the elongate catheter that the force reduction mechanism may reduce may be produced by external body forces or other forces applied to the shaft during deployment, or may be caused by the movement or tension or compression of another shaft of the elongate catheter. For example, a movement of the outer sheath subassembly 20 via a control mechanism may produce a tension force in the outer sheath subassembly 20 that may produce a corresponding tension in the manifold subassembly 24. The force reduction mechanisms may be configured to reduce the tension force in one or both of the outer sheath subassembly 20 or the manifold subassembly 24 produced by movement of the outer sheath subassembly 20. The force reduction mechanisms may be configured to automatically allow the at least one of the one or more shafts to move distally to reduce a tension force produced by the one or more shafts being moved proximally by the control mechanism. Similarly, the force reduction mechanisms may be configured to automatically allow the at least one of the one or more shafts to move proximally to reduce a compression force produced by the one or more shafts being moved distally by the control mechanism.

The force reduction mechanism may be configured to automatically reduce the tension or compression force of a shaft being moved by the control mechanism, or in a different shaft that is not moved by the control mechanism. For example, upon the control mechanism moving a first shaft, the force reduction mechanism may automatically reduce the tension force or compression force in the first shaft. Upon the control mechanism moving a first shaft, the force reduction mechanism may automatically reduce the tension force or compression force in a second shaft that may or may not be moved by the control mechanism. Various other shafts or subassemblies of the elongate catheter may have a tension or compression force reduced according to the force reduction mechanisms disclosed herein.

In examples, reducing a force in a shaft such as an outer sheath subassembly 20 or a manifold subassembly 24 may provide improved operation of other shafts of the elongate catheter or delivery device 15. For example, improved operation of the rail subassembly 21 may occur when forces are reduced in other shafts of the elongate catheter or delivery device 15. Improved flexibility of the rail subassembly 21 may result. Improved operation of multiple shafts of the elongate catheter or delivery device 15 may occur when forces across shafts are neutralized.

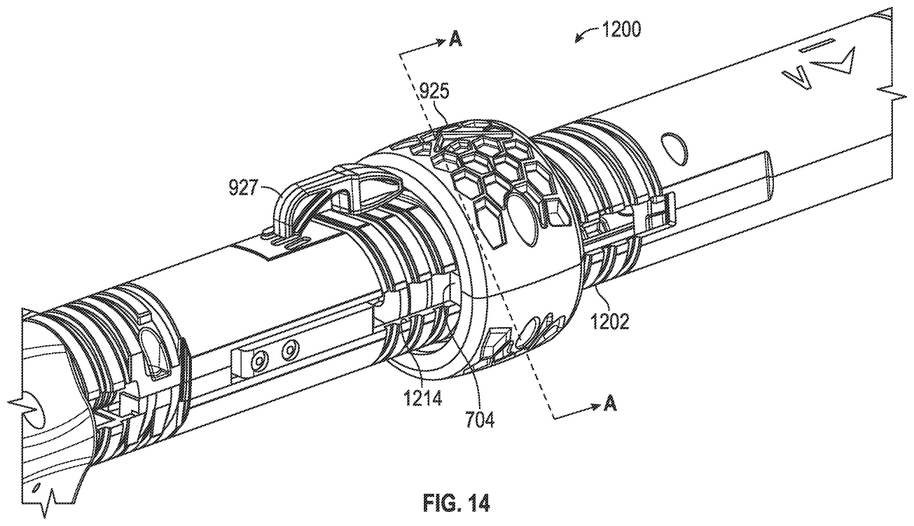

FIG. 14, for example, illustrates an example of a force reduction mechanism 1200 configured to allow a control mechanism actuator in the form of the release knob 925 to automatically advance distally or retract proximally upon a respective tension force or compression force being applied to the manifold subassembly 24. The force reduction mechanism 1200, for example, may be configured to automatically allow the release knob 925 to rotate relative to the handle to reduce the tension force or the compression force in the manifold subassembly 24. The release knob 925 may be configured to be rotated about threading 1202 in a direction to advance the manifold adapter 704 distally (for release of the tethers or sutures 710) or may be rotated in an opposite direction to retract the manifold assembly 704 proximally (for securement of the tethers or sutures 710). For example, the safety stop member 927 shown in FIG. 14 may be rotated to an unlocked position to allow the release knob 925 to be rotated to move the manifold adapter 704.

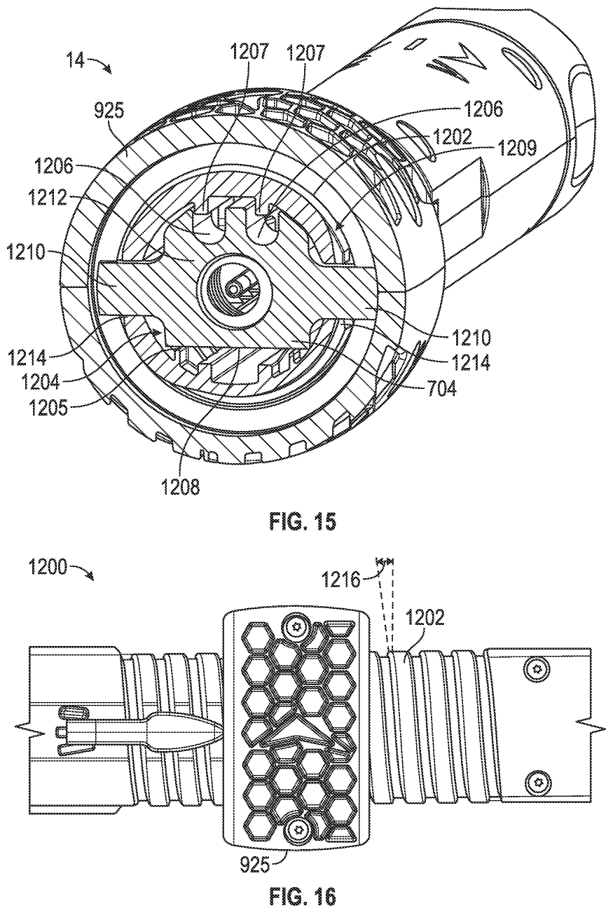

FIG. 15 illustrates a cross sectional view of the handle along line A-A shown in FIG. 14. The handle 14 may include an interior lumen 1204 for the manifold adapter 704 to be positioned within. The handle 14 may include an interior flattened surface 1205 for the manifold adapter 704 to slide longitudinally along. The handle 14 may include protrusions 1207 for passing into channels 1206 of the manifold adapter 704. The handle 14 may include openings 1214 or channels extending from the interior surface of the handle 14 to an outer surface 1209 of the handle 14. The openings 1214 may extend longitudinally along the handle 14. The outer surface 1209 of the handle 14 may include the threading 1202.

The manifold adapter 704 may be positioned within the interior lumen 1204 of the handle 14 and may be at a proximal portion of the manifold subassembly 24. The manifold adapter 704 may include guides in the forms of channels 1206 that may be configured to receive the protrusions 1207 of the handle 14. The manifold adapter 704 may include a flattened portion 1208 that may slide along the interior flattened surface 1205 of the handle 14. The flattened portion 1208 and the channels 1206 may serve to prevent rotation of the manifold adapter 704 within the interior lumen 1204 of the handle upon a longitudinal movement of the manifold adapter 704.

The manifold adapter 704 may further include a central body 1212 and engagement surfaces 1210 that may extend radially outward from the central body 1212. The engagement surfaces 1210 may comprise one or more protrusions or wings, or may have another form, that may allow for engagement with the release knob 925. The one or more protrusions or wings may extend radially outward from the manifold adapter 704 for engaging the release knob 925. The engagement surfaces 1210 may be configured to pass through the openings 1214 or channels in the handle and may slide longitudinally along the openings 1214 or channels.

The release knob 925 may be configured to rotate about the threading 1202 on the outer surface 1209 of the handle 14 and may engage with the engagement surfaces 1210 of the manifold adapter 704. The release knob 925 accordingly may move the manifold adapter 704 longitudinally due to the engagement between the engagement surfaces 1210 and the release knob 925. The ability of the manifold adapter 704 to slide along the handle 14 yet not rotate may allow the manifold adapter 704 to convert the rotational motion of the release knob 925 to linear or longitudinal motion of the manifold adapter 704 and the manifold subassembly 24.

If a tension or compression force were experienced in the manifold subassembly 24, the tension or compression force may be transmitted directly to the manifold adapter 704 in the form of a respective distal or proximal force upon the manifold adapter 704. The tension or compression force may thus be transmitted through the engagement surfaces 1210 to the release knob 925. As such, a release knob 925 able to rotate may reduce the tension or compression force within the manifold subassembly 24.

Referring to FIG. 16, a force reduction mechanism 1200 may comprise a configuration of the threading 1202 and/or the release knob 925 that may automatically allow for a “back drive” or “back off” of the release knob 925 upon an axial load (e.g., a tension or compression force) being applied to the release knob 925. Features of the force reduction mechanism 1200 that may allow for an automatic “back drive” or “back off” may comprise the screw lead, and the reverse efficiency. The following equation represents a back driving torque (T_{b}) (in Newton-meters) as a function of the axial load (F) (in Newtons), the screw lead (P) (in meters), and the reverse efficiency (η₂).

$${T}_{b} = \frac{( F ) ( P ) ( η 2 )}{2 π}$$

The screw lead, and the reverse efficiency, or a combination of these factors may be utilized to produce a desired “back drive” or “back off” of the release knob 925. The threading 1202, for example, may have a pitch angle 1216 that produces a screw lead that may allow for automatic rotation of the release knob 925 relative to the handle upon an axial load, to relieve the tension force or compression force within the manifold subassembly 24. As such, upon a user releasing the user's hand from the release knob 925, the release knob 925 may automatically rotate to reduce the tension force or compression force within the manifold subassembly 24.

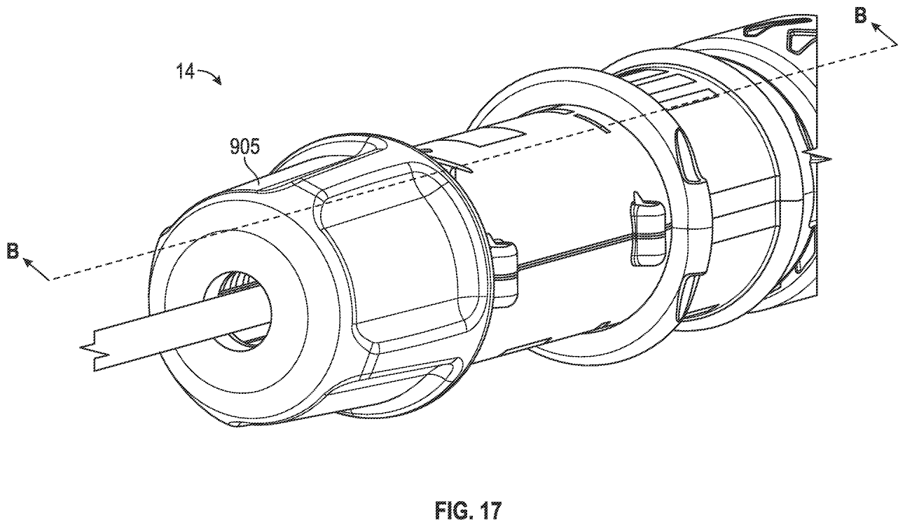

The use of an actuator configured to automatically rotate to reduce the tension force or compression force within a subassembly is not limited to the manifold subassembly 24, and may be utilized with any subassembly disclosed herein. For example, FIG. 17 illustrates a view of the distal portion of the handle 14, and particularly the capsule knob 905.

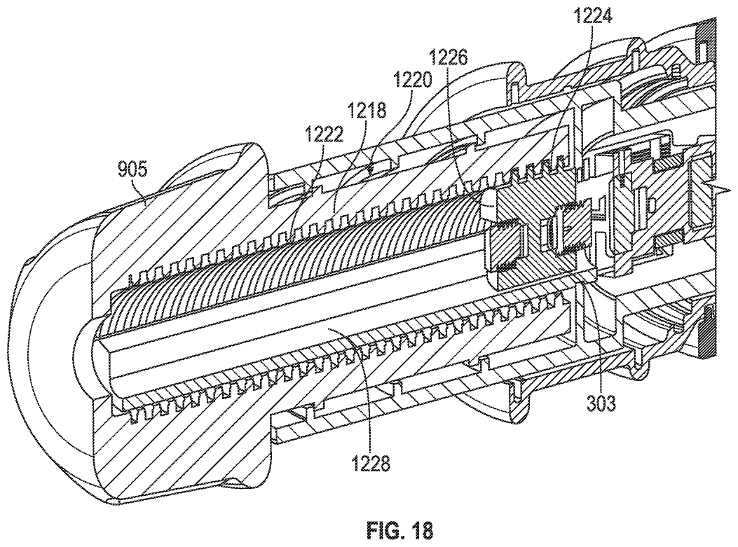

FIG. 18 illustrates a cross sectional view of the handle 14 along line B-B in FIG. 17. The handle 14 includes an interior lumen 1220 that extends longitudinally. A beam 1228 may extend within the interior lumen 1220 and may have a “U” shape (more clearly shown in FIG. 37).

The capsule knob 905 is shown to couple to a rotating body 1218 that may extend longitudinally along the interior lumen 1220 of the handle 14. The rotating body 1218 may have a cylindrical shape and may include an interior surface having threading 1222. The threading 1222 may engage corresponding threading 1224 on the outer sheath adapter 303.

The outer sheath adapter 303 may be positioned at a proximal portion of the outer sheath subassembly 20 and may include a guide in the form of a flange 1226 that extends outward from the outer sheath adapter 303 and contacts the beam 1228 within the interior lumen 1220. The “U” shape of the beam 1228 may prevent rotation of the outer sheath adapter 303. The outer sheath adapter 303, being restrained from rotational movement, may slide longitudinally or linearly along the beam 1228 upon the rotation of the rotating body 1218 due to the engagement of the threading 1222, 1224.

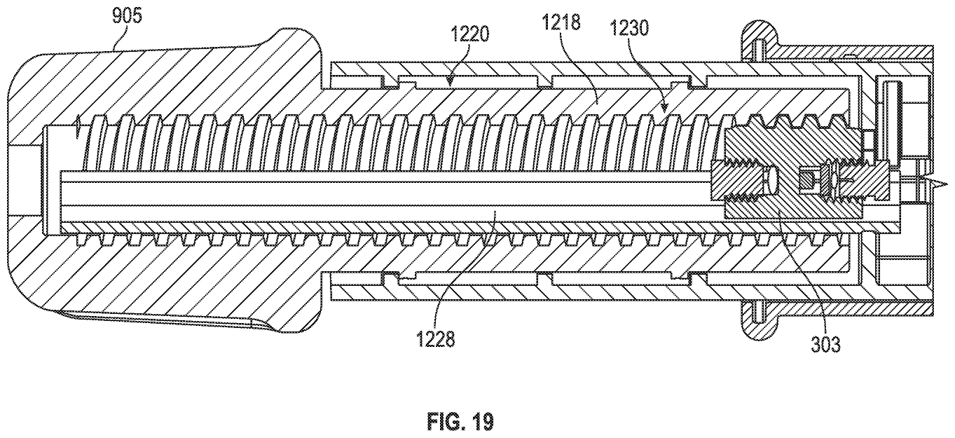

FIG. 19, for example, illustrates a side cross sectional view of the distal portion of the handle 14 along line B-B shown in FIG. 17. Similar to the force reduction mechanism 1200 of FIG. 16, a force reduction mechanism 1230 may be provided to similarly allow the capsule knob 905 to rotate to automatically reduce the tension or compression of the outer sheath. Similar to the force reduction mechanism 1200 of FIG. 16, the screw lead, and the reverse efficiency, or a combination of these factors may be utilized to produce a desired “back drive” or “back off” of the capsule knob 905. Various other actuators of the delivery system may be configured to produce a desired “back drive” or “back off.”

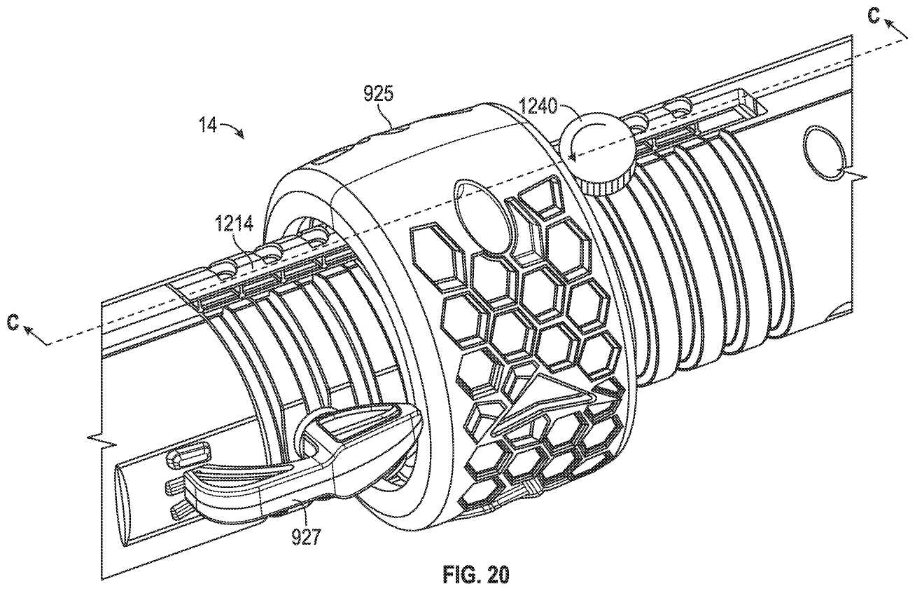

In examples, components may be provided to adjust the reverse efficiency (η₂). FIG. 20, for example, illustrates a friction component that may be utilized to vary the reverse efficiency (η₂). The friction component may comprise a clamp 1240 that may be placed along the handle 14 to vary the reverse efficiency for the release knob 925.

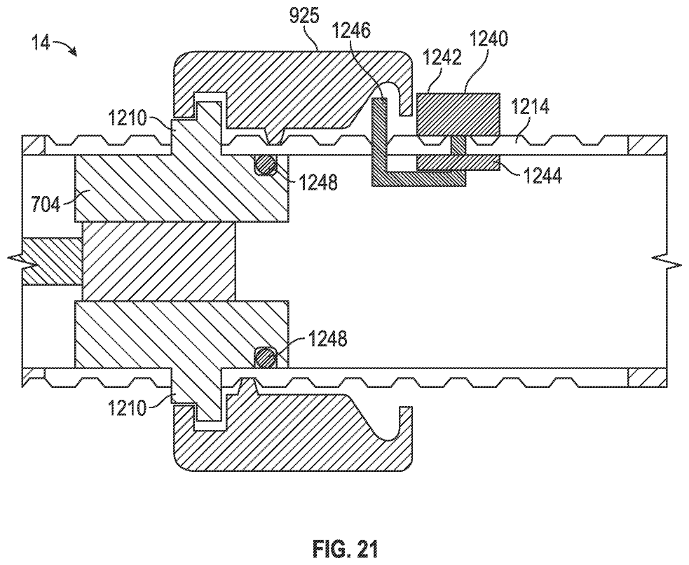

FIG. 21, for example, illustrates a cross sectional view of the handle along line C-C shown in FIG. 20. The clamp 1240 is shown to pass through the opening 1214 with an outer clamp body 1242 positioned exterior of the handle 14 and an inner clamp body 1244 positioned interior of the handle 14. The outer clamp body 1242 may comprise a rotatable body, configured to be tightened with respect to the inner clamp body 1244 to increase a friction force between the clamp 1240 and the handle 14. The variation in friction force may be controlled with the tightness of the outer clamp body 1242. The clamp 1240 may include an engagement portion 1246 configured to engage the release knob 925 and convey the increased friction provided by the clamp 1240 to the release knob 925.

In examples, the amount of friction, and accordingly the variation in the reverse efficiency (12), and the amount of “back drive” or “back off” may be controlled by adjustment of the clamp 1240. For example, tightening the clamp 1240 may increase the friction, and loosening the clamp 1240 may decrease the friction. A user accordingly may control the reverse efficiency (η₂) to either increase or decrease the responsive movement or rotation of the release knob 925 according to an axial load placed upon the manifold subassembly, which may be a proximal or compressive axial load or a distal or tension axial load.

Other friction components may be utilized in combination or solely. For example, friction components such as friction rings 1248 or O-rings may be provided that may vary the reverse efficiency (η₂). The friction rings 1248 may be provided to slide along an interior surface of the handle 14, or along another portion of the elongate catheter 15. The friction rings 1248 may be positioned upon the manifold adapter 704 or may have another position as desired.

The use of one or more friction components may occur with the manifold subassembly 24 or may occur with any other subassembly or shaft disclosed herein. Features discussed in regard to FIGS. 16-21 may be utilized with a manifold shaft or manifold subassembly 24, or may be utilized with any other shaft or assembly or subassembly of a delivery system. For example, the examples of FIGS. 16-21 may be implemented with an outer sheath or outer sheath subassembly 20, or any other shaft or assembly or subassembly disclosed herein. The rail subassembly 21, mid-shaft subassembly 22, release subassembly 23, or nose cone assembly may utilize the examples of FIGS. 16-21. Any feature of a control mechanism, such as a control knob disclosed herein may be utilized in combination or utilized in alternative with the examples of FIGS. 16-21. Any feature of an adapter disclosed herein may be utilized in combination or utilized in alternative with the examples of FIGS. 16-21.

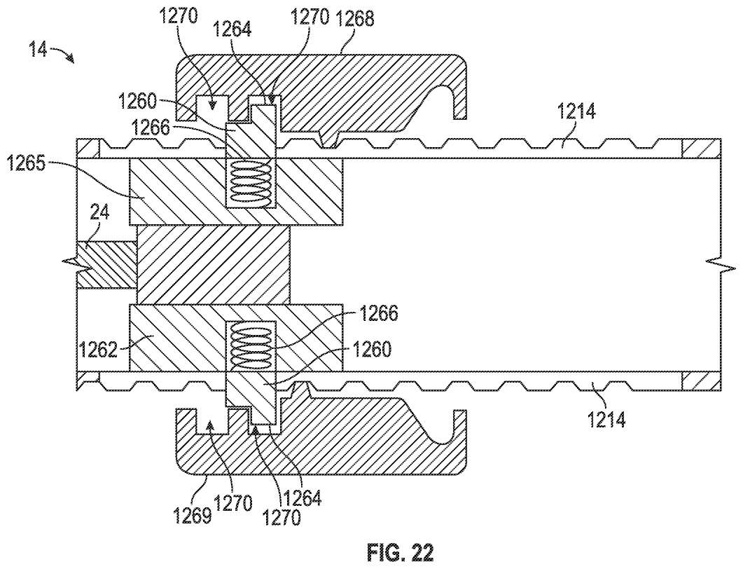

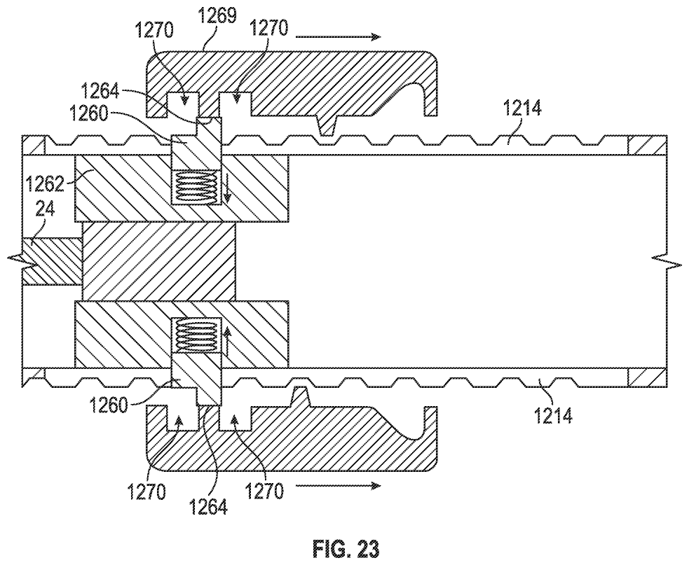

Other forms of force reduction mechanisms configured to automatically reduce a tension force or a compression force in a shaft may be utilized. FIGS. 22-23, for example, illustrate an implementation in which the force reduction mechanism may be configured to automatically reduce the tension force or the compression force in a shaft by allowing the shaft to move proximally or distally relative to a control mechanism. A displacement body 1260 may be utilized that may allow the shaft to disengage from the control mechanism.

FIG. 22 illustrates a cross sectional view of the handle 14 along a mid line such as line C-C in FIG. 20. The configuration of the handle 14 has been varied from the configuration shown in FIG. 20, although features of the configuration shown in FIG. 20 may be utilized with the features of FIG. 22. The manifold adapter 1262 has been provided with engagement surfaces 1264 in the form of displacement bodies 1260 configured to displace inward upon a sufficient axial force being applied to the manifold adapter 1262. Displacement bodies configured to allow the manifold subassembly 24 to move proximally or distally relative to the control mechanism 1268 may further comprise springs 1266 that may bias the displacement bodies 1260 outward from a central body 1265 of the manifold adapter 1262. The springs 1266 may be positioned on the manifold adapter 1262. The springs 1266 may press the displacement bodies 1260 of the manifold adapter 1262 towards the actuator in the form of an actuator knob or release knob 1269.

The actuator knob or release knob 1269 may be for advancing or retracting the shaft or manifold subassembly 24, with the actuator knob or release knob 1269 engaged with the shaft or manifold subassembly 24.

The engagement surfaces 1264 may comprise one or more wings or protrusions, similar to the engagement surfaces 1210 shown in FIG. 15, yet may be configured displace inward due to the compressibility of the springs 1266. The one or more wings or protrusions may extend radially outward from the manifold adapter 1262 for engaging the actuator knob or release knob 1269.

The control mechanism 1268 may comprise an actuator in the form of the actuator knob or release knob 1269 that may include one or more engagement portions 1270 that may be configured to engage the engagement surfaces 1264. The actuator knob or release knob 1269 may include an interior surface for engaging the engagement surfaces 1264. An engagement portion 1270 may comprise a recess in an interior surface of the release knob 1269 configured to receive the engagement surface 1264 of the manifold adapter 1262, or may have another configuration as desired.

The displacement bodies 1260 may be configured to allow the manifold subassembly 24 to move proximally or distally relative to the control mechanism 1268 to automatically reduce the tension force or the compression force in the at least one of the one or more shafts.

When an axial load threshold for the manifold adapter 1262 is reached, the springs 1266 may be compressed inward and allow the displacement bodies 1260 to displace inward. The displacement may disengage the engagement surfaces 1264 of the adapter 1262 from the actuator in the form of the release knob 1269 and allow the manifold adapter 1262 to slide axially relative to the release knob 1269. Such axial movement may reduce a tension force or a compression force in the manifold subassembly 24 for example.

The force reduction mechanism may allow the shaft or manifold subassembly 24 to disengage from the actuator knob or release knob 1269 for reducing a tension force in the shaft.

FIG. 23, for example, illustrates the manifold adapter 1262 having released from the release knob 1269. The springs 1266 may be compressed inward and the engagement surfaces 1264 may disengage from the engagement portions 1270 of the release knob 1269. The adapter may disengage from the actuator to automatically reduce the tension force or the compression force in at least one of the one or more shafts. The manifold adapter 1262 and the manifold subassembly 24 may be configured to slide relative to the release knob 1269. The force reduction mechanism may cause the shaft or manifold subassembly 24 to disengage from the actuator knob or release knob 1269 upon reaching a threshold tension force for allowing the shaft or manifold subassembly 24 to slide longitudinally relative to the actuator knob or release knob 1269.

In examples, the release knob 1269 may include multiple engagement portions 1270 that may be configured to engage the engagement surfaces 1264. The engagement portions 1270 may be placed adjacent to each other in series linearly along the release knob 1269. As such, the engagement surfaces 1264 may be configured to sequentially engage with the engagement portions 1270 upon a compression or tension force displacing the manifold adapter 1262 relative to the release knob 1269.

An indicator may be utilized that may indicate the tension force or the compression force in the at least one of the one or more shafts. For example, an auditory indicator may be utilized with the manifold adapter 1262 to indicate the displacement of the manifold adapter 1262 relative to the release knob 1269. The sequential engagement may produce a clicking sound such as “click-click,” or another sound that may indicate to a user that the manifold adapter 1262 is disengaging from the release knob 1269. Other forms of indicators may be utilized in examples herein. The displacement of the manifold adapter 1262 relative to the release knob 1269, for example, may produce a vibration that may comprise a haptic indicator.

Any subassembly disclosed herein may utilize a force reduction mechanism as disclosed in regard to FIGS. 22-23.

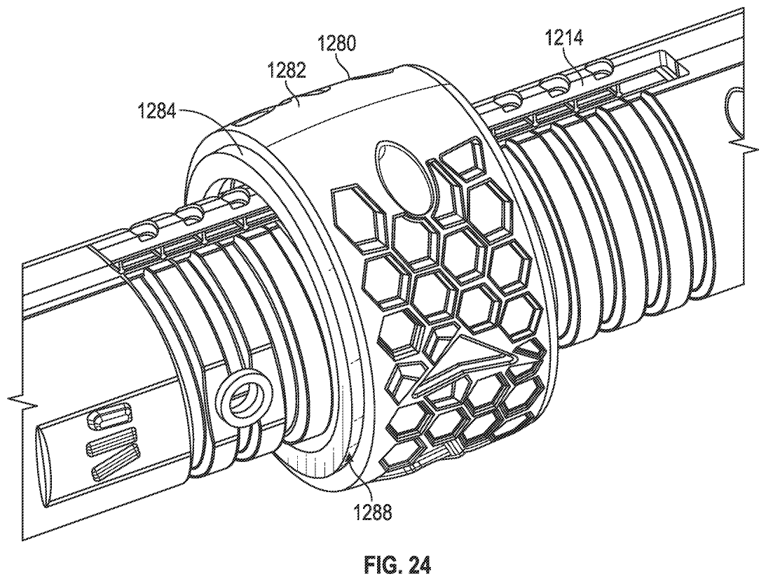

FIGS. 24-26 illustrate an example in which a portion of the release knob 1280 may displace relative to another portion of the release knob to reduce a tension force or compressive force within the manifold subassembly. Referring to FIG. 24, the release knob 1280 may include a first portion or outer portion 1282 and a second portion or inner portion 1284. The outer portion 1282, for example, may comprise a shell extending around the inner portion 1284. The outer portion 1282 may surround the inner portion 1284 or may have another configuration. The inner portion 1284 may be configured to displace relative to the outer portion 1282 upon a tension or compressive force being applied to the release knob 1280.

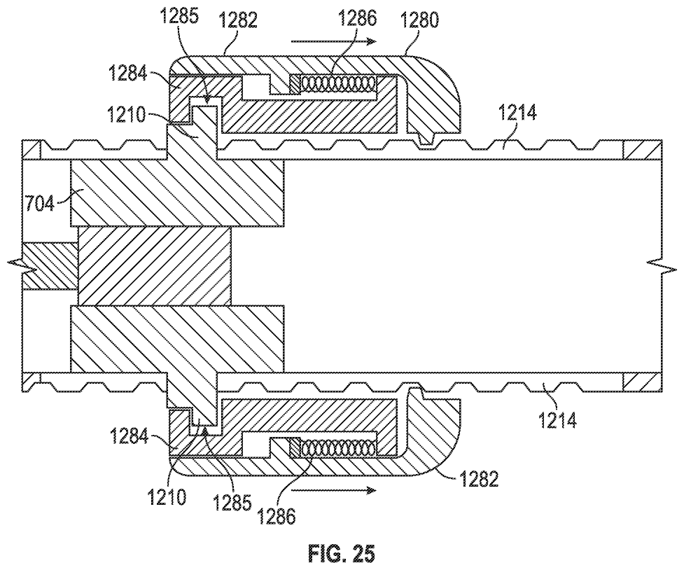

FIG. 25 illustrates a cross sectional view of the release knob 1280 along a mid line. The inner portion 1284 may engage the manifold adapter 704. The inner portion 1284, for example, may include engagement portions 1285 that may be configured to receive the engagement surfaces 1210 of the manifold adapter 704. The engagement portions 1285 may comprise a recess in an interior surface of the inner portion 1284 configured to receive the engagement surface 1210 of the manifold adapter 704, or may have another configuration as desired.

The inner portion 1284 may be configured to displace along with the manifold adapter 704 in response to a tension or compression force. A displacement body 1286 such as a spring may be positioned within the actuator in the form of the release knob 1280 and may be configured to bias the inner portion 1284 proximally towards the outer portion 1282. The displacement body 1286 may be configured to automatically allow the inner portion 1284 to displace relative to the outer portion 1282 upon a compression or tension force being applied to the manifold adapter 704, to reduce the tension or compression force in the manifold adapter 704. The displacement body 1286 may be configured to allow the manifold subassembly 24 to move proximally or distally relative to the control mechanism to automatically reduce the tension force or the compression force in the at least one of the one or more shafts.

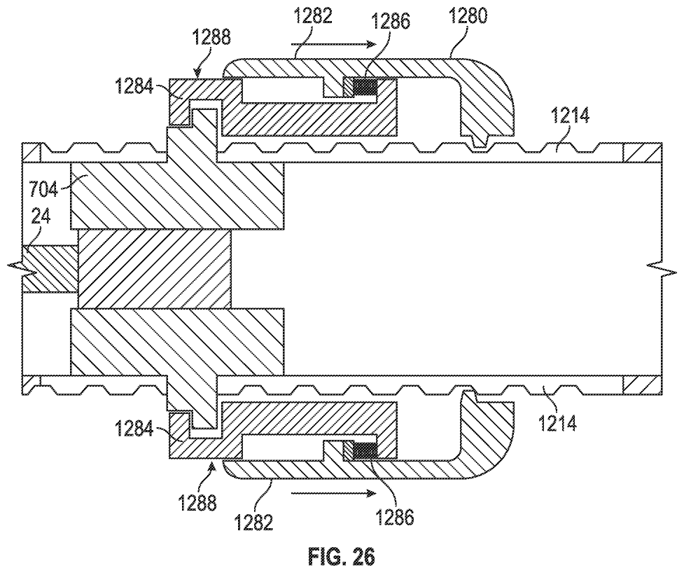

FIG. 26, for example, illustrates a tension force in the manifold adapter 704 having displaced the manifold adapter 704 distally. The inner portion 1284 may displace distally along with the manifold adapter 704. The displacement body 1286 may be compressed between the outer portion 1282 of the release knob 1280 and the inner portion 1284 of the release knob 1280.

In examples, the spring force of the displacement body 1286 may be set to determine an amount of force that causes the manifold adapter 704 and the inner portion 1284 to displace. For example, a lesser spring force may allow a lesser tension or compression force to cause displacement of the inner portion 1284 relative to the outer portion 1282. A greater spring force may allow a greater tension or compression force to cause displacement of the inner portion 1284 relative to the outer portion 1282.

In examples, the displacement of the outer portion 1282 relative to the inner portion 1284 may comprise a visual indicator of the tension or compression force applied to the manifold subassembly 24. The indicator may comprise the displacement of at least the body comprising the outer portion 1282 of the actuator from the body comprising the inner portion 1284 of the actuator, and the displacement of the adapter 704 from the outer portion 1282 of the release knob 1280. For example, an outer surface 1288 of the inner portion 1284 may comprise a visual indicator of the force applied to the manifold subassembly 24. The amount or length of the outer surface 1288 of the inner portion 1284 that is exposed from beneath the outer portion 1282 may indicate the amount of force. FIG. 24, for example, shows the exposure of the inner portion 1284 from the outer portion 1282. In examples, a scaling or other form of marker may be positioned on the outer surface 1288 to indicate the amount of force. For example, a greater amount of the scaling may be shown due to a greater amount or length of the outer surface 1288 being exposed based on the force applied to the manifold subassembly 24. The indicator may further comprise the displacement body 1286 in the form of the spring coupling the outer portion 1282 to the inner portion 1284.

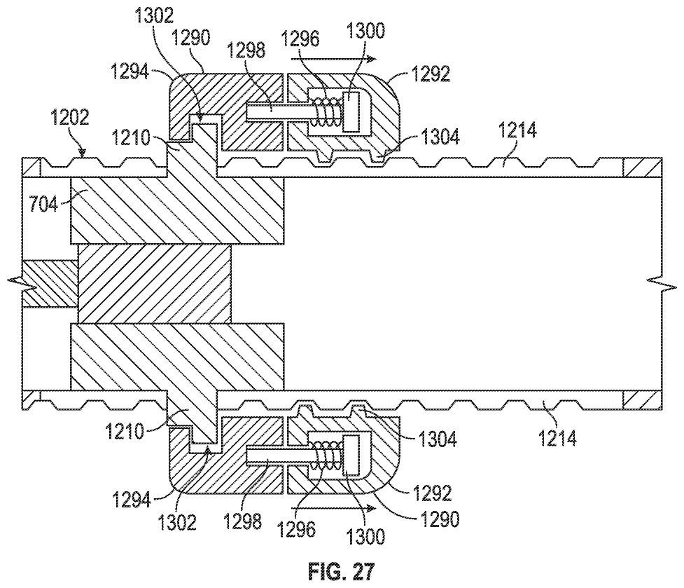

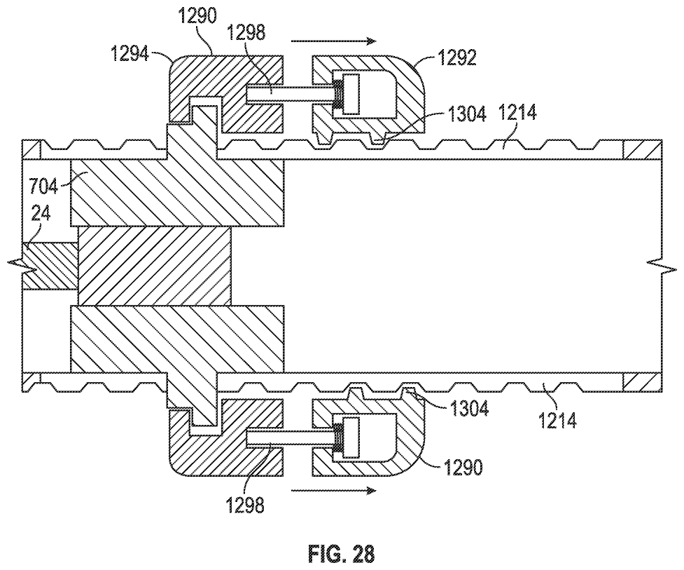

FIGS. 27-28 illustrate a cross sectional view along a mid line of an example in which the release knob 1290 is configured to split upon a tension force being applied to the manifold subassembly 24. The release knob 1290, for example, may include a first portion or proximal portion 1292 and a second portion or distal portion 1294. The release knob 1290 may split along a middle of the release knob 1290 between the proximal portion 1292 and the distal portion 1294, with a displacement body 1296 such as a spring drawing the distal portion 1294 towards the proximal portion 1292. The displacement body 1296 may be positioned on the actuator in the form of the release knob 1290. A connector 1298 such as a pin may connect the proximal portion 1292 with the distal portion 1294, and may include a piston head 1300 that may engage with the displacement body 1296.

The displacement body 1296 may be configured to allow the manifold subassembly to move proximally or distally relative to the control mechanism to automatically reduce the tension force or the compression force in the at least one of the one or more shafts.

The second portion or distal portion 1294 may include an engagement portion 1302 that may be configured to engage the engagement surfaces 1210 of the manifold adapter 704. The first portion or proximal portion 1292 may include threading 1304 that may be configured to engage the threading 1202 of the handle 14.

The release knob 1290 may split upon a tension force being applied to the manifold subassembly 24. The release knob 1290 may split in response to the release knob 1290 being rotated in a proximal direction, which may produce a tension in the manifold subassembly 24, or by a tension force otherwise being produced in the manifold subassembly 24 (e.g., via movement of other shafts of the elongate catheter 15 or other forces applied to the manifold subassembly 24). The release knob 1290 may split to automatically reduce a tension force in the shaft comprising the manifold subassembly 24.

FIG. 28 illustrates the separation of the proximal portion 1292 and the distal portion 1294 upon a tension force being applied to the manifold subassembly 24.

In examples, the separation of the proximal portion 1292 and the distal portion 1294 may result in the connector 1298 being exposed. The connector 1298, for example, may be uncovered by the proximal portion 1292 or distal portion 1294 and the exposed length of the connector 1298 may serve as a visual indicator of the tension force. The indicator may comprise the displacement of at least the body comprising the proximal portion 1292 of the actuator from the body comprising the distal portion 1294 of the actuator, and the displacement of the adapter 704 from the proximal portion 1292. The indicator may further comprise the displacement body 1296 in the form of the spring coupling the proximal portion 1292 to the distal portion 1294. The amount or length of the connector 1298 that is exposed may serve as the indicator of the tension force. In examples, a scaling or other form of marker may be positioned on the connector 1298 to indicate the amount of force. For example, a greater amount of the scaling may be shown due to a greater amount or length of the connector 1298 being exposed based on the force applied to the manifold subassembly 24.

In examples, the displacement body 1296 may be configured to allow the portions 1292, 1294 of the release knob 1290 to be drawn towards each other upon a compressive force being applied to the manifold adapter 704. A space between the portions 1292, 1294 of the displacement body 1296 may be reduced. As such, the release knob 1290 may allow for a reduction of a tension force and/or a compressive force with the manifold adapter 704.

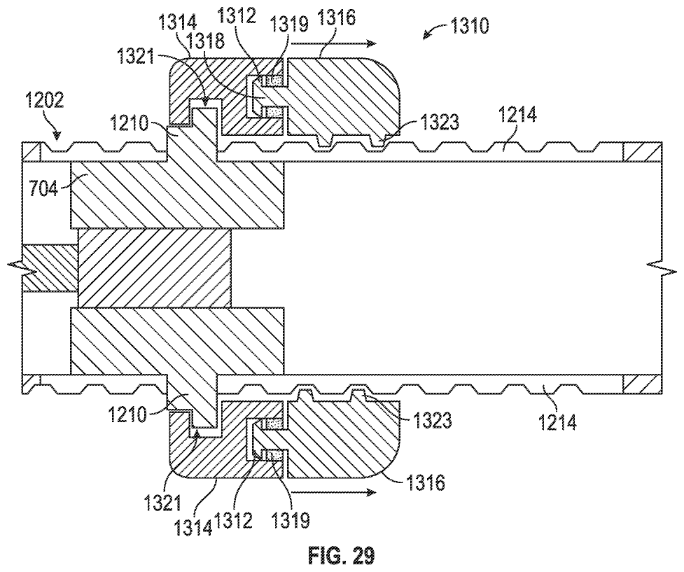

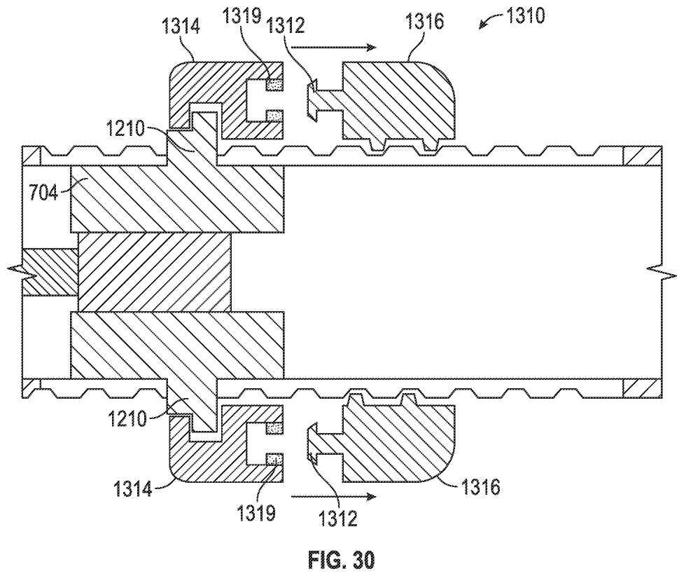

FIGS. 29-30 illustrate a cross sectional view along a mid line of an example in which the actuator in the form of the release knob 1310 is configured to disengage from the manifold adapter 704 with a releasable coupler 1312. The release knob 1310, for example, may include a proximal portion 1316 and a distal portion 1314 and the proximal portion 1316 may couple to the distal portion 1314 with the releasable coupler 1312. The proximal portion 1316 may include threading 1323 configured to engage the threading 1202 of the handle 14. The distal portion 1314 may include an engagement portion 1321 configured to engage the engagement surfaces 1210 of the manifold adapter 704. The distal portion 1314 may comprise a displacement body configured to allow the manifold subassembly to move proximally or distally relative to the control mechanism to automatically reduce the tension force or the compression force in the at least one of the one or more shafts.

The releasable coupler 1312 may comprise a pin with a wide end 1318, or may have another configuration. The releasable coupler 1312 may extend from the proximal portion 1316 of the release knob 1310 to the distal portion 1314 of the release knob 1310. The releasable coupler 1312 may engage a coupling body 1319 that may be positioned on the distal portion 1314. The coupling body 1319 may comprise a surface for the wide end 1318 of the releasable coupler 1312 to engage to retain the proximal portion 1316 of the release knob 1310 to the distal portion 1314 of the release knob 1310.

In examples, the coupling body 1319 may comprise a deformable body, such as an elastomeric body that may be configured to deform upon a sufficient force being applied to the releasable coupler 1312. The deformation of the coupling body 1319 may allow the releasable coupler 1312 to disengage from the distal portion 1314 and disengage the proximal portion 1316 from the distal portion 1314 of the release knob 1310. The coupling body 1319 may be configured such that a threshold force may deform the coupling body 1319 to allow the releasable coupler 1312 to disengage.

In examples, other configurations of releasable couplers 1312 may allow the releasable coupler 1312 to disengage, for example, spring arms or detents, or other configurations of releasable couplers 1312 may be utilized. The configuration of the releasable coupler 1312 show in FIG. 29 may be inverted in examples, with the releasable coupler 1312 extending from the distal portion 1314 to a coupling body 1319 positioned on the proximal portion 1316.

Upon a threshold tension in the manifold adapter 704 being met, the distal portion 1314 may release from the proximal portion 1316 by the releasable coupler 1312 being released. The distal portion 1314 may release in response to the release knob 1310 being rotated in a proximal direction, which may produce a tension in the manifold subassembly 24, or by a tension force otherwise being produced in the manifold subassembly 24 (e.g., via movement of other shafts of the elongate catheter 15 or other forces applied to the manifold subassembly 24). The release knob 1310 may split to automatically reduce a tension force in the shaft comprising the manifold subassembly 24.

Referring to FIG. 30, upon the releasable coupler 1312 being released, the distal portion 1314 may be configured to slide along with the adapter 704, to reduce the tension within the adapter 704. The adapter may disengage from the actuator to automatically reduce the tension force or compression force in at least one of the one or more shafts. The disengagement and separation of the portions of the actuator (the body comprising the distal portion 1314 from the body comprising the proximal portion 1316) may comprise a visual indicator of the tension force or the compression force in the at least one of the one or more shafts.

In the examples of FIGS. 24-30, the actuator of the control mechanism includes a first portion and a second portion that is configured to displace relative to the first portion to automatically reduce the tension force or the compression force in the at least one of the one or more shafts.

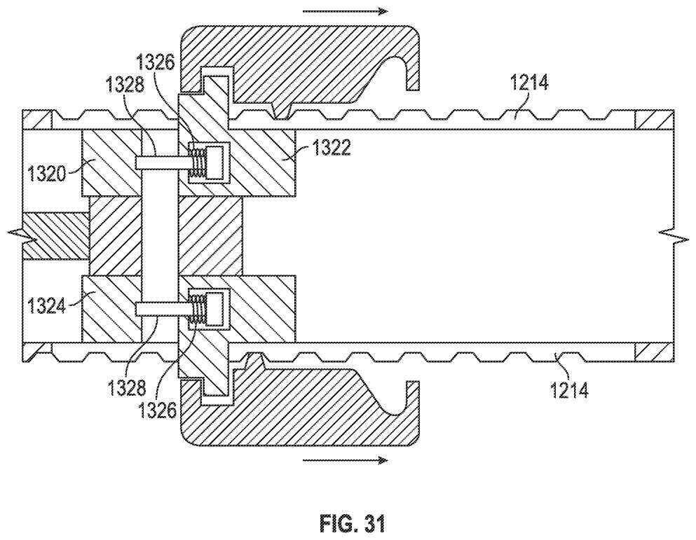

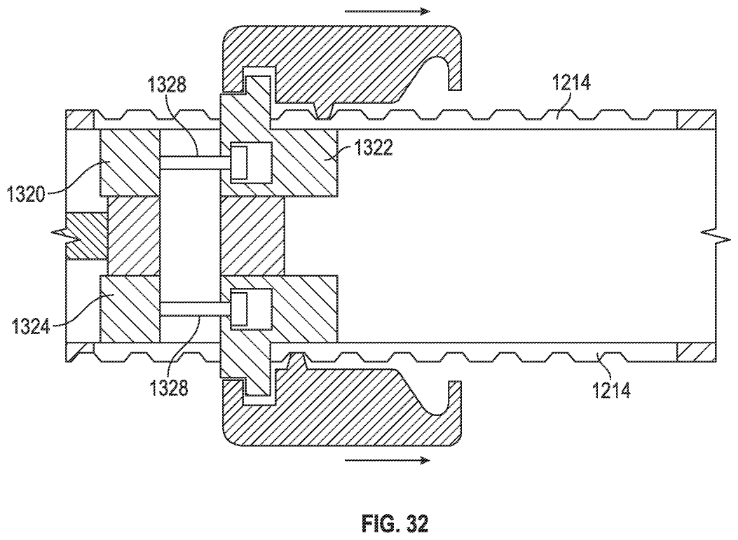

In examples, an adapter may be configured to have portions displace relative to each other to reduce tension or compression within a shaft coupled to the adapter. FIGS. 31-32, for example, illustrate an example in which the adapter 1320 includes a first portion or proximal portion 1322 and a second portion or distal portion 1324. Displacement bodies 1326 and connectors 1328 may be utilized in a similar manner as with the release knob disclosed herein to reduce a tension or compression within a shaft coupled to the adapter 1320. The displacement body 1326 may be configured to allow the manifold subassembly to move proximally or distally relative to the control mechanism to automatically reduce the tension force or the compression force in the at least one of the one or more shafts. The displacement body 1326 may be positioned on the adapter 1320.

The second portion or distal portion 1324 may be configured to displace relative to the first portion or proximal portion 1322 to reduce tension or compression within a shaft coupled to the second portion or distal portion 1324. The distal portion 1324 may move distally relative to the proximal portion 1322 to reduce a tension force in the shaft, and may move proximally relative to the proximal portion 1322 to reduce a compression force in the shaft. The connectors 1328 may allow the portions 1322, 1324 to remain engaged with each other upon the relative movement of the portions 1322, 1324. FIG. 32, for example, illustrates the distal portion 1324 displacing relative to the proximal portion 1322.

In examples, the second portion or distal portion 1324 of the adapter 1320 may be configured to disengage from the first portion or proximal portion 1322 of the adapter 1320 in a similar manner as discussed in regard to the examples of FIGS. 29 and 30, or in another manner.

In the examples of FIGS. 22-32, the force reduction mechanisms may be configured to allow at least one of the one or more shafts of the elongate catheter 15 or delivery device to move proximally or distally relative to the control mechanism to automatically reduce a tension force or a compression force in the at least one of the one or more shafts. The one or more shafts may be configured to displace relative to one or more of the actuator or the adapter to automatically reduce the tension force or compression force in the at least one of the one or more shafts.

The examples of FIGS. 22-32 may be utilized with a manifold shaft or manifold subassembly 24, or may be utilized with any other shaft or assembly or subassembly of a delivery system. For example, the examples of FIGS. 22-32 may be implemented with an outer sheath or outer sheath subassembly 20, or any other shaft or assembly or subassembly disclosed herein. The rail subassembly 21, mid-shaft subassembly 22, release subassembly 23, or nose cone assembly may utilize the examples of FIGS. 22-32. Any feature of a control mechanism, such as a control knob disclosed herein may be utilized in combination or utilized in alternative with the examples of FIGS. 22-32. Any feature of an adapter disclosed herein may be utilized in combination or utilized in alternative with the examples of FIGS. 22-32.

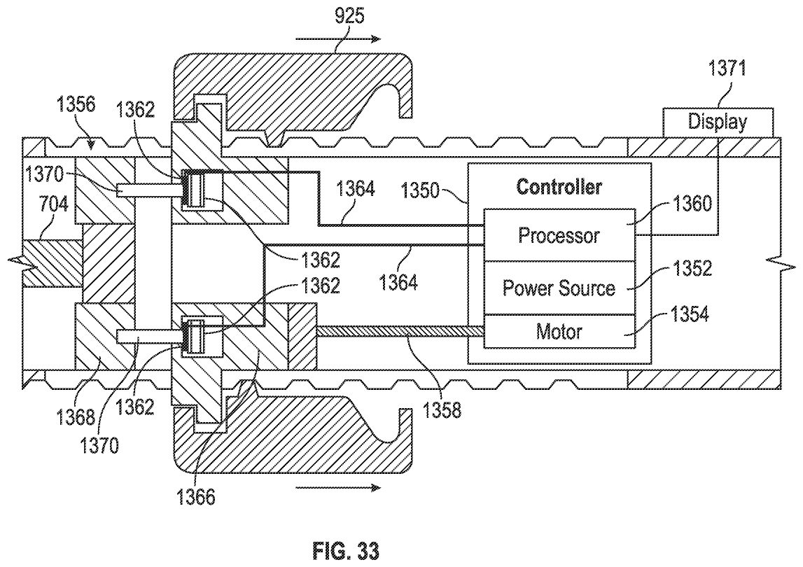

In examples, the force reduction mechanism may comprise an electric drive. The electric drive may be configured to automatically reduce a tension force or a compression force in at least one of the one or more shafts by moving at least one of the one or more shafts. FIG. 33, for example, illustrates an example in which an electric drive may comprise a controller 1350 that may include a motor 1354. The controller 1350 may comprise an electric device and may include a power source 1352 for powering components of the controller 1350 or other components of the system. The power source 1352, for example, may comprise a battery, a capacitor, or a power input (e.g., power plug) into the controller 1350, among other forms of power sources 1352.

The controller 1350 may include a motor 1354 that may be configured to apply a force to a portion of the system, such as an adapter 1356 of the system, or directly to a shaft of the system. The force applied by the motor 1354 may reduce a tension force or a compression force in at least one of the shafts of the system. The motor 1354, for example, may comprise a piston drive or screw drive 1358 or other form of motor configured to apply a force to a portion of the system. The motor 1354 may be powered by the power source 1352.

The controller 1350 may include a processor 1360. The processor 1360 may comprise a central processing unit (CPU) and may comprise a single CPU or multiple CPUs utilized in combination. The processor 1360 may be positioned within the handle or may be positioned remote, and may be utilized in an internet or cloud computing environment as desired. Other forms of processors may be utilized as desired. The processor 1360 may be powered by the power source 1352.

The processor 1360 may be configured to control the motor 1354. The processor 1360 may control the motor 1354 in response to sensor signals received by the processor 1360. Sensors 1362, for example, may be provided that may sense a tension or compression force applied to the adapter 1356 or otherwise present in a shaft coupled to the adapter 1356. The signals from the sensors 1362 may be transmitted via signal conduits 1364 to the processor 1360.

The adapter 1356, for example, may include a proximal portion 1366 and a distal portion 1368, with the proximal portion 1366 configured to move relative to the distal portion 1368 in response to a tension or compression force applied to the shaft coupled to the adapter 1356, similar to an example as shown in FIGS. 31-32. Connectors 1370, similar to connectors 1328 shown in FIGS. 31-32, may slide relative to the proximal portion 1366 and the sensors 1362 may sense the force applied to the connectors 1370 and accordingly the adapter 1356 and the shaft coupled to the adapter 1356.

The signals from the sensors 1362 may be transmitted via the signal conduits 1364 to the processor 1360. The processor 1360 may process the signals from the sensors 1362 to determine an amount of load on the adapter 1356 and the shaft coupled to the adapter 1356. The processor 1360 may provide a variety of functions in response.

In examples, the processor 1360 may operate the motor 1354 to cause the motor 1354 to move the adapter 1356 to reduce the tension or compression force in the adapter 1356. The motor 1354 may control the adapter 1356 upon an increased tension or compression force being sensed by the sensors 1362. The processor 1360 and motor 1354 may operate automatically to reduce the tension or compression force.

The motor 1354 may apply a force to a proximal portion 1366 of the adapter 1356 as shown in FIG. 33, or may apply a force to a distal portion 1368 of the adapter 1356 or directly to the shaft coupled to the adapter 1356 in examples. The motor 1354 in examples may apply a force to an actuator of a control mechanism such as a release knob 925 or other actuator as desired.

In the example shown in FIG. 33, the processor 1360 and motor 1354 may serve as an override for a manual control provided by a user. For example, a user may operate an actuator such as the release knob 925 manually. If an increased tension or compression force is detected via the sensors 1362, the processor 1360 may operate the motor 1354 to override the manual control and reduce the tension or compression in the adapter 1356 and the shaft coupled to the adapter 1356. The processor 1360 may operate based on feedback signals from the sensors 1362, which may be real time feedback to the processor 1360 from the sensors 1362. The controller 1350 may automatically move the shaft coupled to the adapter 1356 distally to reduce the tension force or proximally to reduce the compression force.

In examples, a memory may be provided that may store a threshold amount of tension or compression force for the processor 1360. The processor 1360 may determine if a threshold amount of tension or compression force is met based on the signals from the sensors 1362 and operate the motor 1354 upon the threshold being met. The memory may be programmed with threshold values that may be varied by a user, or may include preset threshold values.

In examples, the processor 1360 may provide a signal to a display 1371 of the amount of force sensed by the sensors 1362. A user may be able to view the display 1371 as a visual indicator to determine the amount of force sensed by the sensors 1362 and applied to the adapter 1356. Other forms of indicators, such as auditory or haptic indicators may be utilized. The indicators may comprise electric indicators. For example, an electric speaker may be provided to make sound as an auditory indicator. A vibrating motor (e.g., an eccentric motor), may be utilized to comprise a haptic indicator, among other forms of indicators that may be utilized.

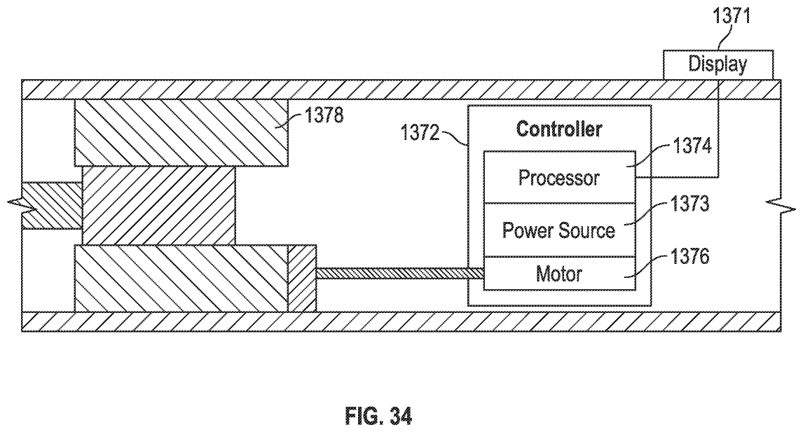

FIG. 34 illustrates an example in which a controller 1372 may operate automatically, without a user manually operating a release knob 925 as shown in FIG. 33. The processor 1374 may control the motor 1376 to move the adapter 1378 and the shaft coupled to the adapter 1378. A power source 1373 may be provided that may be similar to the power source 1352. The controller 1372 may be configured to operate according to a programmed operation profile, or may receive controls from a control device operated by a user (which may be controls on the handle or controls provided remotely).

Upon a threshold tension or compression force being sensed in the motor 1376, the processor 1374 may operate to automatically reduce the tension or compression within the adapter 1378 and the shaft coupled to the adapter 1378. For example, a greater than threshold force experienced by the motor 1376 may indicate a compression or tension force in the adapter 1378. The motor 1376 may operate to reduce the compression or tension force. The controller 1372 may automatically move the shaft coupled to the adapter 1378 distally to reduce the tension force or proximally to reduce the compression force.

In examples, the controller 1372 may operate in response to feedback signals received from one or more sensors.

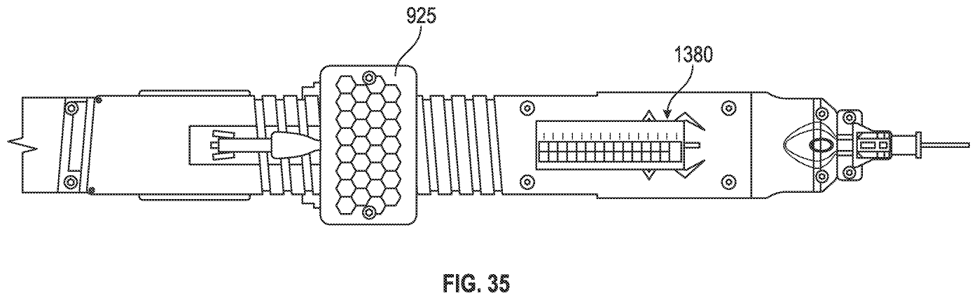

FIG. 35 illustrates a side view of a display 1380 that may be utilized as a visual indicator of the tension or compression force in a shaft of the system. The shaft, for example, may comprise the manifold subassembly 24 or another shaft of the system. The display 1380 may comprise an electric display of the amount of tension or compressive force experienced by the shaft. An electric scaling may be provided on the display 1380 (e.g., an amount of the display that is illuminated or otherwise shown to indicate the force), or a numerical value, or other form of visual indicator may be utilized. An electric scaling, for example, may comprise a colored scale or series of graduated light (e.g., light emitting diodes, or other light) that indicate a safe zone or a harm zone for the respective shaft. A user may be able to determine the tension or compression based on the display 1380 to reduce the tension or compression by operating an actuator such as the release knob 925. The display 1380 may be operated by a controller in a similar manner as the display 1371 discussed in regard to FIGS. 33 and 34.

Examples herein may utilize an indicator such as a visual, auditory, or tactile indicator. Although examples are discussed in regard to the manifold subassembly, other shafts or subassemblies may utilize the force reduction mechanisms disclosed herein. For example, the examples of FIGS. 33-35 may be utilized with a manifold shaft or manifold subassembly 24, or may be utilized with any other shaft or assembly or subassembly of a delivery system. For example, the examples of FIGS. 33-35 may be implemented with an outer sheath or outer sheath subassembly 20, or any other shaft or assembly or subassembly disclosed herein. The rail subassembly 21, mid-shaft subassembly 22, release subassembly 23, or nose cone assembly may utilize the examples of FIGS. 33-35. Any feature of a control mechanism, such as a control knob disclosed herein may be utilized in combination or utilized in alternative with the examples of FIGS. 33-35. Any feature of an adapter disclosed herein may be utilized in combination or utilized in alternative with the examples of FIGS. 33-35.

In the examples of FIGS. 16-35, the force reduction mechanisms may be configured to automatically reduce the tension force or the compression force in the at least one of the one or more shafts upon a respective threshold tension force or threshold compression force being met.

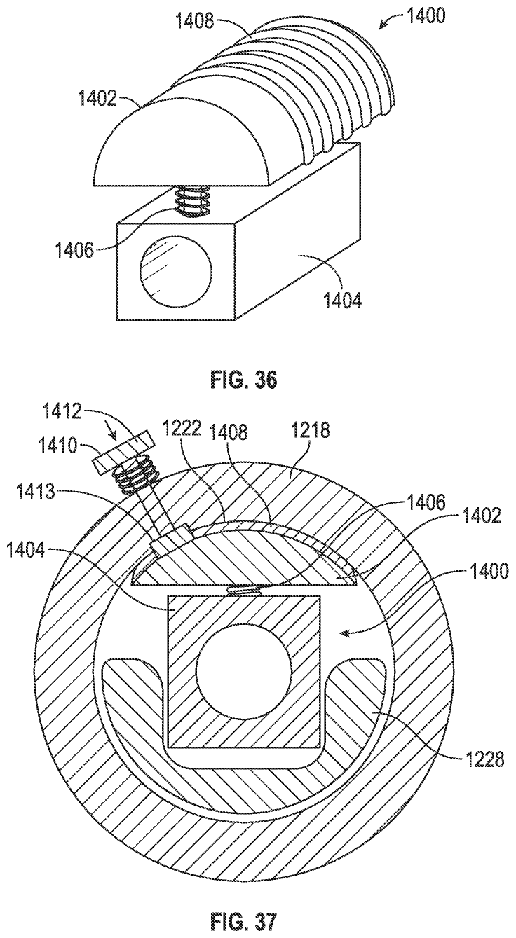

In examples, the force reduction mechanisms may be user actuated. FIG. 36, for example, illustrates an example of an adapter 1400 that may be user actuated to reduce a tension or compression of a shaft coupled to the adapter 1400. The adapter 1400 may include an engagement body 1402 and a slide body 1404 coupled to the engagement body 1402.

The engagement body 1402 may be coupled to the slide body 1404 with a displacement body 1406 in the form of a spring. The displacement body 1406 may be positioned on the adapter 1400. The displacement body 1406 may press the engagement body 1402 of the adapter 1400 towards an actuator and may be configured to be moved to disengage the adapter 1400 from the actuator. The displacement body 1406 may be biased to move the engagement body 1402 away from the slide body 1404, and the bias may be overcome with a pressing of the engagement body 1402 towards the slide body 1404. The displacement body 1406 may be configured to allow the shaft coupled to the adapter 1400 to move proximally or distally relative to the control mechanism to automatically reduce the tension force or the compression force in the at least one of the one or more shafts.

The engagement body 1402 may include threading 1408 that may be configured to engage threading 1222 of the rotating body 1218 shown in FIG. 18. Upon the engagement body 1402 being pressed towards the slide body 1404, the threading 1408 may disengage from the threading 1222, allowing the slide body 1404 to slide within the interior lumen 1220 shown in FIG. 18.

FIG. 37, for example, illustrates a cross sectional view of the adapter 1400 within the interior lumen 1220 shown in FIG. 18. The engagement body 1402 is shown engaged with the threading 1222 of the rotating body 1218.

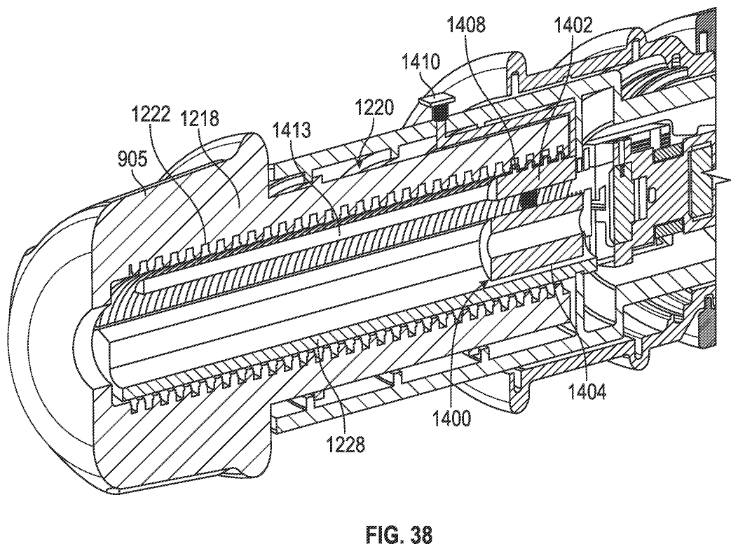

In examples, an actuator 1410 may be utilized to press against the engagement body 1402 and press the engagement body 1402 towards the slide body 1404 to disengage the threading 1408 from the threading 1222 of the rotating body 1218. The actuator 1410, for example, may comprise a pressing body 1413 that presses against the engagement body 1402. The pressing body 1413 as shown in FIG. 38, for example, may comprise an elongate bar positioned within the interior lumen 1220. The elongate bar may be configured to press against the adapter 1400 at a variety of longitudinal positions of the adapter 1400. The actuator 1410 may be configured for a user to operate to activate the force reduction mechanism.

A button 1412 or other form of actuator may be accessible by a user to press the pressing body 1413 against the engagement body 1402. The displacement body 1406 may resist the force applied by the pressing body 1413.

Referring to FIG. 38, in operation a user may control an actuator such as the capsule knob 905 to slide the adapter 1400 and advance or retract the outer sheath subassembly 20. After performing a desired operation, the user may press the actuator 1410 to move the pressing body 1413 downward. The pressing body 1413 may depress the engagement body 1402 and release the threading 1408 of the engagement body 1402 from the threading 1222 of the rotating body 1218. The adapter 1400 may be free to slide independent of the capsule knob 905, and may thus automatically reduce any tension or compression force within the outer sheath subassembly 20.

The user may release the actuator 1410 to cause the engagement body 1402 to reengage with the rotating body 1218.

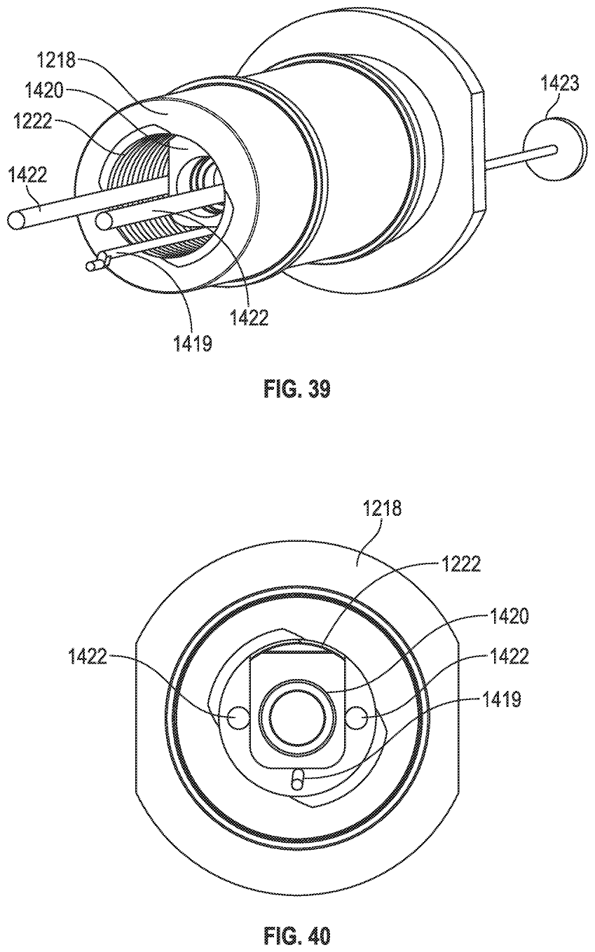

FIGS. 39-42 illustrate an example in which a cam 1419 or other form of actuator may be utilized to engage or disengage an adapter 1420 from the threading 1222 shown in FIG. 18 for example. The cam 1419 accordingly may engage or disengage the adapter 1420 from the actuator (such as the capsule knob 905). The cam 1419 may be configured for a user to operate to activate the force reduction mechanism. The cam 1419 may comprise a displacement body that may be configured to allow the shaft coupled to the adapter 1420 to move proximally or distally relative to the control mechanism to automatically reduce the tension force or the compression force in the at least one of the one or more shafts.

The cam 1419 may comprise a cam shaft that may extend longitudinally along the length of the interior lumen 1220 shown in FIG. 18 for example. The cam 1419 may replace the bottom surface of the beam 1228 shown in FIG. 18, with rods 1422 extending along the sides of the adapter 1420.

The cam 1419 may be user actuated, with a control device 1423 present to allow the cam 1419 to be rotated as desired. The control device 1423 may be configured to be operated by a user and may comprise a knob, or a button, or other form of control device 1423. In a raised position as show in FIGS. 39 and 40, the cam 1419 may press the adapter 1420 against the threading 1222. The rotation of the rotating body 1218 may cause the adapter 1420 and the outer sheath subassembly 20 to slide.

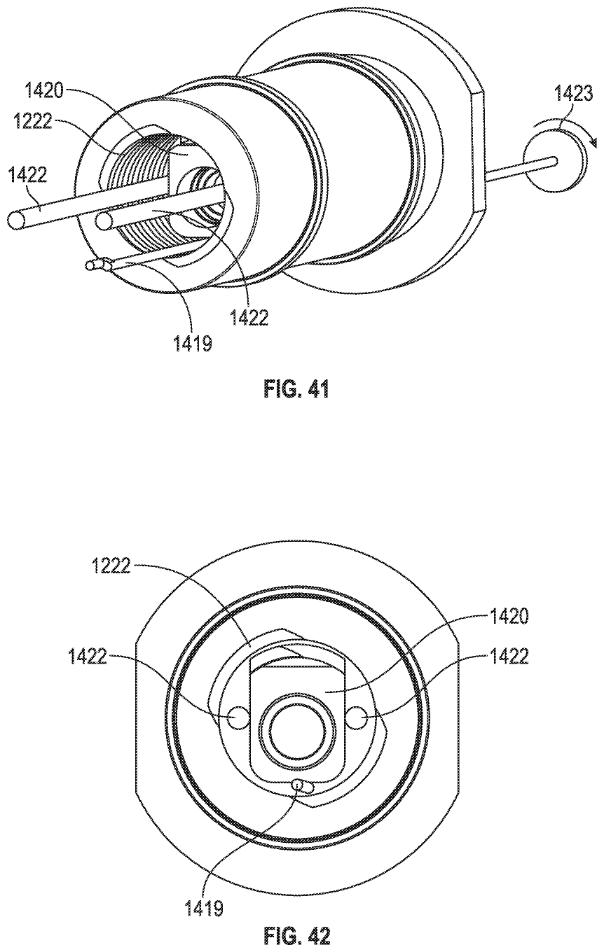

At a desired time, the user may rotate the cam 1419 as shown in FIGS. 41 and 42. The user, for example, may rotate the control device 1423 to lower the cam 1419. The adapter 1420 may disengage from the threading 1222 and may be able to slide along the rods 1422 to automatically reduce any tension or compression force within the outer sheath subassembly 20.

The cam 1419 may be rotated back into the position shown in FIGS. 39 and 40 to reengage the threading 1222 at a desired time.

Force reduction mechanisms disclosed herein may include clutches, mechanical push buttons, electronic drives, and/or magnets.

In the examples of FIGS. 36-42, the force reduction mechanisms may be configured to allow at least one of the one or more shafts of the elongate catheter 15 or delivery device to move proximally or distally relative to the control mechanism to automatically reduce a tension force or a compression force in the at least one of the one or more shafts. The one or more shafts is configured to displace relative to the actuator to automatically reduce the tension force or compression force in the at least one of the one or more shafts. The adapter may disengage from the actuator to automatically reduce the tension force or compression force in at least one of the one or more shafts.

Any example of subassembly or shaft disclosed herein may utilize a force reduction mechanism that is user actuated. For example, the examples of FIGS. 36-42 may be utilized with an outer sheath or outer sheath subassembly 20, or may be utilized with any other shaft or assembly or subassembly of a delivery system. For example, the examples of FIGS. 36-42 may be implemented with a manifold shaft or manifold subassembly 24, or any other shaft or assembly or subassembly disclosed herein. The rail subassembly 21, mid-shaft subassembly 22, release subassembly 23, or nose cone assembly may utilize the examples of FIGS. 36-42. Any feature of a control mechanism, such as a control knob disclosed herein may be utilized in combination or utilized in alternative with the examples of FIGS. 36-42. Any feature of an adapter disclosed herein may be utilized in combination or utilized in alternative with the examples of FIGS. 36-42.

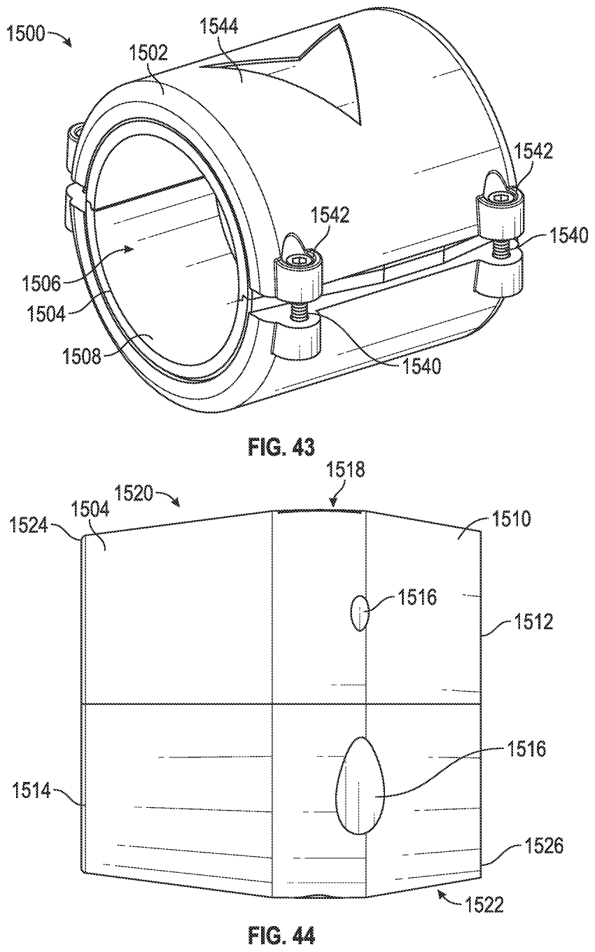

FIGS. 43-59 illustrate implementations including a control mechanism for moving one or more shafts of an elongate catheter or delivery device. The control mechanism may include an actuator knob having a first portion and a second portion that is configured to rotate relative to the first portion to automatically limit a tension force or a compression force in at least one of the shafts transmitted by the actuator knob to the at least one of the shafts. The rotation may prevent a tension force or compression force in the at least one of the shafts from exceeding a threshold force. For example, the rotation may prevent a user from exceeding a respective threshold tension force or threshold compression force in at least one of the shafts.

The rotation may prevent the user from over tensioning or over compressing at least one of the shafts by applying an undue force (e.g., torque) upon the actuator knob. A reduced possibility of damage to the shaft from the over tensioning or over compression may result.

Referring to FIG. 43, for example, a perspective view of an actuator knob 1500 of a control mechanism is shown. The actuator knob 1500 may comprise a control knob of the delivery system, which may comprise a release knob as disclosed herein or another form of knob of the delivery system as desired. In examples, other forms of actuators may be utilized as desired.

The actuator knob 1500 may include a first portion or inner portion 1504 and a second portion or outer portion 1502. The outer portion 1502 may comprise an outer body extending around the inner portion 1504. The outer portion 1502 may comprise an outer shell extending around the inner portion 1504. The outer portion 1502 may surround the inner portion 1504 or may have another configuration. The outer portion 1502 may be configured to displace relative to the inner portion 1504. The displacement may comprise a rotational displacement. The displacement may automatically limit the tension force or compression force in at least one of the shafts.

The inner portion 1504 may comprise an inner body that may be positioned within the outer portion 1502. The inner portion 1504 may comprise a sleeve configured to extend over an outer surface of a handle 14 as shown in FIG. 14, for example. The inner portion 1504 may include a central channel 1506 that the handle 14 may be positioned within. The inner portion 1504 may have an inner surface 1508 that may face towards the central channel 1506 and may have an outer surface 1510 (marked in FIG. 44) that may face opposite the inner surface 1508.

FIG. 44 illustrates a side view of the inner portion 1504. The inner portion 1504 may include multiple parts, including an upper shell 1512 and a lower shell 1514 that may be joined together via couplers (e.g., screws or snaps) extending through coupler channels 1516 (e.g., screw holes) in the outer surface 1510 of the inner portion 1504.

As shown in FIG. 44, the outer surface 1510 of the inner portion 1504 may have a varied diameter. For example, a central portion 1518 of the inner portion 1504 may have a greater outer diameter than a distal portion 1520 and a proximal portion 1522 of the inner portion 1504. The outer surface 1510 may have a tapered shape. The distal portion 1520 may taper radially inward from the central portion 1518 in a direction towards the distal end 1524 of the inner portion 1504. The proximal portion 1522 may taper radially inward from the central portion 1518 in a direction towards the proximal end 1526 of the inner portion 1504.

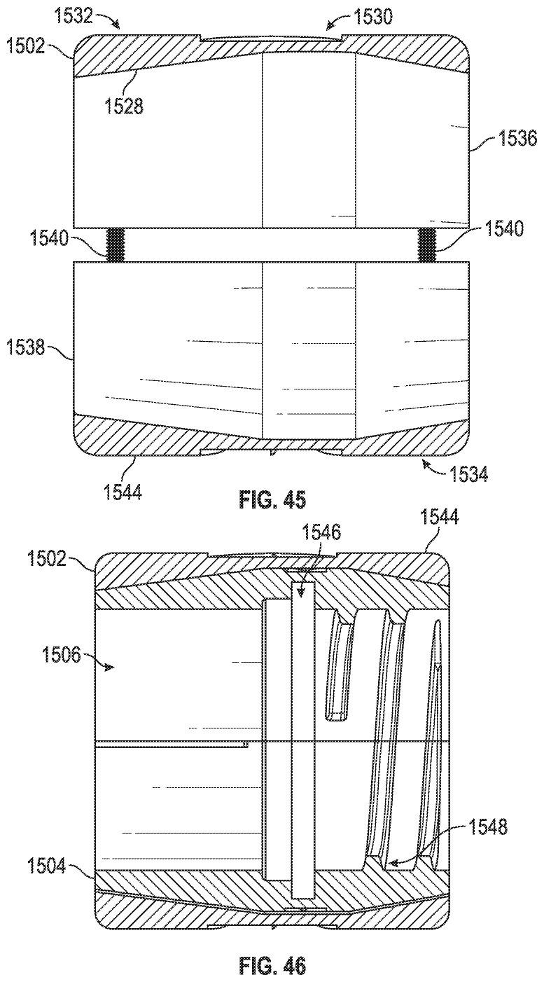

The configuration of the outer surface 1510 of the inner portion 1504 having a varied outer diameter may assist to reduce longitudinal or axial movement of the outer portion 1502 relative to the inner portion 1504. For example, the outer portion 1502 may have an inner surface 1528 (marked in FIG. 45) that conforms to a shape of the outer surface 1510 of the inner portion 1504. The outer portion 1502, for example, may have a central portion 1530 having a greater interior diameter than a distal portion 1532 and a proximal portion 1534 of the outer portion 1502. The shape of the inner surface 1528 may conform to the shape of the outer surface 1510 of the inner portion 1504 such that longitudinal or axial movement of the outer portion 1502 may be resisted by the greater diameter at the respective central portions 1518, 1530.

Referring to FIG. 45, a cross sectional view of the outer portion 1502 is shown. The outer portion 1502 may include multiple parts, including an upper shell 1536 and a lower shell 1538 that may be joined together via couplers 1540 (e.g., screws) extending through coupler channels 1542 (e.g., screw holes) of the outer portion 1502 (shown in FIG. 43).

The outer portion 1502 may have an outer surface 1544 that may face opposite the inner surface 1528. The outer surface 1544 may comprise a portion of the actuator knob 1500 for a user to grip, to apply a rotational force (i.e., a torque) upon the actuator knob 1500.

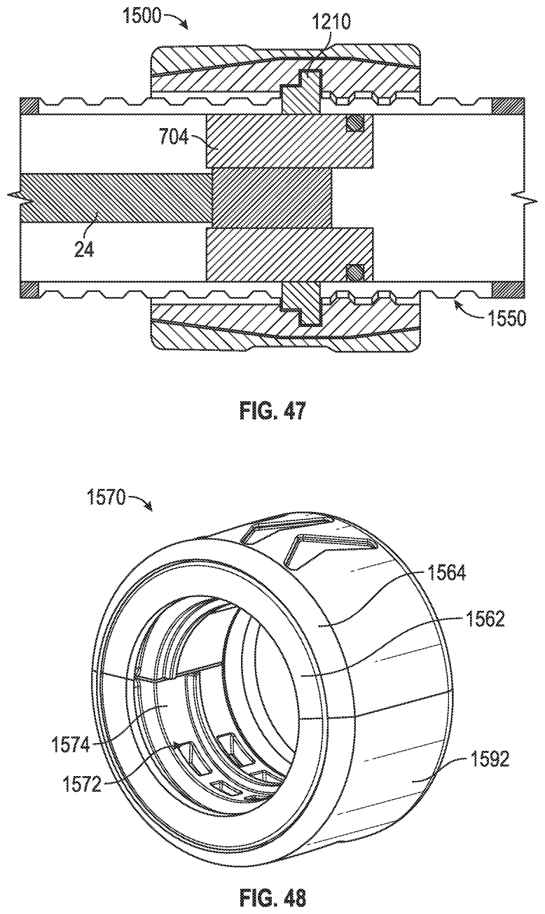

FIG. 46 illustrates a cross sectional view of the outer portion 1502 positioned upon the inner portion 1504. The inner portion 1504 may include an engagement portion 1546 that may be configured to receive engagement surfaces 1210 (marked in FIG. 47) of an adapter such as the manifold adapter 704. The inner portion 1504 may be configured to engage the adapter 704 coupled to the proximal end portion of one of the shafts. The inner portion 1504 may be configured to rotate about the adapter 704 and convey rotational motion of the actuator knob 1500 to a longitudinal or axial motion of the adapter 704. The engagement portion 1546 may be configured to convey rotational motion of the actuator knob 1500 into a longitudinal or axial motion of the adapter 704 in a similar manner as discussed regarding other actuators disclosed herein. The inner portion 1504 may further include threading 1548 that may engage threading 1550 (marked in FIG. 47) on the handle, to cause rotational movement of the actuator knob 1500 about the handle to produce a longitudinal or axial movement of the actuator knob 1500 relative to the handle. FIG. 47, for example, illustrates an exemplary configuration of the actuator knob 1500 upon a handle. A proximal movement of the actuator knob 1500 may result in a proximal movement of the adapter 704, and a resulting proximal movement or tension force in the shaft 24. A distal movement of the actuator knob 1500 may result in a distal movement of the adapter 704, and a resulting distal movement or compression force in the shaft 24.

The actuator knob 1500 may include a bearing surface, which may comprise one or more of the outer surface 1510 of the inner portion 1504 or the inner surface 1528 of the outer portion 1502. The bearing surface may allow the outer portion 1502 to rotate relative to the inner portion 1504. The bearing surface may comprise a friction surface that may produce friction between the outer surface 1510 of the inner portion 1504 and the inner surface 1528 of the outer portion 1502. The bearing surface may have a varied diameter in a similar manner as discussed regarding the respective surfaces 1510, 1528.

The friction may allow the inner portion 1504 to rotate with the outer portion 1502 and maintain a rotational position of the inner portion 1504 relative to the outer portion 1502. Such a configuration may allow the outer portion 1502 to convey rotational motion to inner portion 1504 and correspondingly convey the rotational motion to longitudinal or axial motion of the adapter 704.

However, upon a threshold tension or compression force in the shaft 24 being met, longitudinal movement of the inner portion 1504 may be resisted, which may result in a resistance to rotational movement of the inner portion 1504. The threshold tension or compression force in the shaft 24 may be met in a variety of manners as disclosed herein. For example, a force upon the shaft 24 by another shaft of the delivery system, or due to an implant deployment procedure may produce the threshold tension or compression force. The threshold tension or compression force may be provided due to friction between shafts of the delivery system in examples, or a force of deployment or recapture of an implant, among other reasons. The shaft may be impeded from longitudinal or axial motion, which may cause the threshold tension or compression force to be met by the force of a user upon the actuator knob 1500.

Upon the threshold tension or compression force being met, the friction between the outer surface 1510 of the inner portion 1504 and the inner surface 1528 of the outer portion 1502 may be overcome and the outer portion 1502 may rotate relative to the inner portion 1504. The surfaces 1510, 1528, for example, may slide relative to each other, preventing the outer portion 1502 from transferring further rotational motion to the inner portion 1504. Longitudinal or axial movement of inner portion 1504 and the adapter 704, and correspondingly the shaft 24, may be reduced. Upon the tension or compression force in the shaft 24 being reduced, the surfaces 1510, 1528 may reengage with each other thus allowing the outer portion 1502 to convey a rotational motion to the inner portion 1504.

In examples, the threshold tension force or threshold compression force that may cause the outer portion 1502 to rotate relative to the inner portion 1504 may be adjustable. For example, referring to FIG. 43, a compression force of the outer portion 1502 upon the inner portion 1504 may be adjusted. The compression applied by the couplers 1540, for example, may be adjusted (e.g., by tightening or loosening screws, or through other methods). The outer portion 1502 may comprise an outer shell having an upper shell 1512 and a lower shell 1514. The compression of the outer shell upon the inner portion 1504 may be adjusted by adjusting the tightness of the couplers 1540. A greater compression of the outer portion 1502 upon the inner portion 1504 may increase the friction between the surfaces 1510, 1528 and may thus increase the threshold tension force or threshold compression force that may cause the outer portion 1502 to rotate relative to the inner portion 1504. A lesser compression of the outer portion 1502 upon the inner portion 1504 may decrease the friction between the surfaces 1510, 1528 and may thus decrease the threshold tension force or threshold compression force that may cause the outer portion 1502 to rotate relative to the inner portion 1504. As such, a user may set a threshold tension force or threshold compression force that may cause the outer portion 1502 to rotate relative to the inner portion 1504.

Various other configurations may be utilized in examples.

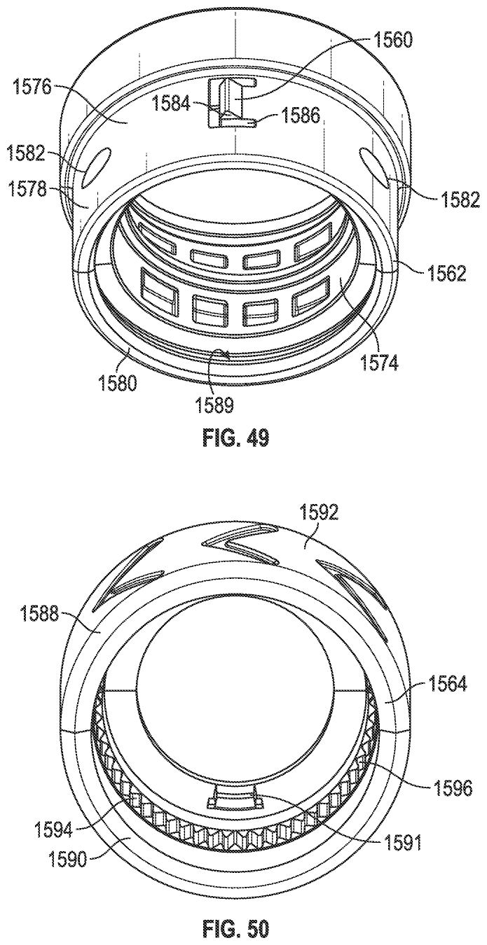

For example, FIGS. 48-52 illustrate an example having a displacement body 1560 configured to retain a rotational position of a first portion or inner portion 1562 relative to a second portion or outer portion 1564. The displacement body 1560 may be configured to release to allow the outer portion 1564 to rotate relative to the inner portion 1562. The displacement body 1560, for example, is marked in FIG. 49.

Referring to FIG. 48, a perspective view of an actuator knob 1570 of a control mechanism is shown. The actuator knob 1570 may comprise a control knob of the delivery system, which may comprise a release knob as disclosed herein or another form of knob of the delivery system as desired. In examples, other forms of actuators may be utilized as desired.

The actuator knob 1570 may include the inner portion 1562 and the outer portion 1564. The outer portion 1564 may comprise an outer body extending around the inner portion 1562. The outer portion 1564 may comprise an outer shell extending around the inner portion 1562. The outer portion 1564 may surround the inner portion 1562 or may have another configuration. The outer portion 1564 may be configured to displace relative to the inner portion 1562. The displacement may comprise a rotational displacement. The displacement may automatically limit the tension force or compression force in at least one of the shafts.

The inner portion 1562 may comprise an inner body that may be positioned within the outer portion 1564. The inner portion 1562 may comprise a sleeve configured to extend over an outer surface of a handle 14 as shown in FIG. 14, for example. The inner portion 1562 may include a central channel 1572 that the handle 14 may be positioned within. The inner portion 1562 may have an inner surface 1574 that may face towards the central channel 1572 and may have an outer surface 1576 (marked in FIG. 49) that may face opposite the inner surface 1574.

FIG. 49 illustrates a perspective view of the inner portion 1562 separate from the outer portion 1564. The inner portion 1562 may include multiple parts, including an upper shell 1578 and a lower shell 1580 that may be joined together via couplers (e.g., screws) extending through coupler channels 1582 (e.g., screw holes) in the outer surface 1576 of the inner portion 1562.

The displacement body 1560 may protrude from the outer surface 1576 of the inner portion 1562. The displacement body 1560, for example, may extend radially outward from the inner portion 1562. The displacement body 1560 may comprise a detent configured to retain a rotational position of the inner portion 1562 relative to the outer portion 1564. The displacement body 1560 may be configured to engage the outer portion 1564 with the inner portion 1562 to retain a rotational position of the outer portion 1564 relative to the inner portion 1562. The detent may comprise a protrusion 1584 configured to engage a surface of the outer portion 1564. The protrusion 1584 may comprise a tab positioned at an end of a lever arm 1586. The lever arm 1586 may be configured to deflect radially inward and disengage from the outer portion 1564 upon a force between the protrusion 1584 and the outer portion 1564 being exceeded.

In examples, the inner portion 1562 may include a coupling surface 1589 that may be configured to engage a coupler 1591 (marked in FIG. 50) of the outer portion 1564. The coupler 1591 may comprise a rotational coupler 1591 configured to allow for rotation of the outer portion 1564 relative to the inner portion 1562. The coupler 1591, for example, may comprise a hook configured to hook over the coupling surface 1589 of the inner portion 1562 and allow for rotation of the coupling surface 1589 relative to the coupler 1591.

FIG. 50 illustrates a perspective view of the outer portion 1564 separate from the inner portion 1562. The outer portion 1564 may include multiple parts, including an upper shell 1588 and a lower shell 1590 that may be joined together via couplers (e.g., screws). The couplers, for example, may extend through coupler channels (e.g., screw holes) of the outer portion 1564.

The outer portion 1564 may have an outer surface 1592. The outer surface 1592 may comprise a portion of the actuator knob 1570 for a user to grip, to apply a rotational force (i.e., a torque) upon the actuator knob 1570.

The outer portion 1564 may include an inner surface 1594 that may face opposite the outer surface 1592. The inner surface 1594 may face towards the outer surface 1576 of the inner portion 1562. The inner surface 1594 may include an engagement surface 1596 that the displacement body 1560 may be configured to engage. The engagement surface 1596, for example, may comprise a ridged surface for the displacement body 1560 to engage.

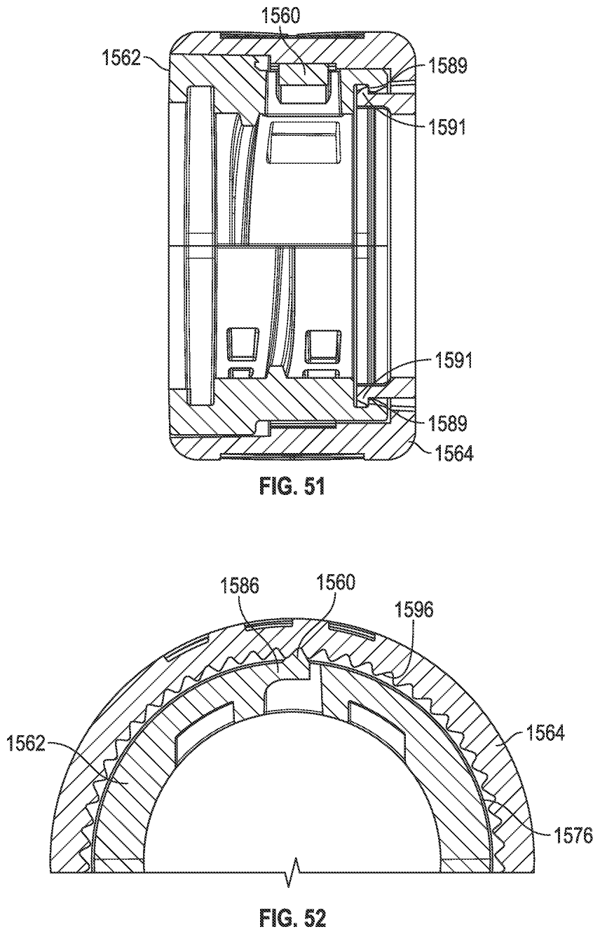

FIG. 51 illustrates a cross sectional view of the outer portion 1564 positioned upon the inner portion 1562. The engagement of the coupler 1591 with the coupling surface 1589 is shown. In examples, multiple couplers 1591 may be provided. For example, FIG. 51 illustrates at least two couplers 1591 being utilized. A greater or lesser number of couplers may be utilized as desired.

FIG. 52 illustrates a cross sectional view of the outer portion 1564 positioned upon the inner portion 1562 at a view perpendicular to the view shown in FIG. 51. The engagement of the displacement body 1560 with the engagement surface 1596 is shown. The displacement body 1560 extends radially towards the engagement surface 1596. The displacement body 1560 may be engaged with the engagement surface 1596 due to the displacement body 1560 being biased towards the engagement surface 1596. For example, the lever arm 1586 may be biased to press the displacement body 1560 against the engagement surface 1596.

The displacement body 1560 may release from the engagement surface 1596 upon a threshold tension or compression force in a shaft (e.g., shaft 24) being produced. Longitudinal or axial movement of the inner portion 1562 may be resisted, which may result in a resistance to rotational movement of the inner portion 1562. As such, the force of the displacement body 1560 against the engagement surface 1596 may be overcome and the outer portion 1564 may rotate relative to the inner portion 1562. The displacement body 1560 may release from the engagement surface 1596 to allow the surfaces 1576, 1594 to slide relative to each other. As such, the outer portion 1564 may be prevented from transferring further rotational movement to the inner portion 1562. Longitudinal movement of inner portion 1562 and an adapter (e.g., adapter 704), and correspondingly a shaft may be reduced. Upon the tension or compression force in the shaft being reduced, the displacement body 1560 may reengage with the engagement surface 1596, thus allowing the outer portion 1564 to convey a rotational motion to the inner portion 1562.

In examples, the number of displacement bodies 1560 and the orientation of the displacement bodies 1560 may be varied. For example, a plurality of the displacement bodies 1560 may be utilized in examples. In examples, the displacement bodies 1560 may be configured to protrude radially inward (e.g., with the engagement surface 1596 upon the inner portion 1562 and the one or more displacement bodies 1560 on the outer portion 1564 and extending radially inward towards the inner portion 1562). Other configurations may be utilized.

In examples, the threshold tension force or threshold compression force that may cause the outer portion 1564 to rotate relative to the inner portion 1562 may be adjustable. The engagement force of the displacement body 1560 with the outer portion 1564 and inner portion 1562 may be adjusted. For example, referring to FIG. 52, a configuration of one or more of the displacement body 1560 or the engagement surface 1596 may be adjusted. A size or shape of ridges of the engagement surface 1596, for example, may be varied to adjust the force required to be overcome to allow for rotational movement of the portions 1562, 1564 relative to each other. A number of the ridges may be varied as desired. In examples, the size, shape, or number of the displacement bodies 1560 may be adjusted. For example, an angle of the protrusion may be varied. A rigidity of the lever arm 1586 may be varied. Multiple displacement bodies 1560 may be positioned in a variety of locations as desired.

A configuration of the displacement body 1560 may be varied in examples.

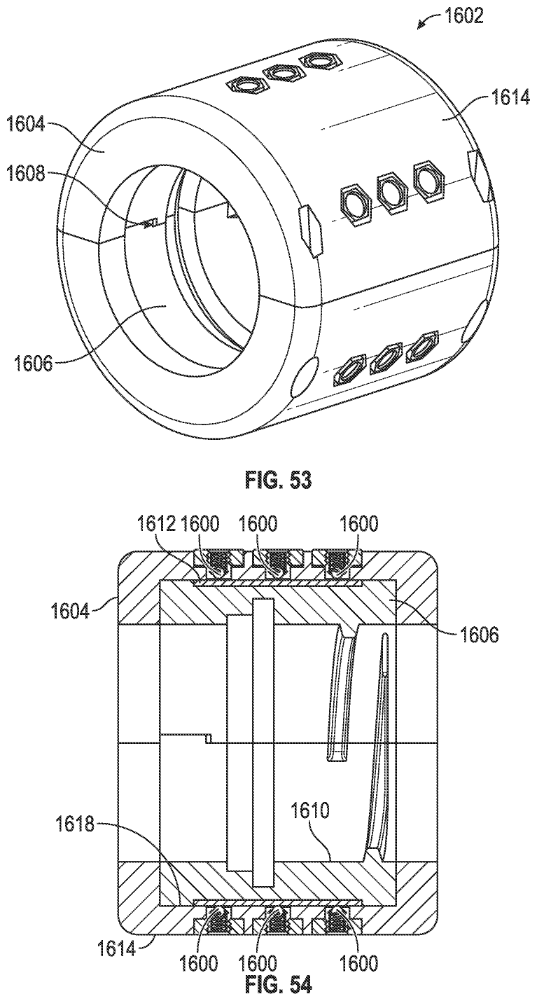

FIGS. 53-56, for example, illustrate an implementation in which a plurality of displacement bodies 1600 may be provided (marked in FIG. 54). The plurality of displacement bodies 1600 may be spaced from each other circumferentially and longitudinally. For example, a plurality of rows of the displacement bodies 1600 may be provided. The rows may comprise a plurality of the displacement bodies 1600 aligned longitudinally with each other. Each row may be spaced circumferentially from an adjacent row.

FIG. 53 illustrates a perspective view of the actuator knob 1602 of a control mechanism. The actuator knob 1602 may comprise a control knob of the delivery system, which may comprise a release knob as disclosed herein or another form of knob of the delivery system as desired. In examples, other forms of actuators may be utilized as desired.

The actuator knob 1602 may include a first portion or inner portion 1606 and may include a second portion or outer portion 1604. The outer portion 1604 may comprise an outer body extending around the inner portion 1606. The outer portion 1604 may comprise an outer shell extending around the inner portion 1606. The outer portion 1604 may surround the inner portion 1606 or may have another configuration. The outer portion 1604 may be configured to displace relative to the inner portion 1606. The displacement may comprise a rotational displacement. The displacement may automatically limit the tension force or compression force in at least one of the shafts.

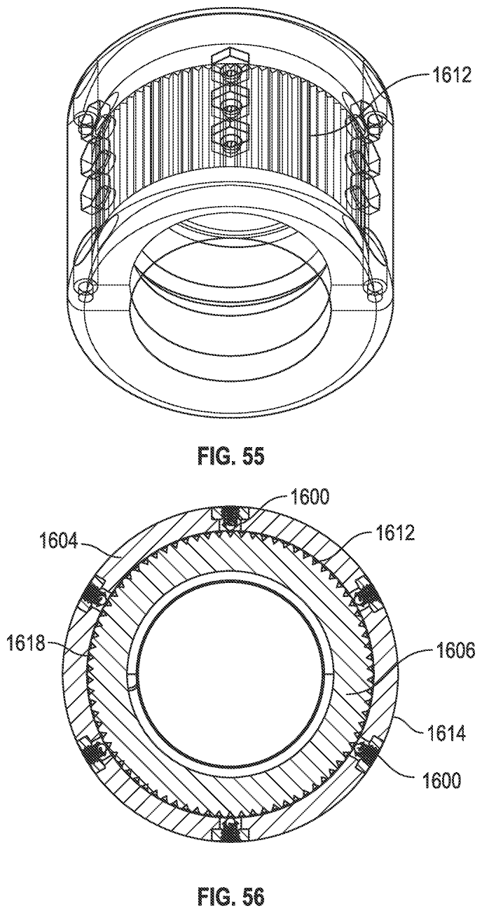

The inner portion 1606 may comprise an inner body that may be positioned within the outer portion 1604. The inner portion 1606 may comprise a sleeve configured to extend over an outer surface of a handle 14 as shown in FIG. 14, for example. The inner portion 1606 may include a central channel 1608 that the handle 14 may be positioned within. The inner portion 1606 may have an inner surface 1610 that may face towards the central channel 1608 and may have an outer surface 1612 (visible in the transparent view of FIG. 55) that may face opposite the inner surface 1610.

The outer surface 1612 may comprise an engagement surface for the displacement bodies 1600 to engage. The outer surface 1612, for example, may comprise a ridged surface for the displacement bodies 1600 to engage.

The outer portion 1604 may have an outer surface 1614. The outer surface 1614 may comprise a portion of the actuator knob 1602 or control knob for a user to grip, to apply a rotational force (i.e., a torque) upon the actuator knob 1602.

The outer portion 1604 may include an inner surface 1618 that may face opposite the outer surface 1614. The inner surface 1618 may face towards the outer surface 1612 of the inner portion 1606. The plurality of displacement bodies 1600 may protrude radially inward from the inner surface 1618 of the outer portion 1604. The displacement bodies 1600 may extend radially towards the engagement surface or outer surface 1612 of the inner portion 1606. The displacement bodies 1600 may be configured to engage the outer portion 1604 with the inner portion 1606 to retain a rotational position of the outer portion 1604 relative to the inner portion 1606.

The displacement bodies 1600 may comprise detents configured to retain a rotational position of the inner portion 1606 relative to the outer portion 1604. A detent may comprise a protrusion (e.g., a ball) configured to engage the outer surface 1612. The protrusion may be spring biased towards the outer surface 1612. For example, the displacement bodies 1600 may each comprise a spring pressing the protrusion against the engagement surface of the inner portion 1606.

FIG. 56 illustrates a cross sectional view of the outer portion 1604 positioned upon the inner portion 1606 at a view perpendicular to the view shown in FIG. 54. The engagement of the displacement bodies 1600 with the outer surface 1612 is shown. The displacement bodies 1600 may remain engaged with the outer surface 1612 due to the displacement bodies 1600 being biased towards the outer surface 1612.

The displacement bodies 1600 may release from the outer surface 1612 upon a threshold tension or compression force in a shaft (e.g., shaft 24) being produced. Longitudinal movement of the inner portion 1606 may be resisted, which may result in a resistance to rotational movement of the inner portion 1606. As such, the force of the displacement bodies 1600 against the outer surface 1612 may be overcome and the outer portion 1604 may rotate relative to the inner portion 1606. The displacement bodies 1600 may release from the outer surface 1612 to allow the surfaces 1612, 1618 to slide relative to each other. As such, the outer portion 1604 may be prevented from conveying further rotational motion to the inner portion 1606. Longitudinal movement of inner portion 1606 and an adapter (e.g., adapter 704), and correspondingly a shaft may be reduced. Upon the tension or compression force in the shaft being reduced, the displacement bodies 1600 may reengage with the outer surface 1612, thus allowing the outer portion 1604 to convey a rotational motion to the inner portion 1606.

In examples, the threshold tension force or threshold compression force that may cause the outer portion 1604 to rotate relative to the inner portion 1606 may be adjustable. The engagement force of the displacement bodies 1600 with the outer portion 1604 and inner portion 1606 may be adjusted. For example, a configuration of one or more of the displacement bodies 1600 or the outer surface 1612 may be adjusted. A size or shape of ridges of the outer surface 1612, for example, may be varied to adjust the force required to be overcome to allow for rotational motion of the portions 1604, 1606. A number or angle of the ridges may be varied as desired. In examples, the configuration of the displacement bodies 1600 may be adjusted. For example, a force of a spring of the displacement bodies 1600 may be varied to vary the amount of force upon the outer surface 1612. A number or position of the displacement bodies 1600 may be adjusted.

A configuration of the displacement bodies may be varied in examples.

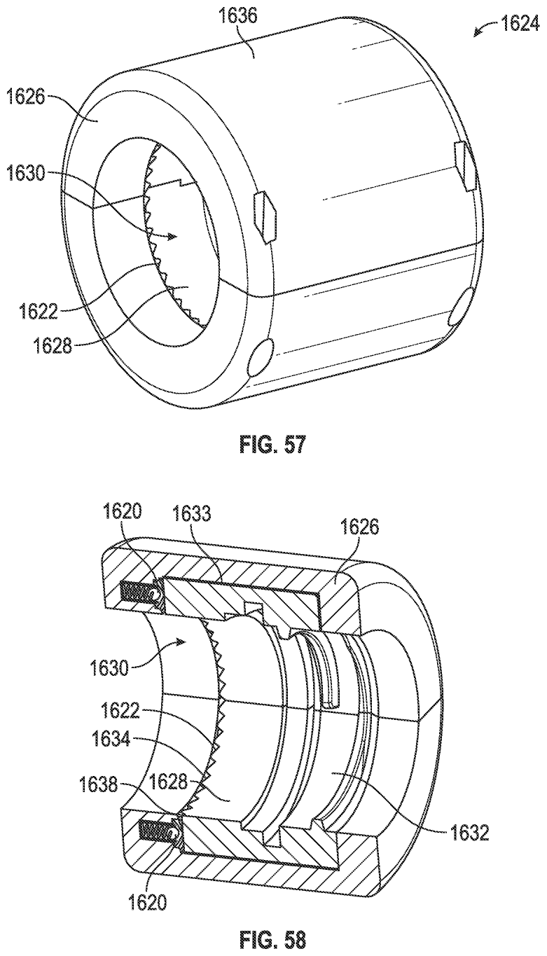

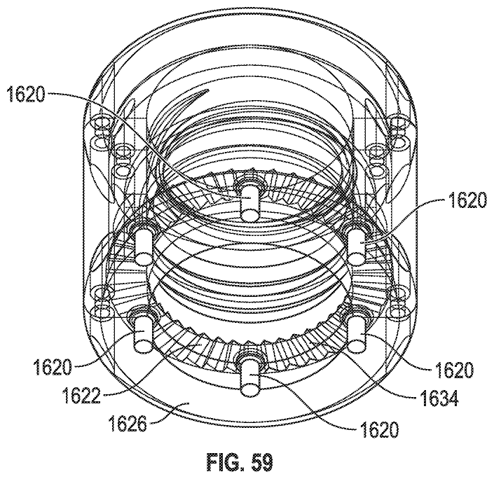

FIGS. 57-59, for example, illustrates an implementation in which a plurality of displacement bodies 1620 may be provided (shown in the transparent view of FIG. 59). The plurality of displacement bodies 1620 may be spaced from each other circumferentially. Each displacement body 1620 may extend longitudinally and may face a longitudinal direction. An engagement surface 1622 may face a longitudinal direction.

FIG. 57 illustrates a perspective view of the actuator knob 1624 of a control mechanism. The actuator knob 1624 may comprise a control knob of the delivery system, which may comprise a release knob as disclosed herein or another form of knob of the delivery system as desired. In examples, other forms of actuators may be utilized as desired.

The actuator knob 1624 may include a first portion or inner portion 1628 and may include a second portion or outer portion 1626. The outer portion 1626 may comprise an outer body extending around the inner portion 1628. The outer portion 1626 may comprise an outer shell extending around the inner portion 1628. The outer portion 1626 may surround the inner portion 1628 or may have another configuration. The outer portion 1626 may be configured to displace relative to the inner portion 1628. The displacement may comprise a rotational displacement. The displacement may automatically limit the tension force or compression force in at least one of the shafts.

The inner portion 1628 may comprise an inner body that may be positioned within the outer portion 1626. The inner portion 1628 may comprise a sleeve configured to extend over an outer surface of a handle 14 as shown in FIG. 14, for example. The inner portion 1628 may include a central channel 1630 that the handle 14 may be positioned within. The inner portion 1628 may have an inner surface 1632 that may face towards the central channel 1630 and may have an outer surface 1633 (marked in FIG. 58) that may face opposite the inner surface 1632.

The inner portion 1628 may include a longitudinally facing surface 1634 (marked in the transparent view of FIG. 59) that may comprise the engagement surface 1622 for the displacement bodies 1620 to engage. The longitudinally facing surface 1634, for example, may comprise a ridged surface for the displacement bodies 1620 to engage.

The outer portion 1626 may have an outer surface 1636. The outer surface 1636 may comprise a portion of the actuator knob 1624 or control knob for a user to grip, to apply a rotational force (i.e., a torque) upon the actuator knob 1624.

The outer portion 1626 may include a longitudinally facing surface 1638 that may face towards the longitudinally facing surface 1634 of the inner portion 1628. The displacement bodies 1620 may protrude from the longitudinally facing surface 1638 of the outer portion 1604 to engage the inner portion 1628. The displacement bodies 1620 may extend longitudinally towards the engagement surface 1622.

The engagement force of the displacement bodies 1620 with the outer portion 1626 and the inner portion 1628 may be adjusted. For example, the shape of the engagement surface 1622 can be modified to engage with displacement bodies in different manners. Such a feature may allow for modification of the threshold force as well as add an option for directionality in rotation. The spring force of the displacement bodies 1620, or the position or number of the displacement bodies 1620 may be adjusted as desired.

The displacement bodies 1620 may be configured similarly as the displacement bodies 1600 discussed in regard to FIGS. 53-56 and may operate in a similar manner. The displacement bodies 1620 may be configured to engage the outer portion 1626 with the inner portion 1628 to retain a rotational position of the outer portion 1626 relative to the inner portion 1628.

Various other configurations may be utilized as desired. The features of the examples of FIGS. 43-59 may be utilized in combination across examples. For example, features utilized for an example may be utilized with any other example. Features may be modified or combined as desired.

The examples of FIGS. 43-59 may be utilized with a manifold shaft or manifold subassembly 24, or may be utilized with any other shaft or assembly or subassembly of a delivery system. For example, the examples of FIGS. 43-59 may be implemented with an outer sheath or outer sheath subassembly 20, or any other shaft or assembly or subassembly disclosed herein. The rail subassembly 21, mid-shaft subassembly 22, release subassembly 23, or nose cone assembly may utilize the examples of FIGS. 43-59. Any feature of a control mechanism, such as a knob disclosed herein may be utilized in combination or utilized in alternative with the examples of FIGS. 43-59. Any feature of an adapter disclosed herein may be utilized in combination or utilized in alternative with the examples of FIGS. 43-59.

From the foregoing description, it will be appreciated that apparatuses, devices, systems, methods are disclosed. While several components, techniques and aspects have been described with a certain degree of particularity, it is manifest that many changes can be made in the specific designs, constructions and methodology herein above described without departing from the spirit and scope of this disclosure.

For purposes of this description, certain aspects, advantages, and novel features of the examples of this disclosure are described herein. The disclosed methods, apparatuses, and systems should not be construed as limiting in any way. Instead, the present disclosure is directed toward all novel and nonobvious features and aspects of the various disclosed examples, along and in various combinations and sub-combinations with one another. The methods, apparatuses, and systems are not limited to any specific aspect or feature or combination thereof, nor do the disclosed examples require that any one or more specific advantages be present or problems be solved. Features, elements, or combinations of one example can be combined into other examples herein.

Example 1: A delivery system for an implant, the delivery system comprising: an elongate catheter including an implant retention area for retaining the implant, the elongate catheter including one or more shafts; a control mechanism for moving the one or more shafts; and a force reduction mechanism configured to automatically reduce a tension force or a compression force in at least one of the one or more shafts.

Example 2: The delivery system of any example herein, in particular Example 1, wherein the force reduction mechanism is configured to allow the at least one of the one or more shafts to move proximally or distally relative to the control mechanism to automatically reduce the tension force or the compression force in the at least one of the one or more shafts.

Example 3: The delivery system of any example herein, in particular Example 1 or Example 2, wherein the force reduction mechanism includes a displacement body configured to allow the at least one of the one or more shafts to move proximally or distally relative to the control mechanism to automatically reduce the tension force or the compression force in the at least one of the one or more shafts.

Example 4: The delivery system of any example herein, in particular Example 3, wherein the displacement body comprises a spring.

Example 5: The delivery system of any example herein, in particular Example 4, wherein the control mechanism includes an actuator and the force reduction mechanism includes an adapter at a proximal portion of the at least one of the one or more shafts configured to engage the actuator, and the spring is positioned on the adapter.

Example 6: The delivery system of any example herein, in particular Example 4 or Example 5, wherein the control mechanism includes an actuator and the spring is positioned on the actuator.

Example 7: The delivery system of any example herein, in particular Examples 1-6, wherein the control mechanism includes an actuator and the force reduction mechanism includes an adapter at a proximal portion of the at least one of the one or more shafts configured to engage the actuator, and the one or more shafts is configured to displace relative to one or more of the actuator or the adapter to automatically reduce the tension force or the compression force in the at least one of the one or more shafts.

Example 8: The delivery system of any example herein, in particular Examples 1-7, wherein the control mechanism includes an actuator and the actuator includes a first portion and a second portion that is configured to displace relative to the first portion to automatically reduce the tension force or the compression force in the at least one of the one or more shafts.

Example 9: The delivery system of any example herein, in particular Examples 1-8, wherein the control mechanism includes an actuator and the force reduction mechanism includes an adapter at a proximal portion of the at least one of the one or more shafts engaged with the actuator and configured to disengage from the actuator to automatically reduce the tension force or the compression force in the at least one of the one or more shafts.

Example 10: The delivery system of any example herein, in particular Example 9, wherein the force reduction mechanism includes a cam configured to disengage the adapter from the actuator.

Example 11: The delivery system of any example herein, in particular Example 9 or Example 10, wherein the force reduction mechanism includes a spring pressing the adapter towards the actuator and configured to be moved to disengage the adapter from the actuator.

Example 12: The delivery system of any example herein, in particular Examples 1-11, wherein the force reduction mechanism is configured to be user actuated.

Example 13: The delivery system of any example herein, in particular Examples 1-12, wherein the force reduction mechanism is configured to automatically reduce the tension force or the compression force in the at least one of the one or more shafts upon a respective threshold tension force or threshold compression force being met.

Example 14: The delivery system of any example herein, in particular Examples 1-13, further comprising an indicator indicating the tension force or the compression force in the at least one of the one or more shafts.

Example 15: The delivery system of any example herein, in particular Examples 1-14, wherein the force reduction mechanism includes an electric drive configured to automatically reduce the tension force or the compression force in the at least one of the one or more shafts by moving the at least one of the one or more shafts.

Example 16: The delivery system of any example herein, in particular Example 15, wherein the electric drive is configured to automatically move the at least one of the one or more shafts distally to reduce the tension force or automatically move the at least one of the one or more shafts proximally to reduce the compression force.

Example 17: The delivery system of any example herein, in particular Examples 1-16, wherein the force reduction mechanism is configured to automatically reduce the tension force or the compression force in the at least one of the one or more shafts comprising an outer sheath covering the implant retention area.

Example 18: The delivery system of any example herein, in particular Examples 1-17, wherein the force reduction mechanism is configured to automatically reduce the tension force or the compression force in the at least one of the one or more shafts comprising a manifold shaft coupled to one or more sutures for coupling to the implant.

Example 19: The delivery system of any example herein, in particular Examples 1-18, wherein the elongate catheter includes a handle, and the control mechanism includes an actuator knob positioned on the handle, and the force reduction mechanism is configured to automatically allow the actuator knob to rotate relative to the handle to reduce the tension force or the compression force in the at least one of the one or more shafts.

Example 20: The delivery system of any example herein, in particular Examples 1-19, wherein the force reduction mechanism is configured to automatically allow the at least one of the one or more shafts to move distally to reduce a tension force produced by the at least one of the one or shafts being moved proximally by the control mechanism.

Example 21: The delivery system of any example herein, in particular Examples 1-20, wherein the force reduction mechanism is configured to automatically allow the at least one of the one or more shafts to move proximally to reduce a compression force produced by the at least one of the one or shafts being moved distally by the control mechanism.

Example 22: The delivery system of any example herein, in particular Examples 1-21, wherein the control mechanism is for moving a first shaft of the one or more shafts, and the force reduction mechanism is configured to automatically reduce the tension force or the compression force in the first shaft.

Example 23: The delivery system of any example herein, in particular Examples 1-22, wherein the elongate catheter includes a plurality of the shafts, and the control mechanism is for moving a first shaft of the plurality of the shafts, and the force reduction mechanism is configured to automatically reduce the tension force or the compression force in a second shaft of the plurality of the shafts.

Example 24: The delivery system of any example herein, in particular Example 23, wherein the control mechanism is for moving each of the plurality of the shafts.

Example 25: The delivery system of any example herein, in particular Examples 1-24, wherein the elongate catheter is configured to deliver the implant comprising a prosthetic heart valve to a heart valve of a patient's body.

Example 26: A delivery system for an implant, the delivery system comprising: an elongate catheter including an implant retention area for retaining the implant, the elongate catheter including one or more shafts; a control mechanism for moving the one or more shafts; and an indicator indicating a tension force or a compression force in at least one of the one or more shafts.

Example 27: The delivery system of any example herein, in particular Example 26, wherein the indicator comprises a displacement of at least two bodies from each other.

Example 28: The delivery system of any example herein, in particular Example 27, wherein the indicator includes a spring coupling the at least two bodies to each other.

Example 29: The delivery system of any example herein, in particular Example 27 or Example 28, wherein the control mechanism includes an actuator, and the at least two bodies comprise portions of the actuator.

Example 30: The delivery system of any example herein, in particular Examples 27-29, wherein the control mechanism includes an actuator and an adapter is positioned at a proximal portion of the at least one of the one or more shafts for engaging the actuator, and the at least two bodies comprise the actuator and the adapter.

Example 31: The delivery system of any example herein, in particular Example 29 or Example 30, wherein the actuator comprises an actuator knob.

Example 32: The delivery system of any example herein, in particular Examples 26-31, wherein the indicator comprises one or more of a visual indicator, an auditory indicator, or a haptic indicator.

Example 33: The delivery system of any example herein, in particular Examples 26-32, wherein the indicator comprises an electric indicator.

Example 34: The delivery system of any example herein, in particular Example 33, wherein the electric indicator comprises an electric display.

Example 35: The delivery system of any example herein, in particular Examples 26-34, wherein the elongate catheter is configured to deliver the implant comprising a prosthetic heart valve to a heart valve of a patient's body.

Example 36: A method comprising: utilizing a delivery system to deploy an implant within a patient's body, the delivery system including: an elongate catheter including an implant retention area retaining the implant, the elongate catheter including one or more shafts, a control mechanism for moving the one or more shafts, and a force reduction mechanism configured to automatically reduce a tension force or a compression force in at least one of the one or more shafts.

Example 37: The method of any example herein, in particular Example 36, wherein the force reduction mechanism is configured to allow the at least one of the one or more shafts to move proximally or distally relative to the control mechanism to automatically reduce the tension force or the compression force in the at least one of the one or more shafts.

Example 38: The method of any example herein, in particular Example 36 or Example 37, wherein the force reduction mechanism includes a displacement body configured to allow the at least one of the one or more shafts to move proximally or distally relative to the control mechanism to automatically reduce the tension force or the compression force in the at least one of the one or more shafts.

Example 39: The method of any example herein, in particular Example 38, wherein the displacement body comprises a spring.

Example 40: The method of any example herein, in particular Example 39, wherein the control mechanism includes an actuator and the force reduction mechanism includes an adapter at a proximal portion of the at least one of the one or more shafts configured to engage the actuator, and the spring is positioned on the adapter.

Example 41: The method of any example herein, in particular Example 39 or Example 40, wherein the control mechanism includes an actuator and the spring is positioned on the actuator.

Example 42: The method of any example herein, in particular Examples 36-41, wherein the control mechanism includes an actuator and the force reduction mechanism includes an adapter at a proximal portion of the at least one of the one or more shafts configured to engage the actuator, and the one or more shafts are configured to displace relative to one or more of the actuator or the adapter to automatically reduce the tension force or the compression force in the at least one of the one or more shafts.

Example 43: The method of any example herein, in particular Examples 36-42, wherein the control mechanism includes an actuator and the actuator includes a first portion and a second portion that is configured to displace relative to the first portion to automatically reduce the tension force or the compression force in the at least one of the one or more shafts.

Example 44: The method of any example herein, in particular Examples 36-43, wherein the control mechanism includes an actuator and the force reduction mechanism includes an adapter at a proximal portion of the at least one of the one or more shafts engaged with the actuator and configured to disengage from the actuator to automatically reduce the tension force or the compression force in the at least one of the one or more shafts.

Example 45: The method of any example herein, in particular Example 44, wherein the force reduction mechanism includes a cam configured to disengage the adapter from the actuator.

Example 46: The method of any example herein, in particular Example 44 or Example 45, wherein the force reduction mechanism includes a spring pressing the adapter towards the actuator and configured to be compressed to disengage the adapter from the actuator.

Example 47: The method of any example herein, in particular Examples 36-46, wherein the force reduction mechanism is configured to be user actuated.

Example 48: The method of any example herein, in particular Examples 36-47, wherein the force reduction mechanism is configured to automatically reduce the tension force or the compression force in the at least one of the one or more shafts upon a respective threshold tension force or threshold compression force being met.

Example 49: The method of any example herein, in particular Examples 36-48, wherein an indicator is configured to indicate the tension force or the compression force in the at least one of the one or more shafts.

Example 50: The method of any example herein, in particular Examples 36-49, wherein the force reduction mechanism includes an electric drive configured to automatically reduce the tension force or the compression force in the at least one of the one or more shafts by moving the at least one of the one or more shafts.

Example 51: The method of any example herein, in particular Example 50, wherein the electric drive is configured to automatically move the at least one of the one or more shafts distally to reduce the tension force or automatically move the at least one of the one or more shafts proximally to reduce the compression force.

Example 52: The method of any example herein, in particular Examples 36-51, wherein the force reduction mechanism is configured to automatically reduce the tension force or the compression force in the at least one of the one or more shafts comprising an outer sheath covering the implant retention area.

Example 53: The method of any example herein, in particular Examples 36-52, wherein the force reduction mechanism is configured to automatically reduce the tension force or the compression force in the at least one of the one or more shafts comprising a manifold shaft coupled to one or more sutures for coupling to the implant.

Example 54: The method of any example herein, in particular Examples 36-53, wherein the elongate catheter includes a handle, and the control mechanism includes an actuator knob positioned on the handle, and the force reduction mechanism is configured to automatically allow the actuator knob to rotate relative to the handle to reduce the tension force or the compression force in the at least one of the one or more shafts.

Example 55: The method of any example herein, in particular Examples 36-54, wherein the force reduction mechanism is configured to automatically allow the at least one of the one or more shafts to move distally to reduce a tension force produced by the at least one of the one or shafts being moved proximally by the control mechanism.

Example 56: The method of any example herein, in particular Examples 36-55, wherein the force reduction mechanism is configured to automatically allow the at least one of the one or more shafts to move proximally to reduce a compression force produced by the at least one of the one or shafts being moved distally by the control mechanism.

Example 57: The method of any example herein, in particular Examples 36-56, wherein the control mechanism is for moving a first shaft of the one or more shafts, and the force reduction mechanism is configured to automatically reduce the tension force or the compression force in the first shaft.

Example 58: The method of any example herein, in particular Examples 36-57, wherein the elongate catheter includes a plurality of the shafts, and the control mechanism is for moving a first shaft of the plurality of the shafts, and the force reduction mechanism is configured to automatically reduce the tension force or the compression force in a second shaft of the plurality of the shafts.

Example 59: The method of any example herein, in particular Example 58, wherein the control mechanism is for moving each of the plurality of the shafts.

Example 60: The method of any example herein, in particular Examples 36-59, wherein the elongate catheter is configured to deliver the implant comprising a prosthetic heart valve to a heart valve of the patient's body.

Example 61: A method comprising: utilizing a delivery system to deploy an implant within a patient's body, the delivery system including: an elongate catheter including an implant retention area for retaining the implant, the elongate catheter including one or more shafts, a control mechanism for moving the one or more shafts, and an indicator indicating a tension force or a compression force in at least one of the one or more shafts.

Example 62: The method of any example herein, in particular Example 61, wherein the indicator comprises a displacement of at least two bodies from each other.

Example 63: The method of any example herein, in particular Example 62, wherein the indicator includes a spring coupling the at least two bodies to each other.

Example 64: The method of any example herein, in particular Example 62 or Example 63, wherein the control mechanism includes an actuator, and the at least two bodies comprise portions of the actuator.

Example 65: The method of any example herein, in particular Examples 62-64, wherein the control mechanism includes an actuator and an adapter is positioned at a proximal portion of the at least one of the one or more shafts for engaging the actuator, and the at least two bodies comprise the actuator and the adapter.

Example 66: The method of any example herein, in particular Example 64 or Example 65, wherein the actuator comprises an actuator knob.

Example 67: The method of any example herein, in particular Examples 61-66, wherein the indicator comprises one or more of a visual indicator, an auditory indicator, or a haptic indicator.

Example 68: The method of any example herein, in particular Examples 61-67, wherein the indicator comprises an electric indicator.

Example 69: The method of any example herein, in particular Example 68, wherein the electric indicator comprises an electric display.

Example 70: The method of any example herein, in particular Examples 61-69, wherein the elongate catheter is configured to deliver the implant comprising a prosthetic heart valve to a heart valve of the patient's body.

Example 71: A delivery system for an implant, the delivery system comprising: an elongate catheter including an implant retention area for retaining the implant, the elongate catheter including one or more shafts; and a control mechanism for moving the one or more shafts, the control mechanism including an actuator knob having a first portion and a second portion that is configured to rotate relative to the first portion to automatically limit a tension force or a compression force in at least one of the one or more shafts transmitted by the actuator knob to the at least one of the one or more shafts.

Example 72: The delivery system of any example herein, in particular Example 71, wherein the first portion comprises an inner body and the second portion comprises an outer body extending around the inner body.

Example 73: The delivery system of any example herein, in particular Example 72, wherein the outer body comprises an outer shell.

Example 74: The delivery system of any example herein, in particular Example 72 or Example 73, wherein the inner body is configured to engage an adapter coupled to a proximal end portion of the at least one of the one or more shafts.

Example 75: The delivery system of any example herein, in particular Example 74, wherein the inner body is configured to rotate about the adapter and convey rotational motion of the actuator knob to a longitudinal motion of the adapter.

Example 76: The delivery system of any example herein, in particular Examples 71-75, wherein the first portion or the second portion includes a bearing surface configured to allow the first portion to rotate relative to the second portion.

Example 77: The delivery system of any example herein, in particular Example 76, wherein the bearing surface has a varied diameter.

Example 78: The delivery system of any example herein, in particular Examples 71-77, further comprising a displacement body configured to engage the second portion with the first portion to retain a rotational position of the second portion relative to the first portion.

Example 79: The delivery system of any example herein, in particular Example 78, wherein the displacement body is biased towards an engagement surface of the first portion or the second portion.

Example 80: The delivery system of any example herein, in particular Example 79, wherein the displacement body is spring biased.

Example 81: The delivery system of any example herein, in particular Example 79 or Example 80, wherein the displacement body extends radially towards the engagement surface.

Example 82: The delivery system of any example herein, in particular Examples 79-81, wherein the displacement body extends longitudinally towards the engagement surface.

Example 83: The delivery system of any example herein, in particular Examples 71-82, wherein the second portion is configured to rotate relative to the first portion upon a threshold tension force or a threshold compression force being met.

Example 84: The delivery system of any example herein, in particular Example 83, wherein the threshold tension force or the threshold compression force is adjustable.

Example 85: The delivery system of any example herein, in particular Example 84, wherein the first portion comprises an inner body and the second portion comprises an outer shell extending around the inner body, and the threshold tension force or the threshold compression force is adjustable by adjusting a compression of the outer shell upon the inner body.

Example 86: The delivery system of any example herein, in particular Example 84 or Example 85, further comprising a displacement body configured to engage the second portion with the first portion to retain a rotational position of the second portion relative to the first portion, and the threshold tension force or the threshold compression force is adjustable by adjusting an engagement force of the displacement body with the first portion and the second portion.

Example 87: The delivery system of any example herein, in particular Examples 71-86, wherein the second portion is configured to rotate relative to the first portion to prevent a user from exceeding a respective threshold tension force or threshold compression force in the at least one of the one or more shafts.

Example 88: The delivery system of any example herein, in particular Examples 71-87, wherein the at least one of the one or more shafts comprises a manifold shaft coupled to one or more sutures for coupling to the implant.

Example 89: The delivery system of any example herein, in particular Examples 71-88, wherein the at least one of the one or more shafts comprises an outer sheath covering the implant retention area.

Example 90: The delivery system of any example herein, in particular Examples 71-89, wherein the elongate catheter is configured to deliver the implant comprising a prosthetic heart valve to a heart valve of a patient's body.

Example 91: A method comprising: utilizing a delivery system to deploy an implant within a patient's body, the delivery system including: an elongate catheter including an implant retention area for retaining the implant, the elongate catheter including one or more shafts, and a control mechanism for moving the one or more shafts, the control mechanism including an actuator knob having a first portion and a second portion that is configured to rotate relative to the first portion to automatically limit a tension force or a compression force in at least one of the one or more shafts transmitted by the actuator knob to the at least one of the one or more shafts.

Example 92: The method of any example herein, in particular Example 91, wherein the first portion comprises an inner body and the second portion comprises an outer body extending around the inner body.

Example 93: The method of any example herein, in particular Example 91 or Example 92, wherein the first portion or the second portion includes a bearing surface configured to allow the first portion to rotate relative to the second portion.

Example 94: The method of any example herein, in particular Examples 91-93, wherein a displacement body is configured to engage the second portion with the first portion to retain a rotational position of the second portion relative to the first portion.

Example 95: The method of any example herein, in particular Example 94, wherein the displacement body extends radially towards an engagement surface.

Example 96: The method of any example herein, in particular Example 94 or Example 95, wherein the displacement body extends longitudinally towards an engagement surface.

Example 97: The method of any example herein, in particular Examples 91-96, wherein the second portion is configured to rotate relative to the first portion upon a threshold tension force or a threshold compression force being met.

Example 98: The method of any example herein, in particular Example 97, wherein the threshold tension force or the threshold compression force is adjustable.

Example 99: The method of any example herein, in particular Examples 91-98, wherein the second portion is configured to rotate relative to the first portion to prevent a user from exceeding a respective threshold tension force or threshold compression force in the at least one of the one or more shafts.

Example 100: The method of any example herein, in particular Examples 91-99, wherein the elongate catheter is configured to deliver the implant comprising a prosthetic heart valve to a heart valve of the patient's body.

Example 101: A delivery system for an implant, the delivery system comprising: an elongate catheter including an implant retention area for retaining the implant, the elongate catheter including at least one shaft; an actuator knob on a handle for advancing or retracting the at least one shaft, the actuator knob engaged with the at least one shaft; a force reduction mechanism for allowing the at least one shaft to disengage from the actuator knob for reducing a tension force in the at least one shaft; wherein the force reduction mechanism causes the at least one shaft to disengage from the actuator knob upon reaching a threshold tension force for allowing the at least one shaft to slide longitudinally relative to the actuator knob; and wherein the force reduction mechanism reduces damage to the at least one shaft.

Example 102: The delivery system of any example herein, in particular Example 101, wherein the force reduction mechanism includes an adapter at a proximal portion of the at least one shaft for engaging the actuator knob.

Example 103: The delivery system of any example herein, in particular Example 102, wherein the force reduction mechanism includes one or more protrusions extending radially outward from the adapter for engaging the actuator knob.

Example 104: The delivery system of any example herein, in particular Example 103, wherein the force reduction mechanism includes one or more springs biasing the one or more protrusions radially outward towards the actuator knob.

Example 105: The delivery system of any example herein, in particular Example 103 or Example 104, wherein the actuator knob includes an interior surface for engaging the one or more protrusions.

Example 106: The delivery system of any example herein, in particular Example 105, wherein the one or more protrusions displace inward from the interior surface to disengage the at least one shaft from the actuator knob.

Example 107: The delivery system of any example herein, in particular Examples 101-106, further comprising an indicator for indicating the tension force in the at least one shaft.

Example 108: The delivery system of any example herein, in particular Examples 101-107, wherein the force reduction mechanism automatically reduces the tension force in the at least one shaft comprising an outer sheath covering the implant retention area.

Example 109: The delivery system of any example herein, in particular Examples 101-108, wherein the force reduction mechanism automatically reduces the tension force in the at least one shaft comprising a manifold shaft coupled to one or more sutures for coupling to the implant.

Example 110: The delivery system of any example herein, in particular Examples 101-109, wherein the elongate catheter is for delivering the implant comprising a prosthetic heart valve to a heart valve of a patient's body.

Any of the features of any of the examples, including but not limited to any of the first through 110 examples referred to above, is applicable to all other aspects and examples identified herein, including but not limited to any examples of any of the first through 110 examples referred to above. Moreover, any of the features of an example of the various examples, including but not limited to any examples of any of the first through 110 examples referred to above, is independently combinable, partly or wholly with other examples described herein in any way, e.g., one, two, or three or more examples may be combinable in whole or in part. Further, any of the features of the various examples, including but not limited to any examples of any of the first through 110 examples referred to above, may be made optional to other examples. Any example of a method can be performed by a system or apparatus of another example, and any aspect or example of a system or apparatus can be configured to perform a method of another aspect or example, including but not limited to any examples of any of the first through 110 examples referred to above.

Certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as any subcombination or variation of any subcombination.

Moreover, while methods may be depicted in the drawings or described in the specification in a particular order, such methods need not be performed in the particular order shown or in sequential order, and that all methods need not be performed, to achieve desirable results. Other methods that are not depicted or described can be incorporated in the example methods and processes. For example, one or more additional methods can be performed before, after, simultaneously, or between any of the described methods. Further, the methods may be rearranged or reordered in other implementations. Also, the separation of various system components in the implementations described above should not be understood as requiring such separation in all implementations, and it should be understood that the described components and systems can generally be integrated together in a single product or packaged into multiple products. Additionally, other implementations are within the scope of this disclosure.

Conditional language, such as “can,” “could,” “might,” or “may,” unless specifically stated otherwise, or otherwise understood within the context as used, is generally intended to convey that certain examples include or do not include, certain features, elements, and/or steps. Thus, such conditional language is not generally intended to imply that features, elements, and/or steps are in any way required for one or more examples.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain examples require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than or equal to 10% of, within less than or equal to 5% of, within less than or equal to 1% of, within less than or equal to 0.1% of, and within less than or equal to 0.01% of the stated amount. If the stated amount is 0 (e.g., none, having no), the above recited ranges can be specific ranges, and not within a particular % of the value. For example, within less than or equal to 10 wt./vol. % of, within less than or equal to 5 wt./vol. % of, within less than or equal to 1 wt./vol. % of, within less than or equal to 0.1 wt./vol. % of, and within less than or equal to 0.01 wt./vol. % of the stated amount.

Some examples have been described in connection with the accompanying drawings. The figures are drawn to scale, but such scale should not be limiting, since dimensions and proportions other than what are shown are contemplated and are within the scope of the disclosed inventions. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various examples can be used in all other examples set forth herein. Additionally, it will be recognized that any methods described herein may be practiced using any device suitable for performing the recited steps.

While a number of examples and variations thereof have been described in detail, other modifications and methods of using the same will be apparent to those of skill in the art. Accordingly, it should be understood that various applications, modifications, materials, and substitutions can be made of equivalents without departing from the unique and inventive disclosure herein or the scope of the claims.

What is claimed is:

1. A delivery system for an implant, the delivery system comprising:
a handle;
an elongate catheter coupled to the handle, the elongate catheter including one or more shafts and including an implant retention area for retaining the implant;
a control mechanism on the handle for longitudinally sliding the one or more shafts; and
a force reduction mechanism that is selectively actuatable for reducing a force in at least one of the one or more shafts, wherein the force reduction mechanism is adapted to reduce the force upon a longitudinal threshold force being met in the at least one of the one or more shafts.

2. The delivery system of claim 1, wherein the control mechanism includes an actuator for controlling the longitudinal sliding of the one or more shafts.

3. The delivery system of claim 2, wherein the force reduction mechanism includes an adapter engaged with the at least one of the one or more shafts, the adapter being selectively engaged with the actuator, wherein the adapter is capable of disengaging from the actuator to thereby allow relative movement between the actuator and the at least one of the one or more shafts.

4. The delivery system of claim 3, wherein the actuator comprises a knob.

5. The delivery system of claim 3, further comprising a control device for disengaging the adapter from the actuator.

6. The delivery system of claim 1, further comprising an indicator for indicating a longitudinal force in the at least one of the one or more shafts.

7. The delivery system of claim 1, wherein the at least one of the one or more shafts is an outer sheath for covering the implant retention area.

8. The delivery system of claim 1, wherein the at least one of the one or more shafts is a manifold shaft coupled to one or more sutures for coupling to the implant.

9. The delivery system of claim 1, further comprising:
an actuator knob on the handle engaged with and for advancing or retracting at least one of the one or more shafts, the actuator knob including a proximal portion and a distal portion; and
a releasable coupler configured to engage an elastomeric body to releasably couple the proximal portion of the actuator knob to the distal portion of the actuator knob, the releasable coupler extending between the proximal portion and the distal portion, and
wherein the releasable coupler is adapted to disengage from the elastomeric body to allow the proximal portion to disengage from the distal portion upon a longitudinal threshold force being met in the at least one of the one or more shafts engaged with the actuator knob.

10. The delivery system of claim 1, wherein the elongate catheter is for delivering the implant comprising a prosthetic heart valve to a heart valve of a patient's body.

11. A delivery system for an implant, the delivery system comprising:
an elongate catheter including an implant retention area for retaining the implant, the elongate catheter including one or more shafts; and
a control mechanism for moving the one or more shafts, the control mechanism including an actuator knob having an inner body and an outer body extending around the inner body, the outer body adapted to slip relative to the inner body to limit a tension force or a compression force in at least one of the one or more shafts transmitted by the actuator knob to the at least one of the one or more shafts.

12. The delivery system of claim 11, wherein the outer body comprises an outer shell.

13. The delivery system of claim 11, wherein the inner body is for engaging an adapter coupled to a proximal end portion of the at least one of the one or more shafts.

14. The delivery system of claim 13, wherein the inner body is for rotating about the adapter and conveying rotational motion of the actuator knob to a longitudinal motion of the adapter.

15. The delivery system of claim 11, wherein the inner body or the outer body includes a bearing surface for allowing the inner body to rotate relative to the outer body.

16. The delivery system of claim 11, further comprising a displacement body for engaging the outer body with the inner body to retain a rotational position of the outer body relative to the inner body, and wherein the displacement body is biased towards an engagement surface of the outer body or the inner body.

17. The delivery system of claim 16, wherein the displacement body extends radially towards the engagement surface.

18. A delivery system for implanting a medical device in a body, the delivery system comprising:
a handle;
an elongate catheter coupled to the handle, the elongate catheter including an inner shaft and an outer shaft, wherein the inner shaft is coupled to the medical device and wherein the outer shaft has an implant retention area along a distal end portion for retaining the medical device in a compressed configuration;
an actuator on the handle for longitudinally sliding the outer shaft relative to the handle via a threaded engagement; and
a force reduction mechanism that is selectively actuatable for reducing axial tension or compression in the outer shaft by disengaging the threaded engagement between the actuator and the outer shaft.

19. The delivery system of claim 18, wherein the force reduction mechanism comprises an adapter coupled to a proximal end portion of the outer shaft and having a threaded surface sized for engagement with the actuator, wherein the adapter is operable to disengage from the actuator.

20. The delivery system of claim 19, wherein the threaded surface of the adapter is spring biased for engaging the actuator during movement of the outer shaft, and the threaded surface of the adapter is adapted to disengage from the actuator upon a control device adjusting a position of the adapter.
